(19) United States
(12) Patent Application Publication
(10) Pub. No.: US 2014/0171759 A1
(43) Pub. Date: Jun. 19, 2014

(54) NONINVASIVE DETERMINATION OF INTRAVASCULAR AND EXCTRAVASCULAR HYDRATION USING NEAR INFRARED SPECTROSCOPY

(71) Applicants: Craig William White, Grosse Pointe, MI (US); Trent Daniel Ridder, Clovis, NM (US); Derek F. Treese, Riverside, CA (US); Glenn Leo Price, Riverside, CA (US); Benjamin Ver Steeg, Redlands, CA (US); Bentley Laaksonen, Albuquerque, NM (US)

(72) Inventors: Craig William White, Grosse Pointe, MI (US); Trent Daniel Ridder, Clovis, NM (US); Derek F. Treese, Riverside, CA (US); Glenn Leo Price, Riverside, CA (US); Benjamin Ver Steeg, Redlands, CA (US); Bentley Laaksonen, Albuquerque, NM (US)

(21) Appl. No.: 14/164,782

(22) Filed: Jan. 27, 2014

Related U.S. Application Data
(63) Continuation-in-part of application No. 13/767,313, filed on Feb. 14, 2013.
(60) Provisional application No. 61/899,241, filed on Nov. 3, 2013.
(60) Provisional application No. 61/599,312, filed on Feb. 15, 2012.

(51) Int. Cl. A61B 5/00

(52) U.S. Cl. CPC A61B 5/443; A61B 5/0075
USPC 600/306

(57) ABSTRACT

An apparatus and method for non-invasive determination of hydration, hydration state, total body water, or water concentration by quantitative spectroscopy. The system includes subsystems optimized to contend with the complexities of the tissue spectroscopy, high signal-to-noise ratio and photometric accuracy requirements, tissue sampling errors, calibration maintenance, and calibration transfer. The subsystems include an illumination subsystem, a tissue sampling subsystem, a spectrometer subsystem, a data acquisition subsystem, a computing subsystem, and a calibration subsystem.

### CROSS-REFERENCE TO RELATED APPLICATIONS

This application claims the benefit of U.S. Provisional Applications No. 61/599,312, filed Feb. 15, 2012, and No. 61/899,241, filed Nov. 3, 2013; and is a continuation in part of U.S. 2013/0210058, Ser. No. 13/767,313, filed Feb. 14, 2013, which claimed benefit to U.S. Provisional Application No. 61/599,312, filed Feb. 15, 2012. The entire disclosures of the above applications are incorporated herein by reference.

### FIELD

The present disclosure relates to the use of quantitative spectroscopy for measuring the presence or concentration of water and/or hydration in biological tissue and/or humans utilizing non-invasive techniques in combination with multivariate analysis.

### BACKGROUND

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Proper hydration is an important aspect of the health of individuals. Poor hydration can manifest in a variety of ways with differing degrees of severity. Some symptoms of body water loss are:

At 1% body water loss: there are few outward symptoms, however, there is a marked reduction in VO2 max.

At 2% body water loss: thirst, loss of endurance capacity and appetite.

At 3% body water loss: dry mouth; performance impaired.

At 4% body water loss: increased effort for exercise, impatience, apathy, vague discomfort, and loss of appetite.

At 5% body water loss: difficulty concentrating, increased pulse and breathing, slowing of pace.

At 6-7% body water loss: further impairment of temperature regulation, higher pulse and breathing, flushed skin, sleepiness, tingling, stumbling, headache.

At 8-9% body water loss: dizziness, labored breathing, mental confusion, and further weakness.

At 10% body water loss: muscle spasms, loss of balance, swelling of tongue.

At 11% body water loss, heat exhaustion, delirium, stroke, and difficulty swallowing; death can occur.

In the elderly or ill, these problems can be exacerbated by the tendency of elderly bodies to contain a higher proportion of fat cells, which naturally hold less water, placing these people in a perpetually mild dehydrated state. Athletes and soldiers are also affected by their hydration level and require monitoring to avoid serious complications. Hydration monitoring can also be used as a diagnostic for training that can indicate the state and quality of a workout or training regimen. Hydration state can also be an indicator of shock or trauma. Thus, when combined with a means for communicating hydration results, a hydration sensor is useful to the military for determining the condition of troops in the field. As a result, a hydration sensor would be a useful fitness for duty metric for the military. Further, given the changing medical environment, it is desirable to diagnose hydration issues early and prevent unnecessary complications and trips to an emergency room or medical facility by allowing a person to monitor their hydration levels and immediately intake fluids based on the monitoring results.

There are several existing methods for determining total body water (TBW) and hydration known in the art. Each suffers from one or more deficiencies that limit their utility such as the need for laboratory equipment, trained measurement personnel, invasive specimen acquisition, or susceptibility to inaccurate/indirect results. Isotope analysis can be used to measure TBW and hydration by directly measuring doubly labeled water (DLW) or other dilution techniques. Isotope analysis is generally well regarded in terms of accuracy and precision. Unfortunately, the isotope methods require very expensive, laboratory-based equipment. For cost and practicality, alternative methods are used to measure body water in practice.

Bioelectrical impedance analysis (BIA) is used for body composition analysis. In single-frequency mode it can provide a measure of TBW, while differentiation into both intracellular and extracellular compartments is possible if a multi-frequency device is used. In BIA, current is applied at different frequencies and the higher conductivity of water compared to other compartments is used to assess volume. However, the scientific community claims that the differentiation into intracellular and extracellular water is not proven since it is based on theory and not proven biophysical principles. Furthermore, BIA exhibits a measurement precision that is unsuitable for water losses of less than 1 L.

Two measurements based on plasma are commonly used for hydration assessment, both of which reflect water content in extracellular fluid. Under controlled settings a reduction in plasma volume, as estimated from hemoglobin and hematocrit changes, has been related to hydration. Plasma osmolality is more commonly used, and is considered to be a gold standard measurement. However, plasma osmolality is influenced by several factors, which means that this measurement must also be carried out under controlled conditions where body fluids are stable and equilibrated. As a result, plasma osmolality is not suitable for hydration measurements in dynamic environments.

Measurements from urine have been used as hydration markers including color, osmolality, and specific gravity. As with most existing hydration methods, the applicability of urine measurements is limited during periods of rapid body fluid turnover. Urine markers can lag behind plasma osmolality and weight loss, for example during intense exercise. As a result, urine hydration measurements are not suitable for dynamic environments.

Weight loss is commonly used as a marker of hydration status during short-term experiments, since it is a noninvasive and straightforward measurement. Accurate changes in body mass over a short period of time, such as during exercise, can be directly attributable to water changes due to sweat. However, weight measurements are easily impacted by changes in clothing or worn equipment, weight loss to urination, consumption of water, beverages, or food. As a result, weight measurements are useful only in short term experiments where other sources of weight variation are controlled.

Spectroscopic measurements of skin hydration (not total body water or hydration) have been reported including NIR (Egawa, Arimoto, Hirao, Takahashi, & Ozaki, 2006) and confocal Raman spectroscopy (Nagakawa, Matsumoto, & Sakai, 2010). The NIR studies were focused on measuring hydration in the stratum corneum and not total body water or hydration. The water reference used in (Egawa, Arimoto, Hirao, Takahashi, & Ozaki, 2006) was capacitance, which provides a measure of water content in the stratum corneum rather than the entire depth of the skin, and the simulations were performed to a depth of 1 mm, so the research is not applicable in the general measurement applications.

(Nagakawa, Matsumoto, & Sakai, 2010) studied dermal water content using confocal Raman spectroscopy. Within this study, they also examined age related changes in skin water content and diurnal changes in water content. According to their research, elderly forearm skin had a much higher water content than younger skin. Also, they determined that dermal water content was significantly higher in the afternoon than in the morning. However, this appears to contradict earlier studies that reported a movement of fluid from the face to the leg from the beginning to the end of the day. From these reports, based on ultrasound measurements, forearm dermal water can be expected to decrease over the course of the day. Consequently, it is not established whether the results from (Nagakawa, Matsumoto, & Sakai, 2010) are accurate.

The existing approaches to measuring hydration are limited by being inaccurate, slow, intrusive, large, expensive, or unreliable. Therefore a method of measuring the hydration of the body that is fast, accurate, non-invasive, small and reliable would be of great benefit. Water and hydration systems based on spectroscopy offer the ability to obviate the limitations of the existing approaches. Spectroscopic devices have been used to measure analytes other than water in biological samples such as human tissue. Some examples include the measurement of glucose and alcohol in the body. While each of these analytes has a different set of considerations and challenges, there is a variety of information in the literature and a number of patents have been filed and granted related to the measurement of blood glucose, alcohol, bilirubin, and oxygen saturation using non-invasive techniques such as absorption spectroscopy, Raman spectroscopy, Kromoscopy, fluorescence spectroscopy, polarimetry, ultrasound, transdermal measurements, photo-acoustic spectroscopy.

### SUMMARY

Although there has been substantial work conducted in attempting to produce commercially viable non-invasive near-infrared spectroscopy-based systems for determination of accurate analyte levels in humans, no such device has been marketed on a wide scale. This is in part due to the complexity of measuring a relatively small concentration of target element (analyte) of interest such as glucose or alcohol within the much more concentrated constituents of skin such as water, collagen, and other naturally occurring components. Interestingly, the available literature on the subject teaches that a major contributing factor to the difficulty of measurement is the presence of water on a large and obscuring scale.

In all of the cases reviewed, water is viewed as an obscuring signal to be removed from the measurement of interest via various techniques and analysis so as to measure the underlying signal of interest. In no case is the concentration of water of interest other than to eliminate its effect on the measurement of interest. Therefore, given the relative abundance of water in a typical skin sample, it should be significantly more feasible to spectroscopically measure either the absolute level of hydration, or at a minimum, a relative level of hydration in the skin relative to measurements of less concentrated analytes such as alcohol or glucose.

Quantitative spectroscopy offers the potential for a completely non-invasive water and/or hydration measurement that is not sensitive to the limitations of the current measurement methodologies. Attributes of interest include, as examples, analyte presence, analyte concentration (e.g., water concentration), direction of change of an analyte concentration, rate of change of an analyte concentration, disease or condition presence (e.g., hydration), disease or condition state, and combinations and subsets thereof. Non-invasive measurements via quantitative spectroscopy are desirable because they are painless, do not require a fluid draw from the body, carry little risk of contamination or infection, do not generate any hazardous waste, and can have short measurement times.

As an example, Robinson et al. in U.S. Pat. No. 4,975,581 disclose a method and apparatus for measuring a characteristic of unknown value in a biological sample using infrared spectroscopy in conjunction with a multivariate model that is empirically derived from a set of spectra of biological samples of known characteristic values. The above-mentioned characteristic is generally the concentration of an analyte but also can be any chemical or physical property of the sample. The method of Robinson et al. involves a two-step process that includes both calibration and prediction steps.

In the calibration step, the infrared light is coupled to calibration samples of known characteristic values so that there is attenuation of at least several wavelengths of the infrared radiation as a function of the various components and analytes comprising the sample with known characteristic value. The infrared light is coupled to the sample by passing the light through the sample or by reflecting the light off the sample. Absorption of the infrared light by the sample causes intensity variations of the light that are a function of the wavelength of the light. The resulting intensity variations at a minimum of several wavelengths are measured for the set of calibration samples of known characteristic values. Original or transformed intensity variations are then empirically related to the known characteristics of the calibration samples using multivariate algorithms to obtain a multivariate calibration model. The model preferably accounts for subject variability, instrument variability, and environment variability.

In the prediction step, the infrared light is coupled to a sample of unknown characteristic value, and a multivariate calibration model is applied to the original or transformed intensity variations of the appropriate wavelengths of light measured from this unknown sample. The result of the prediction step is the estimated value of the characteristic of the unknown sample. The disclosure of Robinson et al. is incorporated herein by reference.

A further method of building a calibration model and using such model for prediction of analytes and/or attributes of tissue is disclosed in U.S. Pat. No. 6,157,041 to Thomas et al., entitled “Method and Apparatus for Tailoring Spectrographic Calibration Models,” the disclosure of which is incorporated herein by reference.

In U.S. Pat. No. 5,830,112, Robinson describes a general method of robust sampling of tissue for non-invasive analyte measurement. The sampling method utilizes a tissue-sampling accessory that is path length optimized by spectral region for measuring an analyte such as water. The patent discloses several types of spectrometers for measuring the spectrum of the tissue from 400 to 2500 nm, including acousto-optical tunable filters, discrete wavelength spectrometers, filters, grating spectrometers and FTIR spectrometers. The disclosure of Robinson is incorporated hereby reference.

Although there has been substantial work conducted in attempting to produce commercially viable systems for determination of hydration, total body water, and/or water concentration, no such device is presently available that meets the needs of many commercial markets. It is believed that prior art systems discussed above have failed for one or more reasons to fully meet the commercial challenges which make the design of a non-invasive hydration measurement system a formidable task. Thus, there is a substantial need for a commercially viable device that incorporates subsystems and methods with sufficient accuracy and precision to make relevant in vivo or ex vivo determinations of water and/or hydration in biological samples such as human tissue.

The present teachings generally relates to the use of quantitative spectroscopy for measuring the presence or concentration of water, hydration levels, hydration state, lactose, lactate, collagen, proteins, or a combination thereof utilizing non-invasive techniques in combination with multivariate analysis.

The present system overcomes the challenges posed by the spectral characteristics of biological samples by incorporating a design that includes, in some embodiments, six optimized subsystems. The design contends with the complexities of the tissue spectrum, high signal-to-noise ratio and photometric accuracy requirements, tissue sampling errors, calibration maintenance problems, calibration transfer problems plus a host of other issues. The six subsystems include an illumination subsystem, a tissue sampling subsystem, a spectrometer subsystem, a data acquisition subsystem, a computing subsystem, and a calibration subsystem.

The present teachings further comprise apparatuses and methods that allow for implementation and integration of each of these subsystems in order to maximize the net attribute signal-to-noise ratio. The net attribute signal is the portion of the measured spectrum that is specific for the attribute of interest because it is orthogonal to all other sources of spectral variance. The orthogonal nature of the net attribute signal makes it perpendicular to the space defined by any interfering species and as a result, the net attribute signal is uncorrelated to these sources of variance. The net attribute signal-to-noise ratio is directly related to the accuracy and precision of the present teachings for non-invasive determination of the attribute by quantitative spectroscopy.

The present teachings can use near-infrared radiation for analysis. Radiation in the wavelength range of 0.7 to 2.5 or 0.75-1.4 microns (or wavenumber range of 14,200 to 4,000 cm⁻¹) can be suitable for making some non-invasive measurements because such radiation has acceptable specificity for a number of analytes, including water, along with tissue optical penetration depths of several millimeters (up to a few cm) with acceptable absorbance characteristics. In the 0.7 to 2.5 micron spectral region, the large numbers of optically active substances that make up the tissue complicate the measurement of any given substance due to the overlapped nature of their absorbance spectra. Multivariate analysis techniques can be used to resolve these overlapped spectra such that accurate measurements of the substance of interest can be achieved. Multivariate analysis techniques, however, can require that multivariate calibrations remain robust over time (calibration maintenance) and be applicable to multiple instruments (calibration transfer). Other wavelength regions, such as the visible and infrared, can also be suitable for the present teachings. Furthermore, in addition to absorption spectroscopy, other methods such as Raman and photoacoustic spectroscopy can be suitable for the present teachings.

The present teachings present a multidisciplinary approach to the design of a spectroscopic instrument that incorporates an understanding of the instrument subsystems, tissue physiology, multivariate analysis, near-infrared spectroscopy and overall system operation. Further, the interactions between the subsystems have been analyzed so that the behavior and requirements for the entire non-invasive measurement device are well understood and result in a design for a commercial instrument that will make non-invasive measurements with sufficient accuracy and precision at a price and size that is commercially viable.

The subsystems of the non-invasive monitor are highly optimized to provide reproducible and, preferably, uniform radiance of the biological sample, low sampling error, depth targeting within the sample (for example tissue layers or locations in the sample that contain the property of interest), efficient collection of spectra from the tissue, high optical throughput, high photometric accuracy, large dynamic range, excellent thermal stability, effective calibration maintenance, effective calibration transfer, built-in quality control, and ease-of-use.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic depiction of a non-invasive hydration measurement device incorporating the subsystems of the present teachings;

FIG. 2 is an alternative schematic depiction of a non-invasive hydration measurement device system incorporating the subsystems of the present teachings;

FIG. 3 is a schematic depiction of a non-invasive hydration measurement device system where the illumination subsystem and spectrometer subsystems have been combined into an illumination/modulation subsystem;

FIG. 4 is a graphical depiction of the concept of net attribute signal in a three-component system;

FIG. 5 is a diagrammed view of an example embodiment of a tungsten filament light source;

FIG. 6 is a diagrammed view of an example embodiment of a ceramic blackbody light source;

FIG. 7 is a diagrammed view of an example embodiment of an igniter light source in an integrating chamber;

FIG. 8 is a diagrammed view of an example embodiment of a combined illumination—sampling subsystem;

FIG. 9 is a diagrammed view of a system of the present teachings using a means for spatially and angularly homogenizing emitted radiation;

FIG. 10 is a schematic of an embodiment of the present teachings incorporating a blackbody light source with Hadamard encoding;

FIG. 11 is a schematic of an embodiment of the present teachings incorporating a blackbody light source with Hadamard encoding, where the encoding is performed after the light has interacted with the sample;

FIG. 12 depicts the various aspects of a sampling subsystem orientation;

FIG. 13 is a diagramed view of the sample interface of a two-channel sampling subsystem;

FIG. 14 is a diagramed view of a sampling subsystem;

FIG. 15 is a perspective view of an ergonomic apparatus for holding the sampling surface and positioning a tissue surface thereon;

FIG. 16 is a plan view of the sampling surface of a tissue sampling subsystem, showing a preferred arrangement of illumination and collection optical fibers;

FIG. 17 is an alternative embodiment of the sampling surface of a tissue sampling subsystem;

FIG. 18 is an alternative embodiment of the sampling surface of a tissue sampling subsystem;

FIG. 19 is a graphical representation showing the benefits of a two-channel sampling subsystem;

FIG. 20 is a diagrammed view of the interface between the sampling surface and the tissue when topical interferents are present on the tissue;

FIG. 21 is an alternative perspective view of an ergonomic apparatus for holding a sampling surface and positioning a tissue surface thereon;

FIG. 22 is a diagrammed view of a positioning device for the tissue relative to a sampling surface;

FIG. 23 is a diagram of an integrated sampling subsystem of the present teachings;

FIG. 24 is a simplified schematic view of a Fourier transform interferometer utilized in the spectrometer subsystem of the present teachings;

FIG. 25 is a depiction of an example interferogram obtained from a Fourier Transform interferometer;

FIG. 26 is a schematic representation of a data acquisition subsystem;

FIG. 27 is an alternative schematic representation of a data acquisition subsystem;

FIG. 28 is a diagram of a hybrid calibration formation process;

FIG. 29 is a schematic representation of a decision process that combines three topical interferent mitigation strategies;

FIG. 30 demonstrates the effectiveness of multivariate calibration outlier metrics for detecting the presence of topical interferents;

FIG. 31 shows normalized NIR spectra of 1300 and 3000 K blackbody radiators over the 100-33000 cm⁻¹ (100-0.3 μm) range;

FIG. 32 shows the measured intensity over time observed for a demonstrative ceramic blackbody light source;

FIG. 33 is an embodiment of an electronic circuit designed to monitor and control the temperature of a solid state light source;

FIG. 34 is an embodiment of an electronic circuit designed to control the drive current of a solid state light source including means for turning the light source on and off;

FIG. 35 is a perspective end view and a detail plan view of a light pipe of the present teachings;

FIG. 36 shows the effective path length versus wavenumber for the sampling subsystem 200 of the noninvasive hydration sensor used in a human hydration study;

FIG. 37 shows the first 3 factors of a PCA decomposition of the spectra obtained from the noninvasive hydration sensor used in the human hydration study;

FIG. 38 shows the pure component spectra of water and collagen overlaid with PCA factor 2;

FIG. 39 shows the scores for the first 3 PCA factors for all study participants;

FIG. 40 shows the correlation between the hydration results obtained from the three multivariate methods for two participants;

FIG. 41 shows the hydration results over time obtained from one participant using the three multivariate methods;

FIG. 42 shows the exercise study results over time for one participant overlaid with weight; and

FIG. 43 is a schematic of the arrangement of illumination and collection fibers at the sample interface for an example embodiment of an optical probe of the present teachings.

FIG. 44 is a schematic of an example embodiment of the present teachings.

FIG. 45 is a schematic of an example embodiment of the present teachings.

FIG. 46 is a schematic of an example embodiment of the present teachings that incorporates a protective window.

FIG. 47 shows a comparison of near infrared spectra of gel samples and in vivo spectra.

FIG. 48 shows a diagram of several devices connected to a centralized server.

FIG. 49 shows a diagram of an approach for connecting multiple facilities to a centralized server.

FIG. 50 shows a configuration of multiple devices connected to a single device which in turn connects to a central server.

FIG. 51 shows a configuration of multiple devices interconnected such that a dedicated central server is not required.

FIG. 52 shows an optical probe design whose tissue interface is configured to interrogate deeper tissues.

FIG. 53 shows an embodiment of 3 channel optical probe that facilitates hydration measurements at multiple tissue depths.

FIG. 54 shows a schematic of the utility of multiple-channel optical probes which shows three illumination channels and a single collection channel.

Corresponding reference numerals indicate corresponding parts throughout the several views of the drawings.

### DETAILED DESCRIPTION

For the purposes of the present teachings, the term “analyte concentration” generally refers to the concentration of an analyte, such as water. The term “analyte property” includes analyte concentration and other properties, such as the presence or absence of the analyte or the direction or rate of change of the analyte concentration, or a biometric, which can be measured in conjunction with or instead of the analyte concentration. While the disclosure generally references water as the “analyte” of interest, other analytes, including but not limited to lactose, lactate, collagen, urea, proteins, and hydration state, hydration level, or any other parameter that is useful in determining a sample or person's hydration state or condition can also benefit from the present teachings. The terms “hydration” and “water” are used as an example analyte of interest; the term is intended to include any analyte that provides information regarding a sample or person's state of hydration and includes cases where multiple analytes are used in conjunction to determine hydration state, hydration level, or water concentration. For the purposes of this teachings, the term “hydration byproducts” and/or “hydration biomarkers” includes the chemicals, byproducts, and biomarkers that are indicative of hydration state within the body and are thus included in the terms “analyte concentration” and “hydration”. The term “hydration state” can also refer to the determination of other parameters or analytes of interest that are subsequently used to determine hydration state. For example, in some embodiments of the present teachings the concentration of water in tissues is determined, the concentration of collagen in tissue is determined, and the water and collagen concentrations are combined (for example, a water to collagen ratio) to determine a result indicative of the hydration state.

In some embodiments, a sensor determines the hydration state of the interrogated tissue or breath. The hydration state of the tissue is then related to the hydration state of the body in a subsequent step. In some embodiments, the hydration state of the body is determined using a conversion factor from the hydration state of the measured tissue. In some embodiments, the conversion factor can be one. In some embodiments, the hydration state of the body can also be determined by incorporating additional information in addition to the estimate of the hydration state of the tissue interrogated. Some examples of such information include, but are not limited to age, gender, height, weight, body temperature, the location of the hydration sensor on the body, and ambient temperature.

The term “biometric” refers to an analyte or biological characteristic that can be used to identify or verify the identity of a specific person or subject. The present teachings address the need for analyte measurements of samples utilizing spectroscopy where the term “sample” generally refers to biological tissue that can be in vivo or ex vivo. The term “subject” generally refers to a person from whom a sample measurement was acquired. The term “subdermal” indicates tissues of any type deeper in the body than the dermis of the skin. Subdermal tissues can better represent the hydration state of the body and are thus preferably interrogated in some embodiments of the present teachings

The terms “solid state light source” or “semiconductor light source” refer to all sources of light, whether spectrally narrow (e.g. a laser) or broad (e.g. an LED) that are based upon semiconductors which include, but are not limited to, light emitting diodes (LED's), vertical cavity surface emitting lasers (VCSEL's), horizontal cavity surface emitting lasers (HCSEL's), quantum cascade lasers, quantum dot lasers, diode lasers, or other semiconductor diodes or lasers. Furthermore, plasma light sources and organic LED's, while not strictly based on semiconductors, are also contemplated in the embodiments of the present teachings and are thus included under the solid state light source and semiconductor light source definitions for the purposes of this disclosure. The term “black body light source” refers to any light source that emits radiation based upon Planck's Law or an approximation of Planck's Law. Some examples of black body light sources are filament lamps, glow bars, ceramic light sources, and passive radiators.

For the purposes of these teachings the term “dispersive spectrometer” indicates a spectrometer based upon any device, component, or group of components that spatially separate one or more wavelengths of light from other wavelengths. Examples include, but are not limited to, spectrometers that use one or more diffraction gratings, prisms, holographic gratings. For the purposes of these teachings the term “interferometric/modulating spectrometer” indicates a class of spectrometers based upon the optical modulation of different wavelengths of light to different frequencies in time or selectively transmits or reflects certain wavelengths of light based upon the properties of light interference. Examples include, but are not limited to, Hadamard transform spectrometers, Fourier transform interferometers, Sagnac interferometers, mock interferometers, Michelson interferometers, one or more etalons, or acousto-optical tunable filters (AOTF's). One skilled in the art recognizes that spectrometers based on combinations of dispersive and interferometric/modulating properties, such as those based on lamellar gratings, are also contemplated with respect to the present teachings.

The teachings make use of “signals,” described in some of the examples as absorbance or other spectroscopic measurements. Signals can comprise any measurement obtained concerning the spectroscopic measurement of a sample or change in a sample, e.g., absorbance, reflectance, intensity of light returned, fluorescence, transmission, Raman spectra, or various combinations of measurements, at one or more wavelengths. Some embodiments make use of one or more models, where such a model can be anything that relates a signal to the desired property. Some examples of models include those derived from multivariate analysis methods, such as partial least squares regression (PLS), linear regression, multiple linear regression (MLR), classical least squares regression (CLS), neural networks, discriminant analysis, principal components analysis (PCA), principal components regression (PCR), discriminant analysis, neural networks, cluster analysis, and K-nearest neighbors. Single or multi-wavelength models based on the Beer-Lambert law are special cases of classical least squares and are thus included in the term multivariate analysis for the purposes of the present teachings.

While it is widely understood that the human body is largely comprised of water, it is important to note that the concentration of water is not uniform throughout the body. Even at rest, different types of tissue (e.g. skin, organs, muscle, etc.) contain different water concentrations at any given moment. Furthermore, when the equilibrium of the body is disrupted (e.g. through exercise, ambient temperature, trauma, or other conditions) two phenomena occur: a net change in the amount of total water in the body (referred to as “total body water” or “TBW”), and redistribution and changes in relative water concentration between the various tissues and compartments within the body. Furthermore, both the total body water and relative concentrations in the tissues and compartments depend on demographics such as age and gender. For example, children have a higher percentage of water (as a function of weight) than adults. A 70 kg adult has approximately 42 liters of total body water of which 28 liters are intracellular and 14 liters are extracellular. Of the 14 liters of extracellular water, 3 liters are in the blood, 1 liter is in the spinal and brain fluid and the remainder is present in interstitial fluid. These quantities are not static since the fluid moves between compartments as needed, and are only meant to approximate relative water distributions in an average adult over time.

“Euhydration” is the term used when the body is in fluid balance. Meanwhile, “dehydration” refers to the state where there is a water deficit and “hyperhydration” refers to water excess in the body. Fluid balance is regulated via a combination of fluid intake and fluid excretion. When total body water is below its optimum, two mechanisms stimulate thirst and thereby encourage fluid intake: baroreceptors are stimulated in response to a decrease in blood volume and pressure, and osmoreceptors are stimulated in response to elevated plasma osmolality (which is caused by a reduction in plasma volume).

Under normal conditions (no exercise, significant temperature change, trauma, etc.), the body regulates water content to within 0.2% of body weight over a 24-hour period despite daily losses of 2-4 liters that must be replaced via fluid consumption. Water loss is regulated primarily through the kidneys where a typical sedentary adult loses approximately 1-2 L a day via urine. Other losses include approximately 450 ml by evaporation through the skin, 250-350 ml by evaporation through respiratory system, and 200 ml via feces. Higher losses are seen during periods of physical activity and high environmental temperatures (mainly due to sweat for thermoregulation).

Based on the natural variation in hydration over time as well as the body's response to exercise, environment, and trauma there is a need to monitor the body's hydration state over time as it can provide useful information regarding the current status of the body and provide immediate indication on the amount of fluid required to replenish the body's TBW. Furthermore, the ability to measure the water concentration in different parts of the body and/or in different compartments within the body can also provide useful diagnostic information regarding the current state of the body and the potential presence of medical conditions that impact fluid transfer within the body. As a result, in some embodiments of the present teachings, the hydration sensor can provide insights into total body water as well as local body water concentration by measuring multiple locations on the body and interrogating different types of tissue. Furthermore, the detection of water and other analytes in breath can be utilized to provide immediate and trend data for TBW.

Regardless of the measurement objective, any successful device must exhibit sensitivity and selectivity for the analyte of interest. Sensitivity is achieved by measuring a signal whose magnitude depends on the amount of the analyte of interest present in the sample. Selectivity is achieved when at least some part of the measured signal is independent of the signals related to other analytes, interferences, and noise. Spectroscopic measurements of analytes achieve sensitivity and selectivity to different degrees depending on the underlying form of spectroscopy used (absorption, emission, etc.), the wavelength region selected (visible, near-infrared, IR, etc.), and the specific embodiments of the measurement device (resolution, number of wavelengths, signal to noise ratio). In the case of near-infrared absorption spectroscopy, sensitivity is achieved via the Beer-Lambert Law that states that the magnitude of an analyte's absorption is dependent upon its concentration. Selectivity is achieved due to the fact that each chemical species has a unique absorption spectrum across multiple wavelengths that are often referred to as a “molecular fingerprint”. Thus, selectivity is achieved by isolating the analyte of interest's signal from the signals of other species and interferences. This is done by measuring a spectrum comprised of multiple wavelengths and subsequently determining the portion of the spectrum that is unique to the analyte of interest (e.g. related to the molecular fingerprint of the analyte). The magnitude of the resulting signal is related to the analyte's concentration.

Different wavelength regions contain different types of information that can influence their relative utility in performing non-invasive spectroscopic determinations of water concentration or hydration. While all wavelength regions could have utility depending on the application it is important to note that some regions are preferable due to a better balance of sensitivity and selectivity for water and hydration measurements. The IR region (2,500 nm to 25,000 nm) is known to have wavelengths where water is a strong absorber. While the strong absorption can be advantageous in some embodiments of the present teachings, it does limit the amount of signal that can be measured. In a transmission measurement where light is introduced to the sample on one side and collected from the other, the strong absorption of water results in total absorption of the introduced light and therefore no signal is measurable from the sample. In reflectance (light is introduced and collected from the same side of the sample), only photons that travel a very short distance through the sample can appreciably exit the sample and be detected. As a result, IR spectroscopy of water containing samples is typically limited to reflectance measurements where a depth of penetration of a few hundred micrometers results in an analytically useful measurement. For example, if epidermal hydration were the property of interest, reflectance measurement of IR wavelengths could be used, as their depth of penetration is consistent with the desired property. However, as epidermal hydration is not strongly representative of total body water or hydration, alternative spectral regions are more useful in order to achieve deeper depth of penetration (and potentially transmission measurements) and therefore interrogate tissues within the body that better relate to total body water and hydration.

The visible region of the electromagnetic spectrum (400-700 nm) can also be useful in some embodiments of the present teachings. Visible measurements have the potential advantages of low noise, small, inexpensive instrumentation that can be useful in some embodiments. However, the visible has two deficiencies that must be taken into consideration. First, the water signal in the visible region is comprised of high-order overtones (harmonics) of the spectral features encountered in the IR. While these signals are related to chemical structure, they are extremely weak (1000's of times weaker) and less defined relative to the signals in the IR region. As a result, visible wavelengths can suffer from poor sensitivity (due to weak signals) and selectivity (due to less spectral definition). Second, spectroscopic measurements of human tissues the visible region can have a significant ethnic dependence due to skin pigmentation. Melanin and other dye's present in the skin contribute their own signals to the spectroscopic measurement. These signals can be much larger in magnitude than the absorbance of water and can impart a strong inter-person and ethnic variation in the spectroscopic measurement. As a result, the presence of skin dyes can impact selectivity and the measurement signal to noise ratio.

The near-infrared (NIR) region (700-2500 nm) lies between the visible and IR regions and exhibits a useful balance of their spectroscopic characteristics that make it the preferred wavelength region for the present teachings. The NIR exhibits little or no signal loss from skin pigments that obviates one of the primary limitations of the visible. Furthermore, the NIR region exhibits water signals with moderate absorptivities that provide a balance of signal strength and path length. As a result, NIR wavelengths enable deeper depth of penetration into tissues, such as muscle tissue, that are more strongly related to total body water and hydration. In some embodiments, the absorptivities of the NIR enable sufficiently long path lengths such that transmission measurements can be achieved.

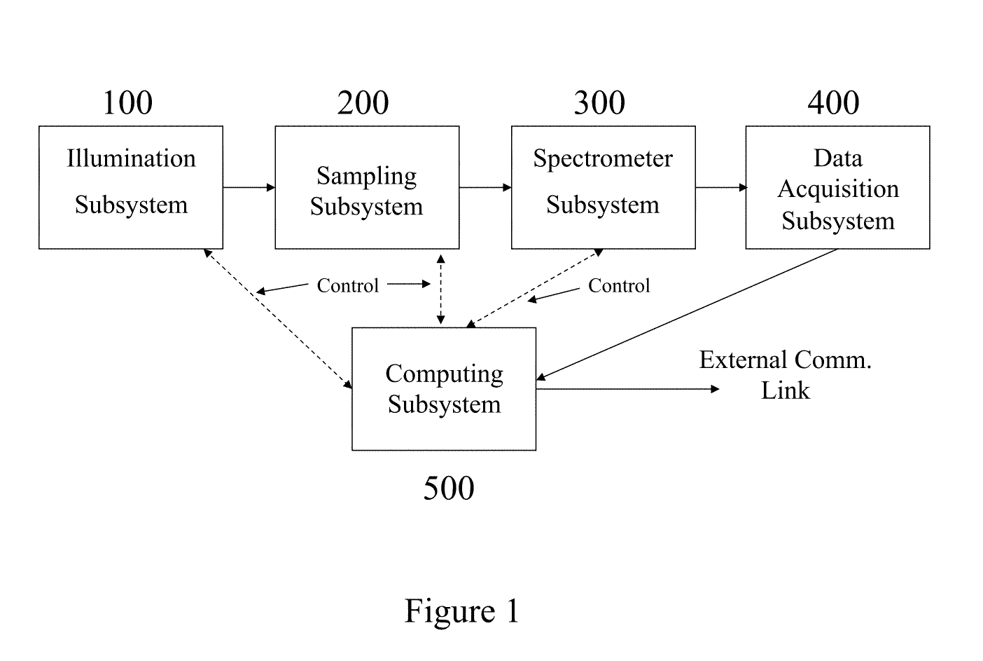

Referring now to FIG. 1, a non-invasive monitor that is able to achieve acceptable levels of accuracy and precision for analyte property measurements is depicted in schematic view. The overall systems of the present teachings can be viewed for discussion purposes as comprising five subsystems; those skilled in the art will appreciate other subdivisions of the functionality disclosed. The subsystems include an illumination subsystem 100 (also referred to as an “illuminator”), a sampling subsystem 200 (also referred to as an “optical receiver”), a spectrometer subsystem 300, a data acquisition subsystem 400, a processing subsystem 500, and a calibration subsystem (not shown). In some embodiments, the functions of the data acquisition subsystem 400 are combined with those of the processing subsystem 500 and are collectively referred to as a processing subsystem.

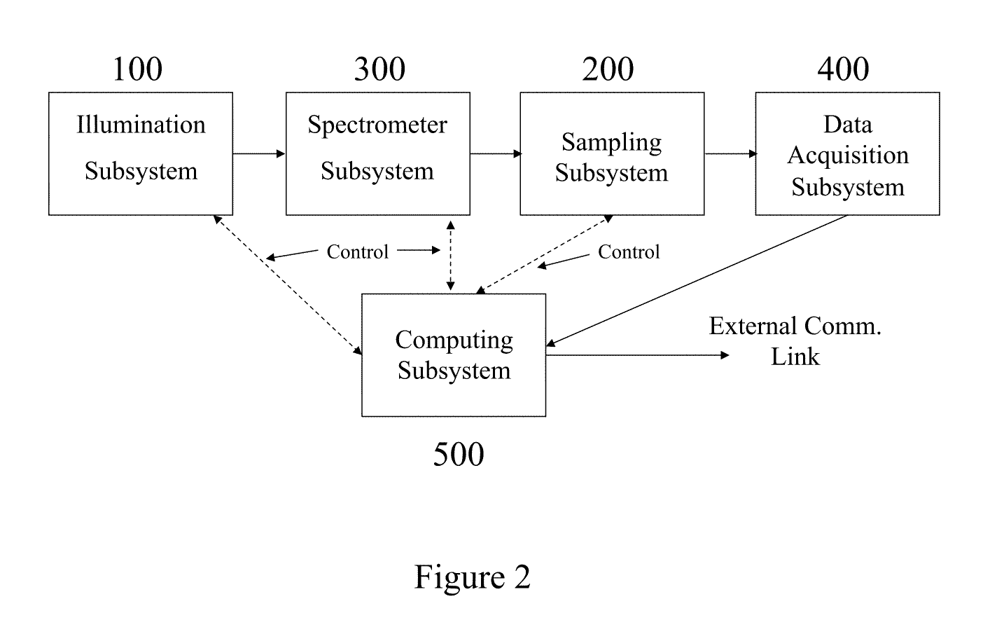

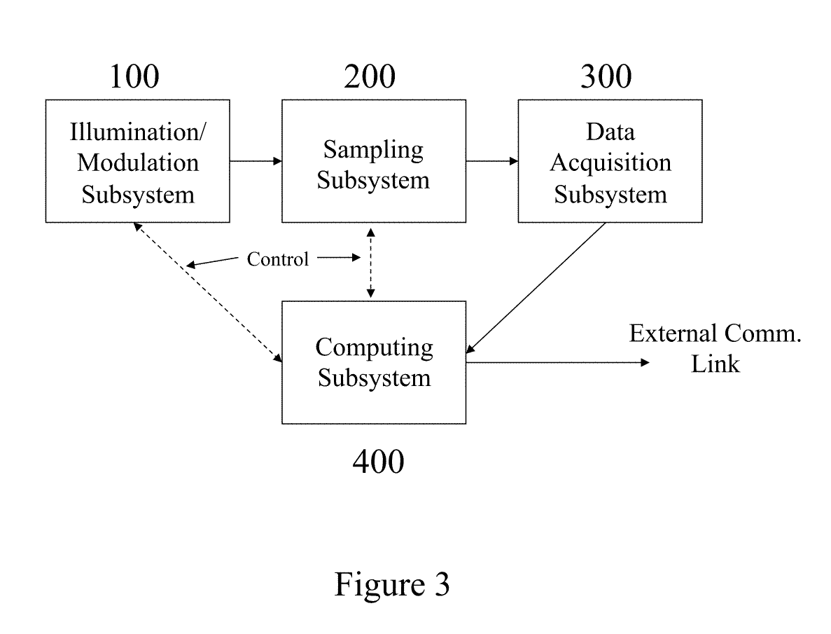

The specific orientation of the six subsystems can be altered in order to optimize the balance between the net attribute signal to noise ratio, form factor, and other important parameters such as ruggedness and cost. For example, FIG. 2 shows an alternative orientation of the subsystems in which the sampling (200) and spectrometer (300) subsystems have been interchanged. FIG. 3 shows an alternative orientation where the light source (100) and spectrometer (300) subsystems have been combined into a single “illumination/modulation” subsystem. The following discussion focuses on the case where the sampling subsystem precedes the spectrometer subsystem (FIG. 1). The subsequent discussion is not meant to preclude alternative arrangements or combinations of the subsystems.

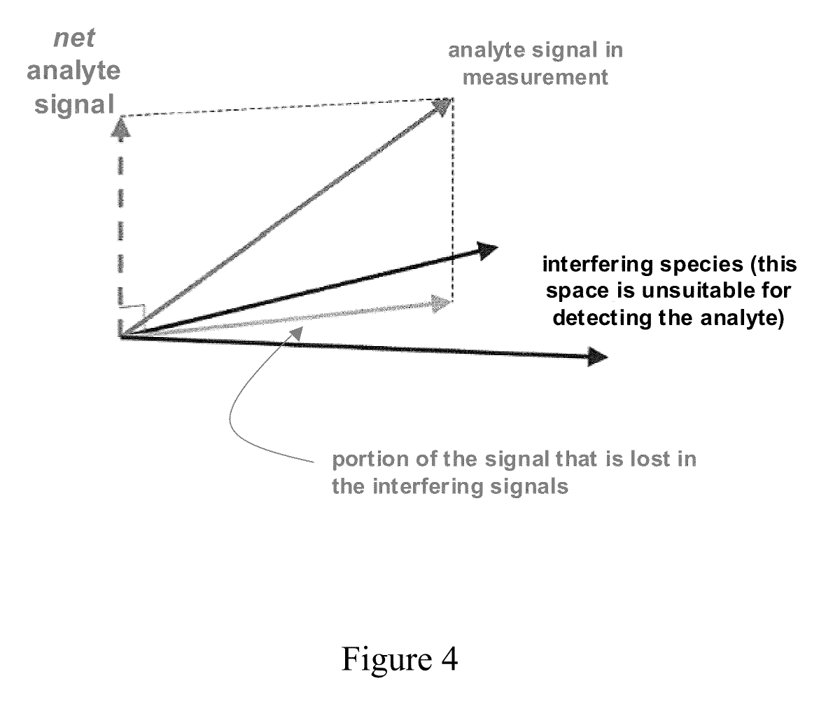

The subsystems can be designed and integrated in order to achieve a desirable net attribute signal-to-noise ratio. The net attribute signal is the portion of the near-infrared spectrum that is specific for the attribute of interest because it is orthogonal to other sources of spectral variance. FIG. 4 is a graphical representation of the net attribute signal in a three dimensional system. The net attribute signal-to-noise ratio is directly related to the accuracy and precision of the non-invasive attribute determination by quantitative near-infrared spectroscopy with the present teachings.

The subsystems provide reproducible and preferably spatially uniform radiance of the tissue, low tissue sampling error, depth targeting of appropriate layers of the tissue, efficient collection of diffuse reflectance spectra from the tissue, high optical throughput, high photometric accuracy, large dynamic range, excellent thermal stability, effective calibration maintenance, effective calibration transfer, built-in quality control and ease-of-use. Each of the subsystems is discussed below in more detail.

The purpose of the illumination subsystem is to provide light in the desired wavelength region or regions for use by the remainder of the noninvasive measurement system. In some cases, the light source can be a single element that emits many wavelengths simultaneously, a single element that emits only one wavelength, multiple individual elements that each emits a single wavelength, or a combination thereof. The specific type of light source or sources used in embodiments of the present teachings depend on the wavelength region of interest, the type and design of subsequent subsystems, and the environment in which the noninvasive hydration monitor will be used. Some examples of suitable types of light sources include, but are not limited to, blackbody light sources, light emitting diodes (LED's), fluorescent tubes or lamps, solid state lasers (diode lasers, DFB's, VCSEL's, etc.), gas lasers, filament lamps, ceramic radiators, or any other means for generating light in the desired wavelength regions.

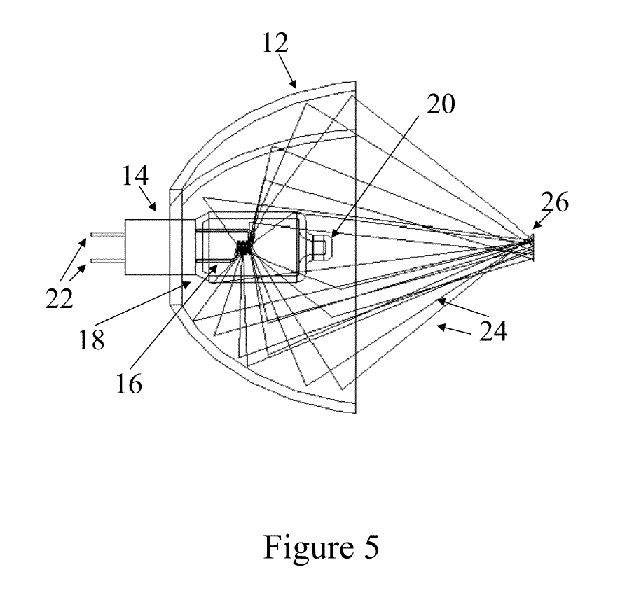

In some embodiments, the illumination subsystem 100 generates the near-infrared (NIR) light used to interrogate the tissue of a human. The illumination subsystem contains a broadband, polychromatic light source 14 that emits radiation in the NIR portion of the spectrum. The light source 14 can also emit radiation outside of the NIR. An example of a suitable light source 14 is a 40-watt, 22.8-volt tungsten filament lamp (FIG. 5). The light source 14 can be driven by a tightly regulated power supply. The power supply can supply the light source with constant current, constant voltage, or constant power. The power supply for the light source can provide tight regulation of current, voltage, or power in order to keep the color temperature and emissivity of the light source as stable as possible. Fluctuations of the light source's color temperature and emissivity can be a source of noise in the measurement and can reduce the net attribute signal and, subsequently, the accuracy and precision of the measurement.

In some embodiments, the overall system of the present teachings includes a power supply that provides regulated, low noise power to all of the subsystems. The power supply can include a soft start function that extends the useful life of the light source by eliminating startup transients and limiting the current required to initially power the light source.

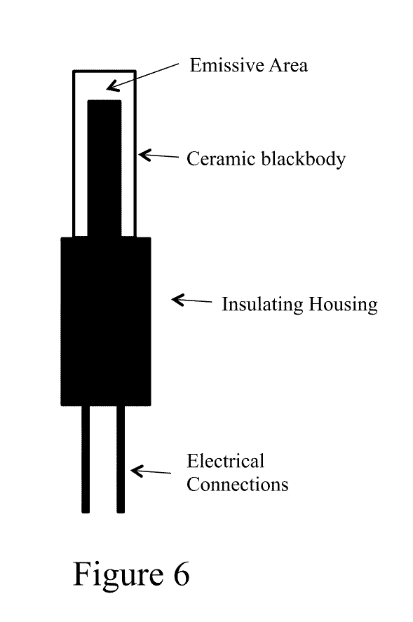

Another example light source is a resistive element such as those commonly used as igniters for furnaces and stoves. These light sources have a lower color temperature than standard filament lamps and are therefore more efficient in the near-infrared spectral region. These sources also have comparatively large emissive surfaces that are less sensitive to spatial effects that are encountered throughout the lifetime of the light source. An example of a suitable resistive element light source is a 24-watt, SiN ceramic blackbody light source (FIG. 6). An additional advantage of igniter-based light sources is a substantially longer lifetime when compared to filament lamps.

Solid state light sources such as, but not limited to, light emitting diodes (LED's) and diode lasers (e.g. Pabry-Perot, DFB, VCSEL, HCSEL, and/or quantum cascade lasers) are also suitable light sources for the present teachings. Several parameters of systems for measuring analyte properties incorporating solid state light sources must be considered including, but not limited to, the number of solid-state light sources required to perform the desired measurement, the emission profile of the light sources (e.g. spectral width, intensity), light source stability and control, and their optical combination. As each light source is a discrete element, it can be advantageous to combine the output of multiple light sources into a single beam such that they are consistently introduced and collected from the sample.

Another advantage of solid-state light sources is that many types (e.g. diode lasers, VCSEL's, quantum cascade lasers) emit a narrow range of wavelengths (which in part determines the effective resolution of the measurement). Consequently, in preferred embodiments, shaping or narrowing the emission profile of light sources with optical filters or other approaches is not required as they are already sufficiently narrow. This can be advantageous due to decreased system complexity and cost. Furthermore, the emission wavelengths of some solid state light sources, such as diode lasers and VCSEL's, are tunable over a range of wavelengths via either the supplied drive current, drive voltage, or by changing the temperature of the light source. The advantage of this approach is that if a given measurement requires a specific number of wavelengths, the system can achieve the requirement with fewer discrete light sources by tuning them over their feasible ranges. For example, if measurement of a noninvasive property required 20 wavelengths, 10 discrete lasers can be used with each of the being tuned to 2 different wavelengths during the course of a measurement. In this type of scheme, a Fourier or Hadamard approach remains appropriate by changing the modulation frequency for each tuning point of a light source or by combining the modulation scheme with a scanning scheme.

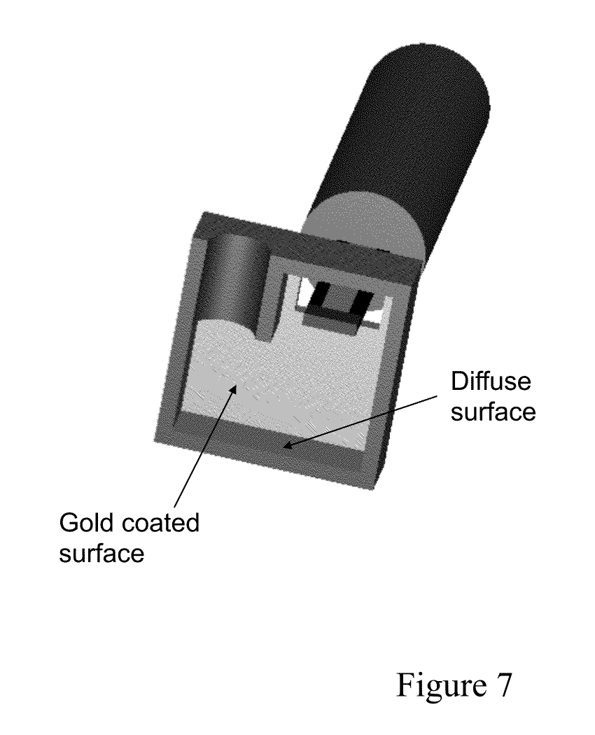

In addition to the light source and regulated power supply, the illumination subsystem can contain optical elements 12, 13, 90 that collect the radiation from the light source and transfer that light to the input of the sampling subsystem (200) or spectrometer subsystem (300), depending on the system orientation. The elements that make up the transfer optics can include collimating and/or condensing optics, optical filters, optical diffusers, a reflective integrating chamber, a diffuse integrating chamber, a homogenizer or light pipe for scrambling and the corresponding mechanical components to hold the optics and light source. FIG. 7 is a diagramed view of an embodiment of the illumination subsystem where an igniter light source is enclosed in an integrating chamber. In this embodiment, the chamber serves as means for collecting light as well as spatially and angularly homogenizing the light prior to introduction of the light to the rest of the system.

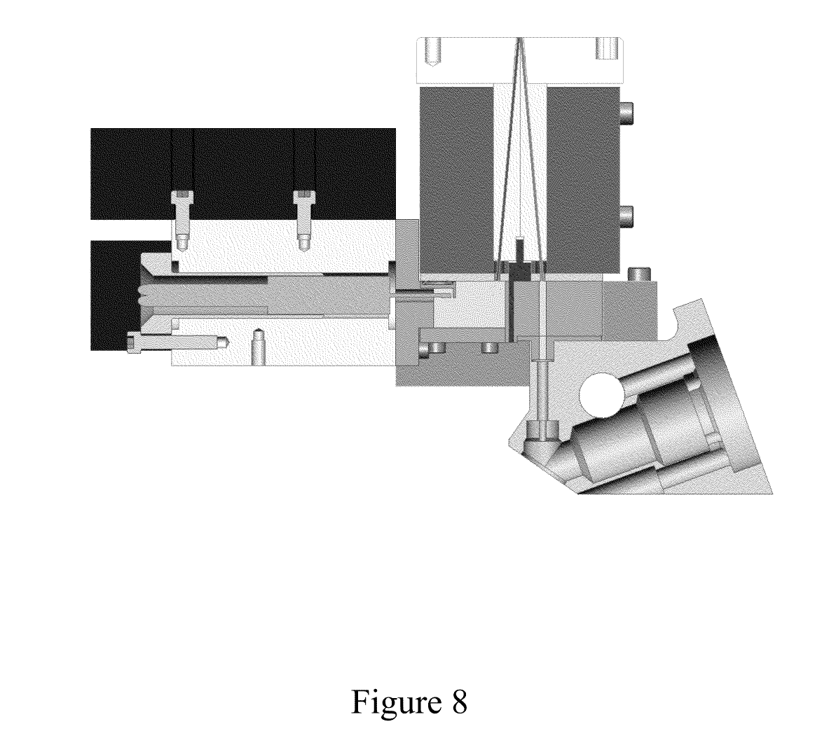

In some embodiments, the illumination subsystem can also contain the optical elements that deliver light to the tissue. In these embodiments, the illumination subsystem can be considered a part of the tissue sampling subsystem. In this case, the number of overall optical components can be reduced which can result in a reduced cost, an improvement in optical efficiency, and smaller physical size. FIG. 8 is a graphical representation of a preferred embodiment where the illumination subsystem has been incorporated into the tissue sampling subsystem.

The collimating optics can be refractive or reflective elements. A lens is an example of a refractive collimating optic. A parabolic mirror is an example of a reflective collimating optic. The condensing optics can also be refractive or reflective. A lens is an example of a refractive condensing optic. An elliptical mirror is an example of a reflective condensing optic. Suitable materials for lenses and mirrors are known in the art. The reflective optics can have a smooth finish, a rough finish or a faceted finish depending on the configuration of the illumination subsystem. The rough or faceted finishes for the reflective optics destroy the coherence of the light source image to create a more uniform radiance pattern. The refractive optics can be spherical or aspherical. A Fresnel lens, a special type of aspherical lens, also can be employed. The collimating and/or condensing optics collect radiation from the source and transfer the radiation to the input of the sampling subsystem 200 or to other optical elements that perform additional operations on the light before it is passed to the sampling subsystem 200.

One or more optical filters can be employed to preferentially pass radiation only in the spectral region of interest. The optical filter can be one or a combination of long pass, short pass, or band pass filters. These filters can be absorptive, interference, or dichroic in nature. In some embodiments, the optical filters are anti-reflection coated to preserve the transmittance of light in the spectral region of interest. These filters can also perform spectral shaping of the radiation from the light source to emphasize certain portions of the spectrum over others. The optical filtering can bandlimit the radiation passed to the rest of the system and increase the SNR in the region of interest and to keep from burning or otherwise damaging the tissue of the subject. Bandlimiting the radiation improves the net attribute signal by reducing Shot noise that results from unwanted radiation outside the spectral region of interest.

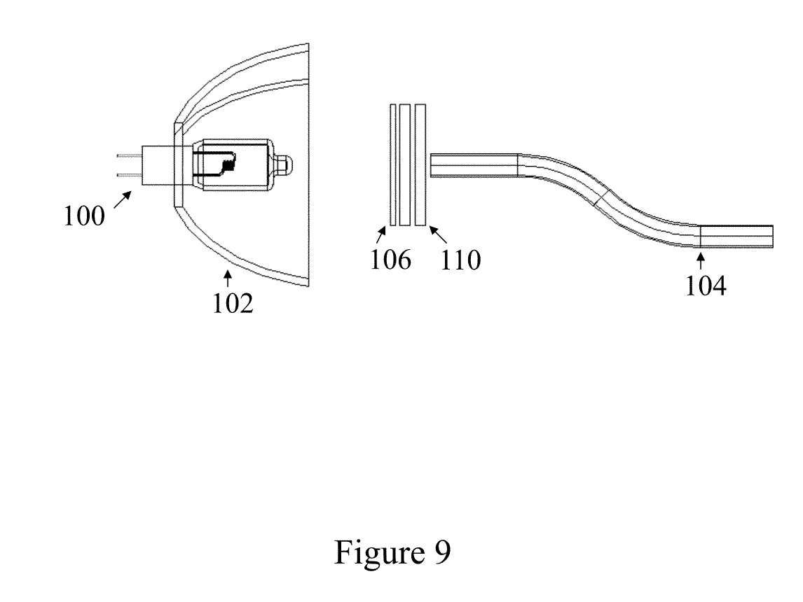

The optical diffusers 13 and scramblers 90 in the illumination subsystem provide reproducible and, preferably, uniform radiance at the input of the sampling subsystem 200 or spectrometer subsystem, depending on the system orientation. Uniform radiance can ensure good photometric accuracy and even illumination of the sample. Uniform radiance can also reduce errors associated with manufacturing differences between light sources. Uniform radiance can be utilized in the present teachings for achieving accurate and precise measurements. FIG. 9 is a diagramed view of an embodiment of the illumination subsystem where a filament lamp is used in conjunction with an optical diffuser and scrambler in order to provide uniform radiance at the input of the sampling subsystem. See, e.g., U.S. Pat. No. 6,684,099, incorporated herein by reference.

A ground glass plate is an example of an optical diffuser. The ground surface of the plate effectively scrambles the angle of the radiation emanating from the light source and its transfer optics. A light pipe can be used to scramble the intensity of the radiation such that the intensity is spatially uniform at the output of the light pipe. In addition, light pipes with a double bend will scramble the angles of the radiation. For creation of uniform spatial intensity and angular distribution, the cross section of the light pipe should not be circular. Square, hexagonal and octagonal cross sections are effective scrambling geometries. The output of the light pipe can directly couple to the input of the tissue sampler or can be used in conjunction with additional transfer optics before the light is sent to the tissue sampler. See, e.g., U.S. patent application Ser. No. 09/832,586, “Illumination Device and Method for Spectroscopic Analysis,” incorporated herein by reference.

In some embodiments of the present teachings, the illumination subsystem (100) and the spectrometer subsystem (300) can be combined into a single subsystem (referred to as an “illumination/modulation subsystem”, see FIG. 3) that can offer significant advantages. Similar to the illumination subsystem 100, the illumination/modulation subsystem 100 generates the light used to interrogate the sample (e.g. skin tissue of a human). In classical spectroscopy using dispersive or interferometric spectrometers, the spectrum of a polychromatic light source (or sample of interest) is measured either by dispersing the different wavelengths of light spatially (e.g. using a prism or a diffraction grating) or by modulating different wavelengths of light to different frequencies (e.g. using a Michelson interferometer). In these cases, a spectrometer (a subsystem distinct from the light source) is required to perform the function of “encoding” different wavelengths either spatially or in time such that each can be measured substantially independently of other wavelengths. While dispersive and interferometric spectrometers are known in the art and can adequately serve their function in some environments and applications, they can be limited by their cost, size, fragility, and complexity in other applications and environments.

An advantage of solid-state light sources incorporated in some embodiments of the present teachings is that they can be modulated in intensity. Thus, multiple light sources that emit different wavelengths of light can be used with each light source modulated at a different frequency. The independently modulated light sources can be optically combined into a single beam and introduced to the sample. A portion of the light can be collected from the sample and measured by a single photodetector. The result is the effective combination of the light source and the spectrometer into a single illumination/modulation subsystem that can offer significant benefits in size, cost, energy consumption, and overall system stability since the spectrometer, as an independent subsystem, is eliminated from the measurement system.

The modulation scheme for the light sources must also be considered as some types of sources can be amenable to sinusoidal modulations in intensity where others can be amenable to being switched on and off or square wave modulated. In the case of sinusoidal modulation, multiple light sources can be modulated at different frequencies based on the electronics design of the system. The light emitted by the multiple sources can be optically combined, for example using a light pipe or other homogenizer, introduced and collected from the sample of interest, and then measured by a single detector. The resulting signal can be converted into intensity versus wavelength spectrum via a Fourier, or similar, transform.

Alternatively, some light sources are switched between the on and off state or square wave modulated which are amenable to a Hadamard transform approach. However, in some embodiments, rather than a traditional Hadamard mask that blocks or passes different wavelengths at different times during a measurement, the Hadamard scheme can be implemented in electronics as solid state light sources can be cycled at high frequencies. A Hadamard or similar transform can be used to determine the intensity versus wavelength spectrum.

It is important to note that the present teachings also envision several embodiments illumination/modulation subsystems that incorporate blackbody light sources rather than solid-state light sources. In these embodiments, the broad blackbody source is converted to multiple, narrow light sources using optical filters such as, but not limited to, linearly variable filters (LVF's), dielectric stacks, distributed Bragg gratings, photonic crystal lattice filters, polymer films, absorption filters, reflection filters, etelons, dispersive elements such as prisms and gratings, and quantum dot filters. The resulting multiple bands of wavelengths can be modulated by a Fourier scheme or Hadamard mask. Similar to the solid-state concepts, the spectrometer system is combined with the light source that can offer substantial benefits in terms of size, cost, and the robustness of the system.

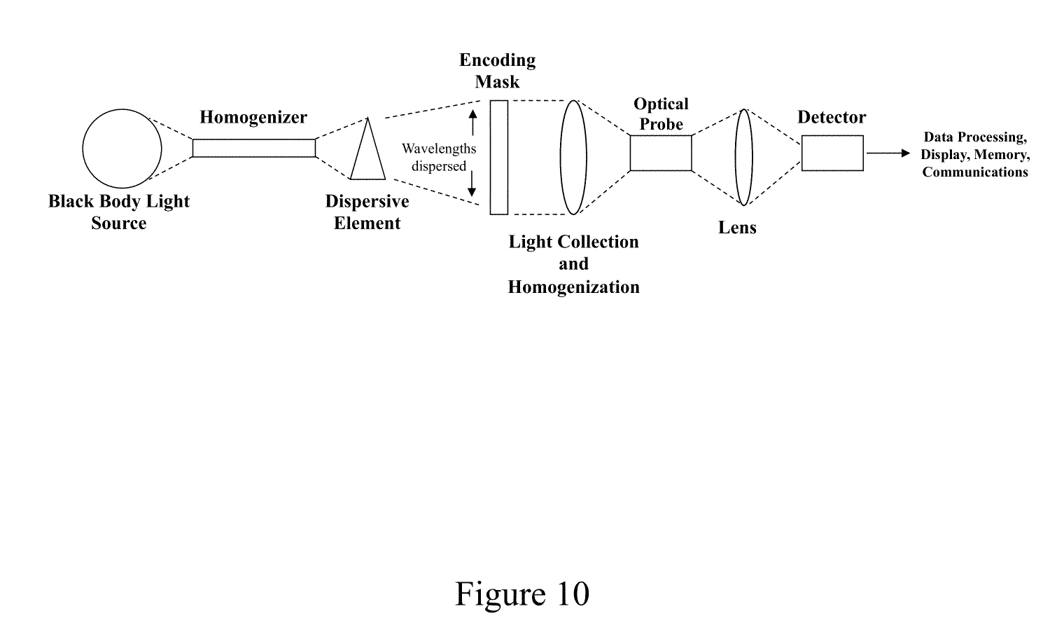

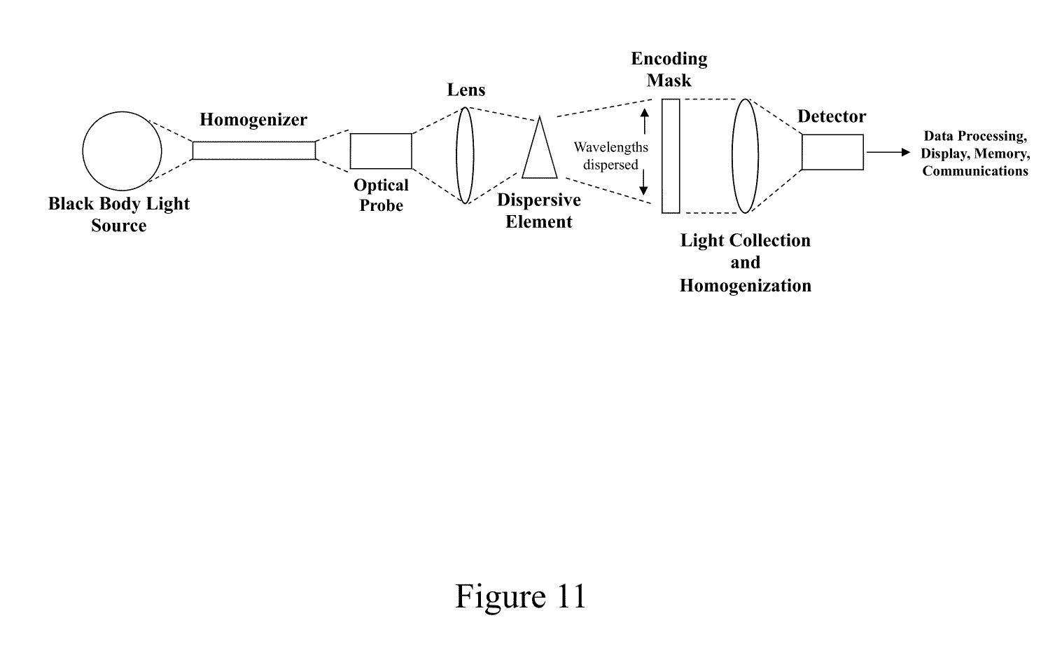

In other embodiments, a dispersive element such as a grating or prism is used to spatially separate the wavelengths of light from a broadband source (either a blackbody, LED, or other broad emitting light source). The dispersive element separates the different wavelengths that can be independently modulated at their locations on a focal plane using a Hadamard mask or mechanical chopper (e.g. for a Fourier scheme). Similar to the embodiments previously described, the resulting light can be homogenized and introduced to the optical probe. FIGS. 10 and 11 show schematics of embodiments of the present teachings that incorporate a blackbody light source with Hadamard encoding.

In mechanically modulated embodiments incorporating a Hadamard mask or mechanical chopper, in some cases it can be advantageous to perform the modulating step after the light has been collected from the sample by the optical probe (200). FIG. 11 shows a schematic of an embodiment of such a system.

The sampling subsystem 200 has two primary purposes. First, the sampling system 200 delivers light to, and collects light from, the sample. The sampling subsystem 200 can accomplish this via measurements in transmission, measurements in reflectance, or a combination thereof. Second, the sampling system 200 is designed such that it provides control over where the light propagates while within the sample. For example, in reflectance measurements it can be advantageous to design the sampling subsystem 200 to preferentially interrogate specific depths within the sample and thereby accentuate the contribution of those depths to the measured signals. Additional considerations of the sampling subsystem 200 are efficiency in order to maximize signal to noise ratio, and a design consistent with measuring the type of sample, or location on the sample, under consideration.

In the case of noninvasive hydration measurement systems of the present teachings the sampling subsystem 200 is preferably designed such that it can interrogate one or more locations on the human body. Some suitable locations for the purposes of the present teachings are the finger, arm, forearm, upper arm, shoulder, lip, ear, ear lobe, leg, face, or any location on the body that is accessible or useful to determining the water concentration, total body water, and/or hydration state of the person being tested.

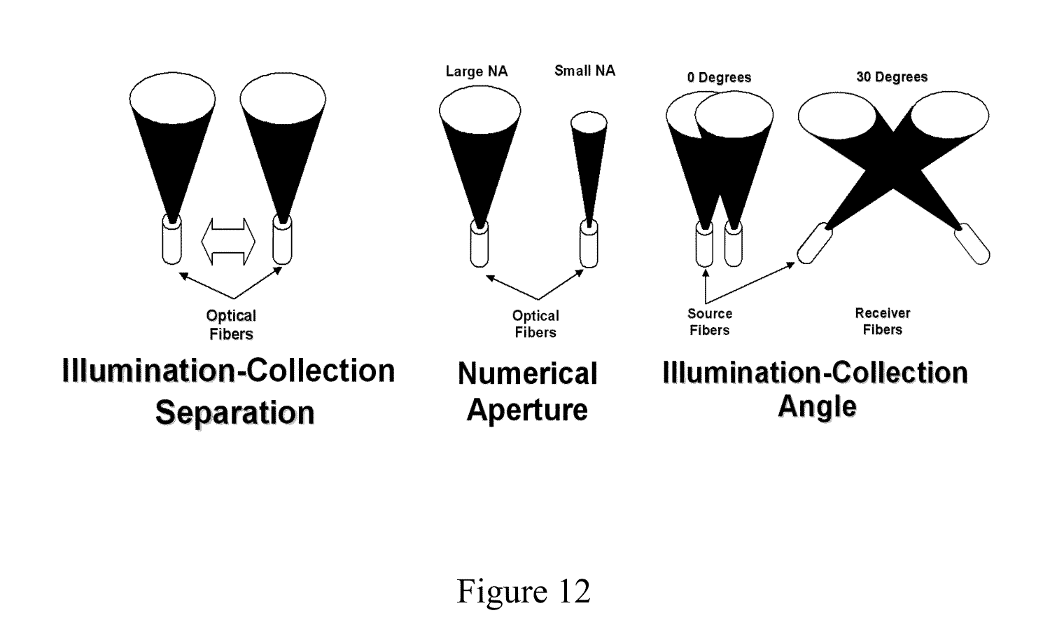

The sampling subsystem can also use one or more channels, where a channel refers to a specific orientation of the illumination and collection fibers. An orientation is comprised of the angle of the illumination fiber or fibers, the angle of the collection fiber or fibers, the numerical aperture of the illumination fiber or fibers, the numerical aperture of the collection fiber or fibers, and the separation distance between the illumination and collection fiber or fibers. FIG. 12 is a diagram of parameters that form an orientation. Multiple channels can be used in conjunction, either simultaneously or serially, to improve the accuracy of the noninvasive measurements.

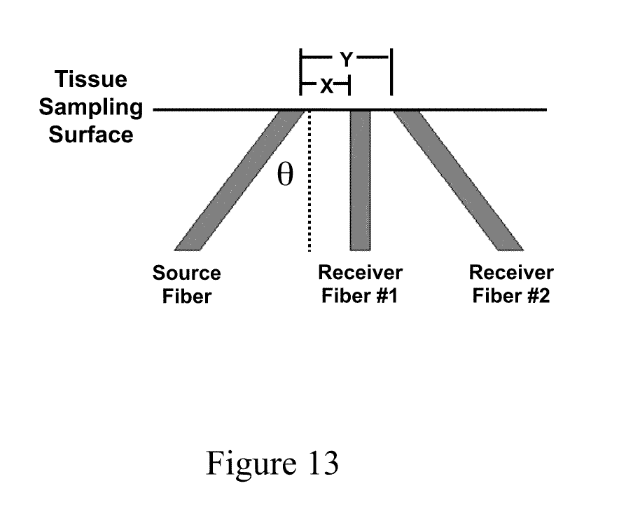

FIG. 13 is a diagram of a two channel sampling subsystem. In this example, the two channels are measuring the same tissue structure. Therefore each channel provides a measurement of the same tissue from a different perspective. The second perspective helps to provide additional spectroscopic information that helps to decouple the signals due to scattering and absorption.

Referring to FIG. 13, the group of fibers (1 source, 1 receiver #1, and 1 receiver #2 in this example) can be replicated 1 to N times in order to increase the sampler area, interrogate a larger physical area of the sample, and improve optical efficiency. Each of the fibers in an orientation can have a different numerical aperture and angle (θ). The distances between fibers, X and Y, determine the source-receiver separation. Furthermore, an additional source channel can be added that creates a 4-channel sampling subsystem. One skilled in the art recognizes the large number of possible variants on the number and relationship between channels.

An important aspect of the present teachings and the sampling subsystem design is the ability to target specific depths within the sample. Depth targeting can be accomplished by the choice of the wavelengths used in the noninvasive hydration measurement system as wavelengths that are absorbed less strongly by the sample can propagate longer distances without strong attenuation. For example, 1,000 nm light can travel much further in human tissue than 2,500 nm light due to the greatly reduced absorptivities of water and collagen at shorter wavelengths. For a fixed sampling subsystem 200 design, shorter wavelengths result in more light penetrating deeper into the sample prior to being collected and delivered to the spectrometer subsystem 300.

Depth targeting can also be accomplished through the design of the one or more channels in the sampling subsystem 200. For example, increasing the physical separation between illumination and collection optical fibers strictly forces longer path lengths through the sample. Furthermore, inclining the illumination and collection towards each other decreases path lengths. Similarly, smaller numerical apertures (narrower illumination and collection cones) eliminate shallow light trajectories. In summary, the wavelengths of light used for analysis and the sampling subsystem 200 design are both useful in determining the depths that a given embodiment of the present teachings will interrogate.

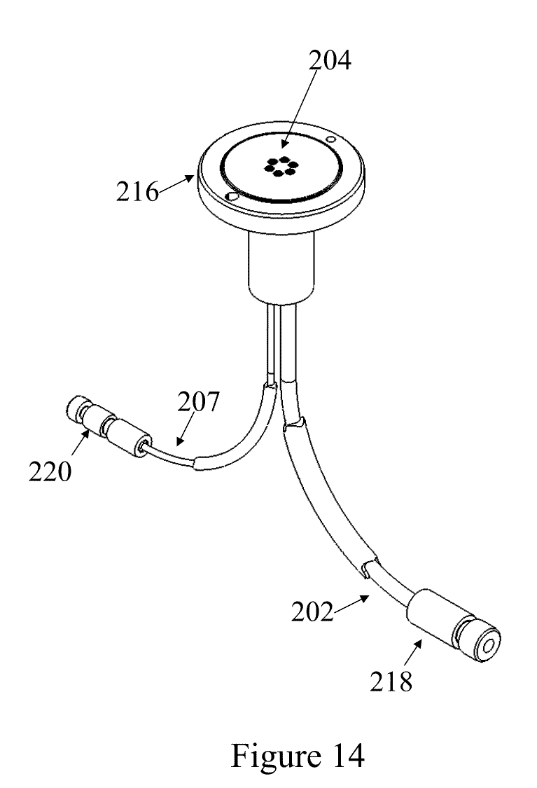

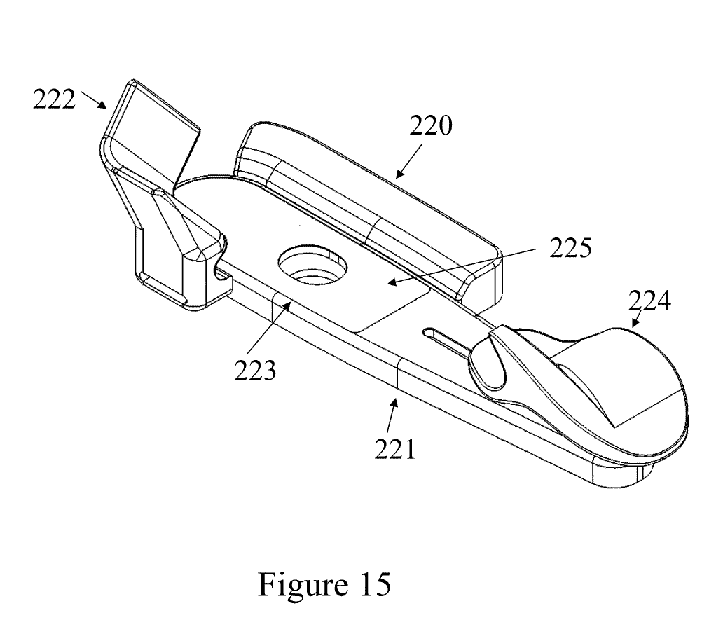

Referring to FIG. 1, the sampling subsystem 200 introduces radiation generated by the illumination subsystem 100 into the tissue of the subject, collects a portion of the radiation that is not absorbed by the tissue and sends that radiation to the spectrometer subsystem 300. Referring to FIG. 14, the tissue sampling subsystem 200 has an optical input 202, a sampling surface 204 which forms a tissue interface 206 that interrogates the tissue and an optical output 207. The subsystem further includes an ergonomic apparatus 210, depicted in FIG. 15, which holds the sampling surface 204 and positions the tissue at the interface 206. In a preferred subsystem, a device that thermostats the tissue interface is included and, in some embodiments, an apparatus that repositions the tissue on the tissue interface in a repetitive fashion is included. In other embodiments, an index matching fluid can be used to improve the optical interface between the tissue and sampling surface. The improved interface can reduce error and increase the efficiency, thereby improving the net attribute signal. See, e.g. U.S. Pat. Nos. 6,622,032, 6,152,876, 5,823,951, and 5,655,530, incorporated herein by reference.

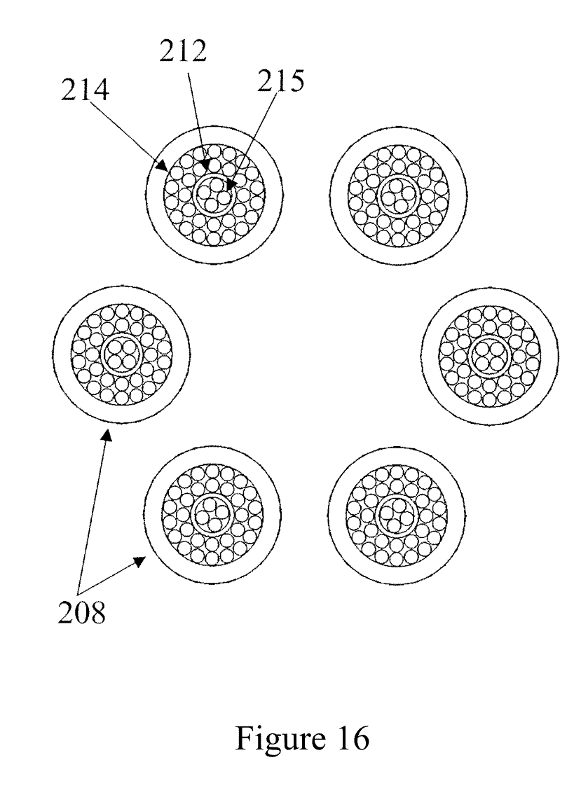

The optical input 202 of the tissue sampling subsystem 200 receives radiation from the illumination subsystem 100 (e.g., light exiting a light pipe) and transfers that radiation to the tissue interface 206. As an example, the optical input can comprise a bundle of optical fibers that are arranged in a geometric pattern that collects an appropriate amount of light from the illumination/modulation subsystem. FIG. 16 depicts one example arrangement. The plan view depicts the ends of the input and output fibers in a geometry at the sampling surface including six clusters 208 arranged in a circular pattern. Each cluster includes four central output fibers 212 that collect diffusely reflected light from the tissue. Around each grouping of four central output fibers 212 is a cylinder of material 215 that ensures about a 100 μm gap between the edges of the central output fibers 212 and the inner ring of input fibers 214. The 100 μm gap can be important to measuring ethanol in the dermis. As shown in FIG. 16, two concentric rings of input fibers 214 are arranged around the cylinder of material 215. As shown in one example embodiment, 32 input fibers surround four output fibers.

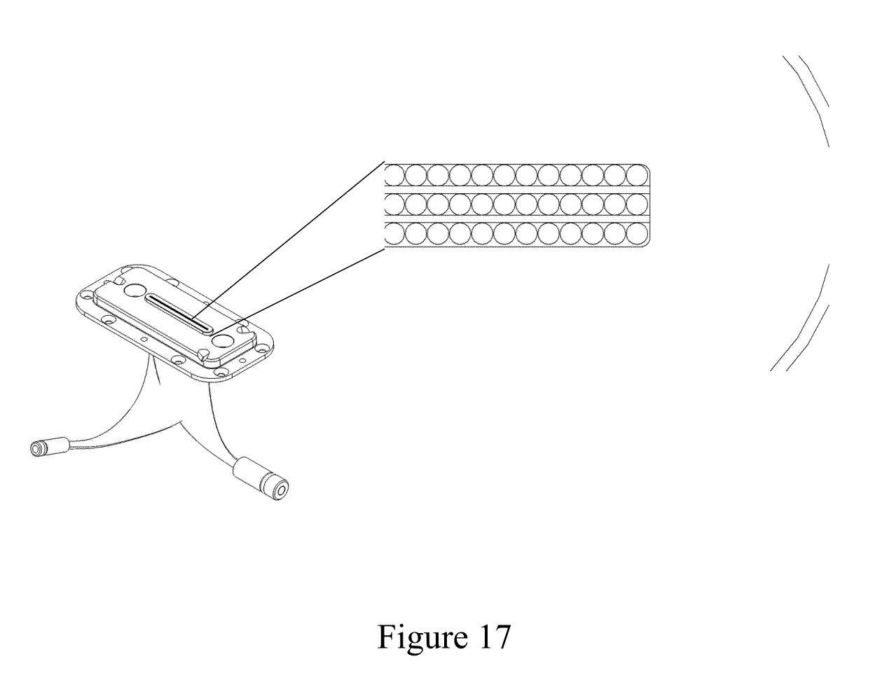

FIG. 17 demonstrates an alternative to cluster geometries for the sampling subsystem. In this embodiment, the illumination and collection fiber optics are arranged in a linear geometry. Each row can be either for illumination or light collection and can be of any length suitable to achieve sufficient signal to noise. In addition, the number of rows can be 2 or more in order to alter the physical area covered by the sampling subsystem. The total number of potential illumination fibers is dependent on the physical size of emissive area of the light source and the diameter of each fiber. Multiple light sources can be used to increase the number of illumination fibers. The number of collection fibers depends upon the area of the interface to the interferometer subsystem. If the number of collection fibers results in an area larger than the interferometer subsystem interface allows, a light pipe or other homogenizer followed by an aperture can be used to reduce the size of the output area of the sampling subsystem. The purpose of the light pipe or other homogenizer is to ensure that each collection fiber contributes substantially equally to the light that passes through the aperture.

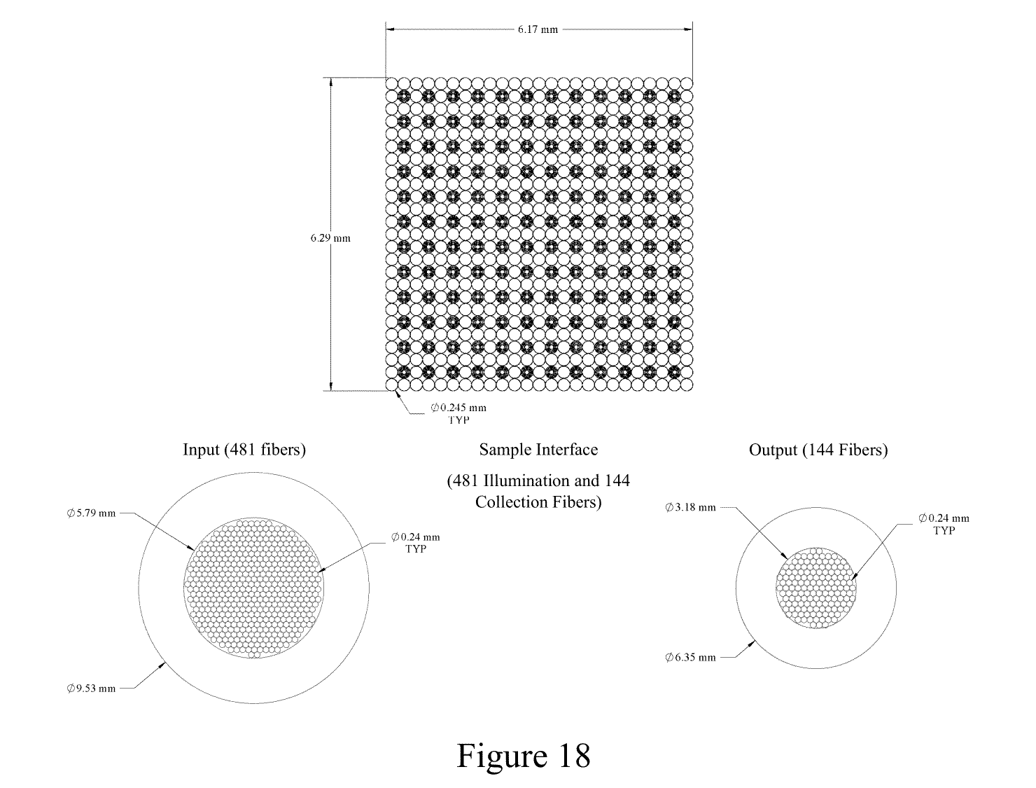

In some embodiments the sampling subsystem of the present teachings, the portion of the optical probe that interacts with the sample can be comprised of a stack of two or more linear ribbons of optical fibers. These arrangements allow the size and shape of the optical probe interface to be designed appropriately for the sample and measurement location (e.g. hand, finger) of interest. FIG. 18 shows an example embodiment of a sampling subsystem based on a linear stack off ribbons. Additional details regarding suitable embodiments for use in the present teachings can be found in co-pending U.S. patent application Ser. Nos. 12/185,217 and 12/185,224, each of which is incorporated herein by reference.

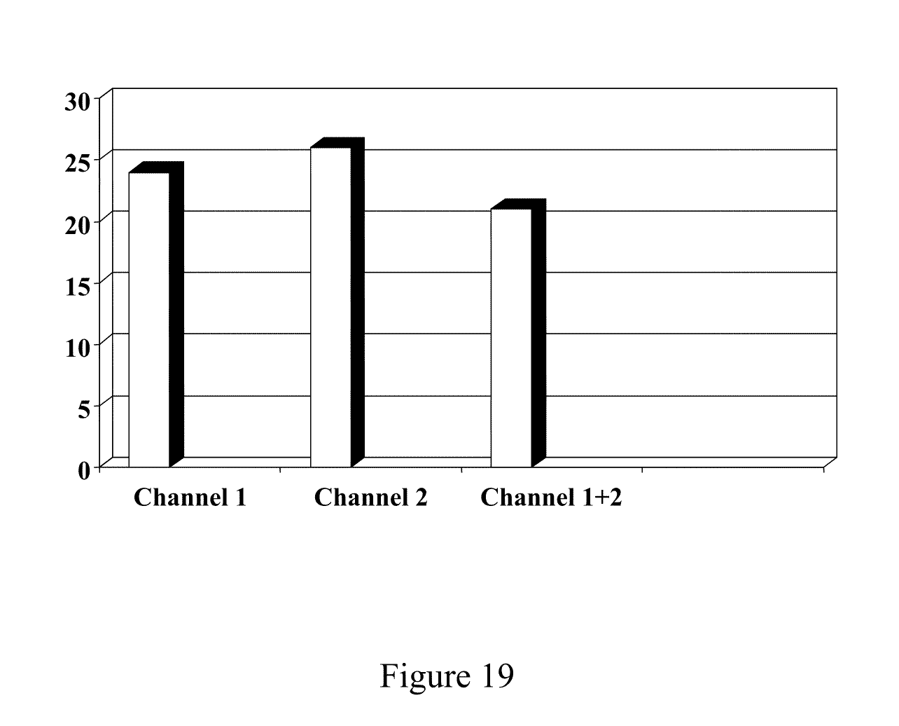

FIG. 19 is a bar chart of example of the benefits of a multiple channel sampler that was used for noninvasive analyte measurements. It is clear from the figure that the combination of the two channels provides superior measurement accuracy when compared to either channel individually. While this example uses two channels, additional channels can provide additional information that can further improve the measurement. An additional aspect of the use of multiple channels is the ability to depth target in a manner that is more effective than a single channel. One channel can be configured to have a deeper penetration while the other is shallower. As deeper penetrating light has to pass through the shallow portions of the sample, the signal obtained from second (shallow) channel can be used to remove the contribution of the shallower layers from the deeper penetrating channel, thereby further accentuating the deeper tissues in the measured signal.

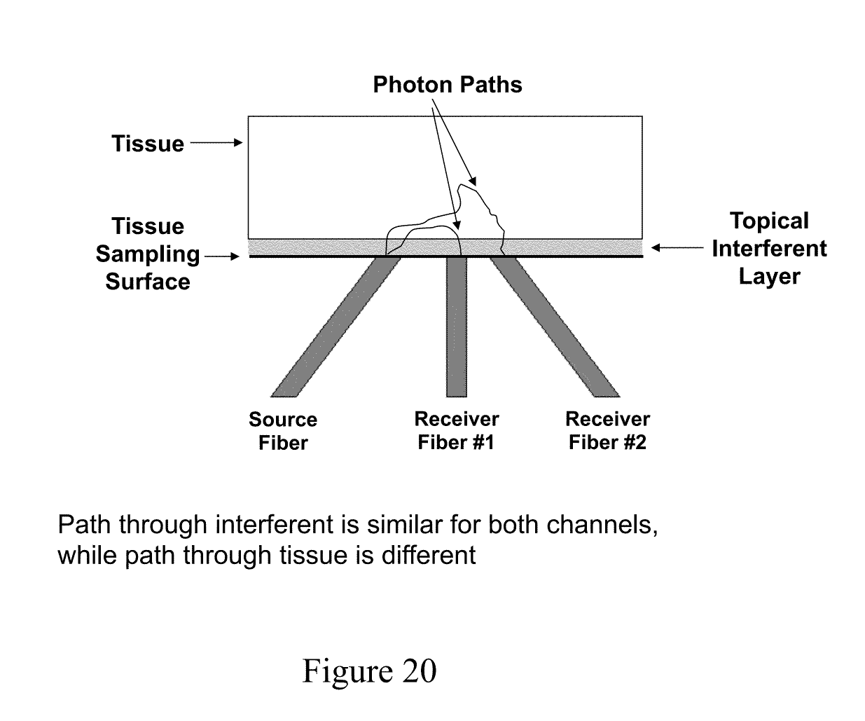

Another aspect of a multiple channel sampling subsystem is the ability to improve detection and mitigation of topical interferents, such as sweat or lotion, present on the sample. FIG. 20 is a diagram of the multiple channel sampling subsystem in the presence of a topical interferent. The figure shows the sampling subsystem at the tissue interface, a layer of topical interferent, and the tissue. In this example the contribution to each channel's measurement due to the topical interferent is identical. This allows the potential to decouple the common topical interferent signal present in both channels from the tissue signal that will be different for the two channels.

The clustered input and output fibers are mounted into a cluster ferrule that is mounted into a sampling head 216. The sampling head 216 includes the sampling surface 204 that is polished flat to allow formation of a good tissue interface. Likewise, the input fibers are clustered into a ferrule 218 connected at the input ends to interface with the illumination/modulation subsystem 100. The output ends of the output fibers are clustered into a ferrule 220 for interface with the data acquisition subsystem 300.

Alternatively, the optical input can use a combination of light pipes, refractive and/or reflective optics to transfer input light to the tissue interface. It can be important that the input optics of the tissue sampling subsystem collect sufficient light from the illumination subsystem 100 and from the sample in order to achieve an acceptable net attribute signal.

The tissue interface irradiates the tissue in a manner that targets the compartments of the tissue pertinent to the attribute of interest, and can discriminate against light that does not travel a significant distance through those compartments. As an example, a 100-μm gap discriminates against light that contains little attribute information. In addition, the tissue interface can average over a certain area of the tissue to reduce errors due to the heterogeneous nature of the tissue. The tissue sampling interface can reject specular and short pathlength rays and it can collect the portion of the light that travels the desired pathlength through the tissue with high efficiency in order to maximize the net attribute signal of the system. The tissue-sampling interface can employ optical fibers to channel the light from the input to the tissue in a predetermined geometry as discussed above. The optical fibers can be arranged in pattern that targets certain layers of the tissue that contain good attribute information.

The spacing, angle, numerical aperture, and placement of the input and output fibers can be arranged in a manner to achieve effective depth targeting. In addition to the use of optical fibers, the tissue-sampling interface can use a non-fiber based arrangement that places a pattern of input and output areas on the surface of the tissue. Proper masking of the non-fiber based tissue-sampling interface ensures that the input light travels a minimum distance in the tissue and contains valid attribute information. Finally, the tissue-sampling interface can be thermo-regulated to control the temperature of the tissue in a predetermined fashion. The temperature of the tissue sampling interface can be set such that the teachings reduces prediction errors due to temperature variation. Further, reference errors are reduced when building a calibration model. These methods are disclosed in U.S. patent application Ser. No. 09/343,800, entitled “Method and Apparatus for Non-Invasive Blood Analyte Measurement with Fluid Compartment Equilibration,” which is incorporated herein by reference.

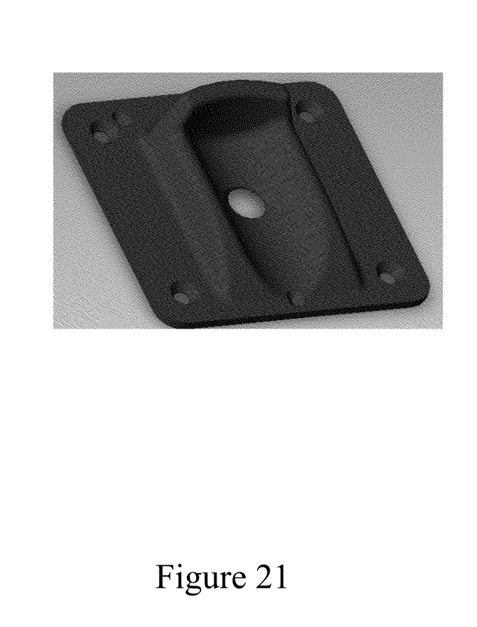

The tissue sampling subsystem can employ an ergonomic apparatus or cradle 210 that positions the tissue over the sampling interface 206 in a reproducible manner. Example ergonomic apparatuses 210 are depicted in FIGS. 15 and 21. An ergonomic cradle design can be essential to ensure good contact between the sample and the sampling interface in some embodiments. The ergonomic cradle 210 includes a base 221 having an opening 223 there through. The opening is sized for receiving the sample head 216 therein to position the sampling surface 204 generally coplanar with an upper surface 225 of the base 221. The ergonomic cradle 210 generally references a part of the sample such that it accurately positions the sample on the sampling interface. Careful attention must be given to the ergonomics of the sampling interface or significant sampling error can result.

The example ergonomic cradle 210 is configured such that the desired location on the subject is reliably located over the sampling head 216. In some embodiments of the present teachings that measure the dorsal forearm of a person, the bracket 222 forms an elbow rest that sets the proper angle between the upper arm and the sampling head 216, and also serves as a registration point for the arm. The adjustable hand rest 224 is designed to hold the fingers in a relaxed manner. The hand rest position is adjusted for each subject to accommodate different forearm lengths. In some embodiments, a lifting mechanism is included which raises and lowers the cradle periodically during sampling to break and reform the tissue interface. Reformation of the interface facilitates reduction of sampling errors due to the rough nature and heterogeneity of the skin. Alternate sites, for example fingertips, can also be accommodated using variations of the systems described herein.

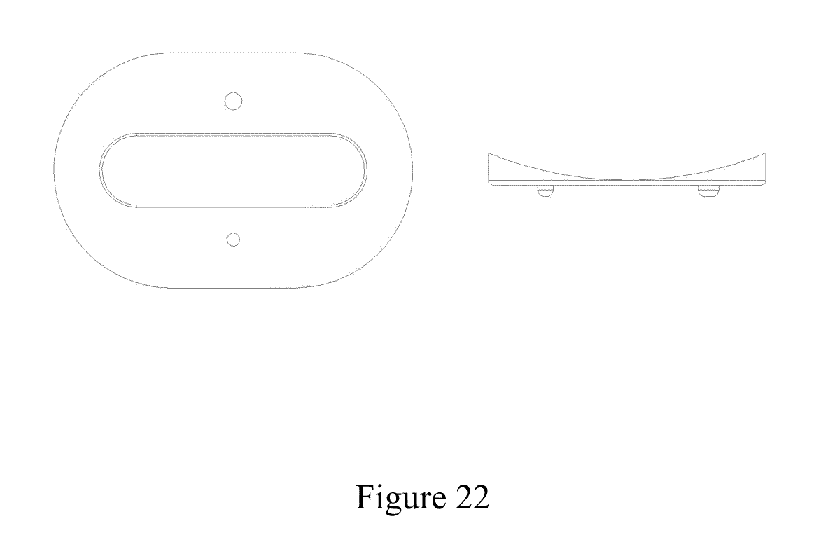

An alternative to the ergonomic cradle is diagramed in FIG. 22. Instead of a cradle located on the measurement system, the positioning device is located on the tissue. The positioning device can either be reusable or disposable and can be adhered to the tissue with medical adhesive. The positioning device can also include an optically transparent film or other material that prevents physical contact with the sampling subsystem while preserving the desired optical characteristics of the measurement. The positioning device interfaces to the sampling subsystem in a pre-determined manner, such as alignment pins, in order to reproducibly locate the tissue to the sampling subsystem. The positioning device also prevents movement of the tissue relative to the sampling subsystem during the measurement process.

The output of the sampling subsystem 200 transfers the portion of the light not absorbed by the tissue that has traveled an acceptable path through the tissue to the spectrometer subsystem 300. The output of the sampling subsystem 200 can use any combination of refractive and/or reflective optics to focus the output light into the spectrometer subsystem 300. In some embodiments, the collected light is homogenized (see U.S. Pat. No. 6,684,099, Apparatus and Methods for Reducing Spectral Complexity in Optical Sampling, incorporated herein by reference) in order to mitigate for spatial and angular effects that might be sample dependent.

In some embodiments of the present teachings, the sampling subsystem 200 does not incorporate optical fibers and alternative means are used to collect light from the light source subsystem 100, deliver it to the sample in a manner sufficient to control the light interaction with said sample, and collect light from the sample and deliver it to the spectrometer subsystem 300 to fiber optics. For example, a reflective chamber can be used to collect light from the light source, homogenize the collected light, and deliver it to the sample. A second chamber can then be used to collect light from the sample, homogenize the collected light, and deliver it to the spectrometer subsystem 300. The orientation of the two chambers relative to each other serves to control the portion of light collected from the sample (e.g. depth of penetration of the light). FIG. 8 shows an embodiment of a chamber-based sampling subsystem 200. While the preceding discussion of chamber-based sampling subsystems 200 were based on a system orientation as shown in FIG. 1, one skilled in the art recognizes that the above mentioned chamber-based light sampling subsystems are equivalently useful in the system orientations shown in FIG. 2 and FIG. 3.

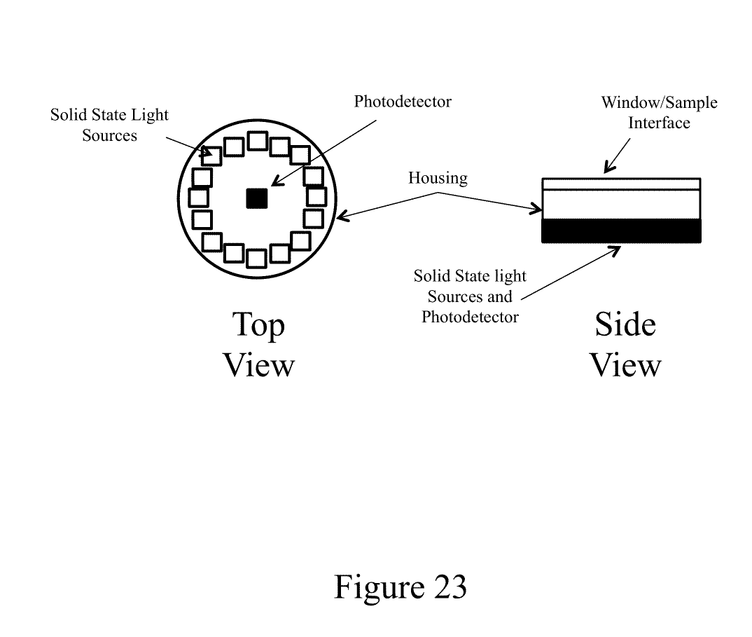

In other embodiments of the present teachings, the photodetector (part of the data acquisition subsystem 400), solid state light sources (part of the illumination subsystem 100), or a combination thereof can be integrated into sampling subsystem 200 (referred to as an “integrated sampling subsystem”. Such approaches can reduce system size, cost, and complexity as well as improve optical efficiency due to a reduction in the number of optical components in the system. In a preferred embodiment shown in FIG. 23, the illumination/modulation subsystem 100 (a special case of the illumination subsystem 100 described above) is integrated into the sampling subsystem 200 along with the photodetector (from the data acquisition subsystem 400). The result of this combination is a very compact system with no need for optical fibers or other means to convey light between the different subsystems of the noninvasive analyte measurement system. The spacing between the photodetector and the solid state light sources serves to control the depth of penetration for the light collected by the photodetector.

Furthermore, in some embodiments different wavelengths (via different solid state light sources) can be at different separations to the photodetector, which allows wavelength (or wavelength band) specific tuning of depth of penetration. In addition, as with optical fiber based sampling subsystems 200, multiple channel sampling subsystems 200 are easily achieved in integrated sampling subsystems by placing additional solid-state light sources at different spacings from the photodetector. As solid-state light sources can individually be powered on and off, selection of each channel for measurement is straightforward. An additional advantage of integrated sampling subsystems is the ability to weight the contribution of different wavelengths or wavelength bands to the measured spectra by increasing or decreasing the number of light sources of a given wavelength relative to those of other wavelengths. One skilled in the art recognizes the advantages and flexibility of the integrated sampling subsystem of the present teachings and recognizes the large number of variations contemplated herein.

The purpose of the spectrometer subsystem 300 is to encode, modulate, or spatially separate different wavelengths of light from each other in order to enable subsequent determination of a spectrum (e.g. an intensity versus wavelength). Different types of spectrometer subsystems can be used to achieve this purpose. A dispersive spectrometer can be used to spatially separate different wavelengths of light. A dispersive element within the spectrometer such as, but not limited to, a reflective or transmission grating, or a prism is used to reflect or refract different wavelengths to a different spatial location at the spectrometer's output (often called a “focal plane”). The intensity is then measured at each desired point in the focal plane in order to obtain the spectrum. The intensities can be measured sequentially using a single element detector and rotating the dispersing element to place the desired wavelength on the detector. Alternatively, a multi-element detector such as a CCD or photodiode array can be used to detect multiple wavelengths simultaneously.

An interferometer, in contrast, modulates each wavelength to a different frequency while, in many cases, leaving their spatial distribution unchanged. The signal measured is then converted to a spectrum via a Fourier or similarly appropriate mathematical transform. Interferometers can exhibit significant advantages relative to dispersive spectrometers including, but not limited to, the multiplex and throughput advantages known in the art. An interferometer is used in some of the preferred embodiments of the present teachings and will be discussed in more detail.

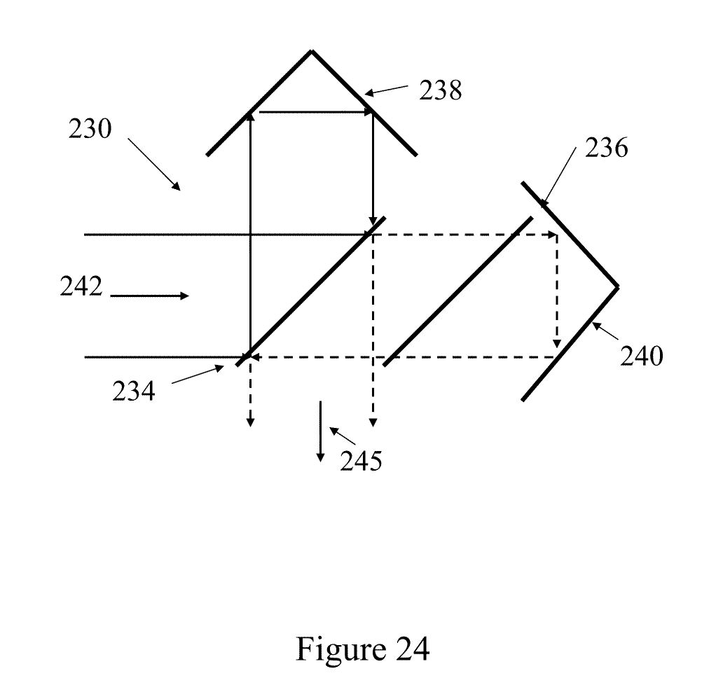

The spectrometer subsystem 300 includes a spectrometer 230 that modulates the sufficiently collimated light from the sampling subsystem 200 to create an interferogram that is received by the detector that is part of the data acquisition subsystem. The interferogram is formed by modulating the wavelengths of light collected by the sampling subsystem 200 or the illumination subsystem 100, depending on the system's orientation, to different frequencies. FIG. 24 schematically depicts one embodiment of a spectrometer 230, called a Fourier Transform interferometer (FTIR), which includes a beamsplitter 234 and compensator optics 236, a fixed retro-reflector 238 and a moving retro-reflector 240. The collimated input light 242 impinges on the beamsplitter optic 234 and is partially reflected and partially transmitted by the coating on the back surface of the beamsplitter 234. The reflected light passes back through the beamsplitter optic 234 and reflects off the fixed retro-reflector 238 and back to the beamsplitter 234. The transmitted light passes through the compensator optic 236 and reflects off the moving retro-reflector 240 and back to the beamsplitter 234. The transmitted and reflected portions of the light recombine at the beamsplitter to create an interference pattern or interferogram. The amount of constructive and/or destructive interference between the transmitted and reflected beams is dependent on the spectral content of the collimated input beam 242 and on the optical path difference between the fixed retro-reflector 238 and the moving retro-reflector 240. One skilled in the art recognizes that there are many types of interferometer architectures and many specific embodiments of each type. All are equally suitable for embodiments of the present teachings and the preceding example is meant to serve as an example of one suitable spectrometer subsystem. For example, in the preceding example, the modulation is achieved by the moving mirror. In alternative embodiments, both mirrors can have a fixed location and the modulation can be achieved by rotating the compensator element or another transmissive or reflective optical element placed in one leg of the interferometer.

A reference laser can allow knowledge of the actual optical path difference as a function of time. Using the knowledge of the optical path difference, the infrared signal can be sampled in equal position increments to satisfy the requirements of a Fourier transform. Typically, a helium neon (HeNe) laser is used as the reference in interferometers because of its comparatively small size and cost relative to other gas lasers. A lower cost, solid-state alternative to HeNe lasers is also suitable. See, e.g., VCSEL patent.

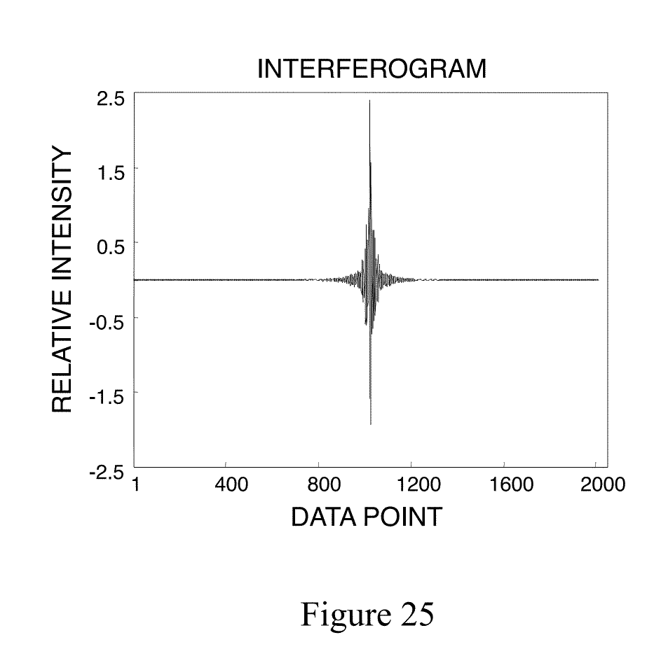

FIG. 25 shows a typical interferogram created by an FTIR spectrometer. At the point of zero path difference between the transmitted and reflected beams, there will be maximum constructive interference, and the centerburst of the interferogram is created. The interferogram is then focused onto a detector (part of the data acquisition subsystem), as shown in FIG. 1. The detector converts the optical interferogram into an electrical representation of the interferogram for subsequent digitizing by the data acquisition subsystem 400.

In an embodiment, the spectrometer subsystem 300 utilizes a Fourier Transform interferometer 230 manufactured by Bomem. This spectrometer utilizes a single plate that contains beamsplitter and compensator functions. In addition, cube corners are used as the end mirrors and both cube corners are moved on a wishbone suspension to create the optical path difference and the subsequent interference record. The Bomem WorkIR™ FTIR spectrometer achieves the desired thermal stability and spectral complexity performance necessary for making non-invasive analyte measurements with NIR spectroscopy. The Fourier Transform interferometer modulates the collimated light from the sampling subsystem 200 to encode the NIR spectrum into an interferogram. The spectral resolution of the interferogram can be in the range of 2 to 64 wavenumbers. The preferred range of spectral resolution is 16-32 wavenumbers. The interferometer will produce either a single-sided or a double-sided interferogram, with the double-sided interferogram being preferred because it achieves a higher net attribute signal and reduces sensitivity to phase errors. The resulting interferogram is preferably passed to a condensing lens 244 and this lens focuses the light onto the detector. The condensing lens 244 is a double convex design with each surface being aspherical in nature. In some embodiments, the lens material can be ZnSe, silicon, or fused silica.

A Fourier Transform interferometer can achieve high SNR and photometric accuracy. In the art, there are many variants of the Michelson interferometer design depicted in FIG. 24. An example interferometer design is disclosed in U.S. patent application Ser. No. 09/415,600, filed Oct. 8, 1999, entitled “Interferometer Spectrometer with Reduced Alignment Sensitivity,” the disclosure of which is incorporated herein by reference. The Fourier Transform interferometer has throughput advantages (Jaquinot and Fellget advantages) relative to dispersive spectrometers and acousto-optical tunable filters. In addition to high throughput, the use of a reference laser in the Fourier Transform interferometer gives the device excellent wavelength axis precision. Wavenumber or wavelength axis precision can be important for effective calibration maintenance and calibration transfer.

The Fourier Transform interferometer subsystem 300 must achieve certain minimum performance specifications for thermal stability, spectral complexity and modulation efficiency. In real world use of the present teachings, ambient temperature and relative humidity can vary with time. Over an ambient temperature operating range of 10° C. to 35° C., the Fourier Transform interferometer must maintain suitable modulation efficiency, for example 50% or better. Modulation efficiency is a measure of the useful signal produced by the FTIR spectrometer and is calculated by taking the ratio of the peak interferogram value at zero path difference to the DC value and then multiplying by 100. The maximum theoretical value of modulation efficiency is 100% with typical Fourier Transform interferometer achieving values in the range of 60% to 95%. Fourier Transform interferometer with modulation efficiencies below 50% have relatively poorer SNR because of the additional Shot noise from the larger proportion of non-signal bearing DC light falling on the photodetector.

In some example embodiments, the Fourier Transform interferometer's change in percent transmittance (% T) can be kept to no more than 1% per degree Celsius. This temperature sensitivity can preserve the analyte net analyte SNR and to simplify calibration maintenance.

Spectroscopic measurement systems typically require some means for resolving and measuring different wavelengths of light in order to obtain a spectrum. As previously discussed, some common approaches achieve the desired spectrum include dispersive (e.g. grating and prism based) spectrometers and interferometric (e.g. Fourier Transform, Michelson, Sagnac, or other interferometer) spectrometers. Noninvasive measurement systems that incorporate such approaches can be limited by the expensive nature of dispersive and interferometric devices as well as their inherent size, fragility, and sensitivity to environmental effects. In some embodiments of the present teachings, an alternative approach for resolving and recording the intensities of different wavelengths is provided that uses solid state light sources such as light emitting diodes (LED's), vertical cavity surface emitting lasers (VCSEL's), horizontal cavity surface emitting lasers (VCSEL's), diode lasers, quantum cascade lasers, other solid state light sources, or a combination thereof. In these embodiments, the light source subsystem 100 and spectrometer subsystem 300 are combined into an illumination/modulation subsystem (see FIG. 3) that is discussed in more detail within the Special Case: Illumination/Modulation Subsystem (100) section of this disclosure.

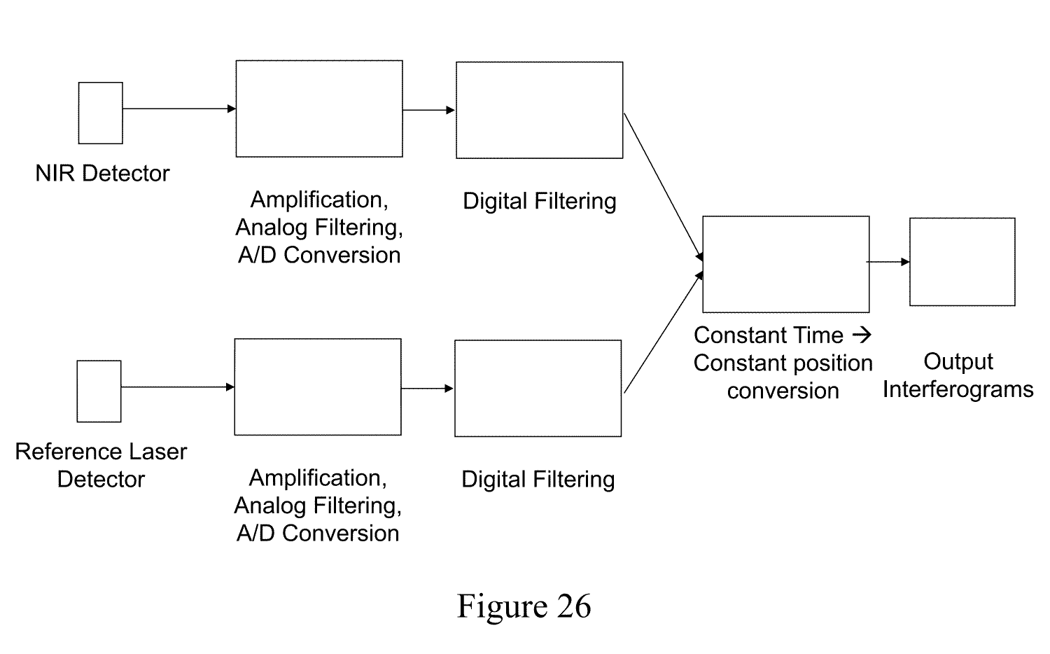

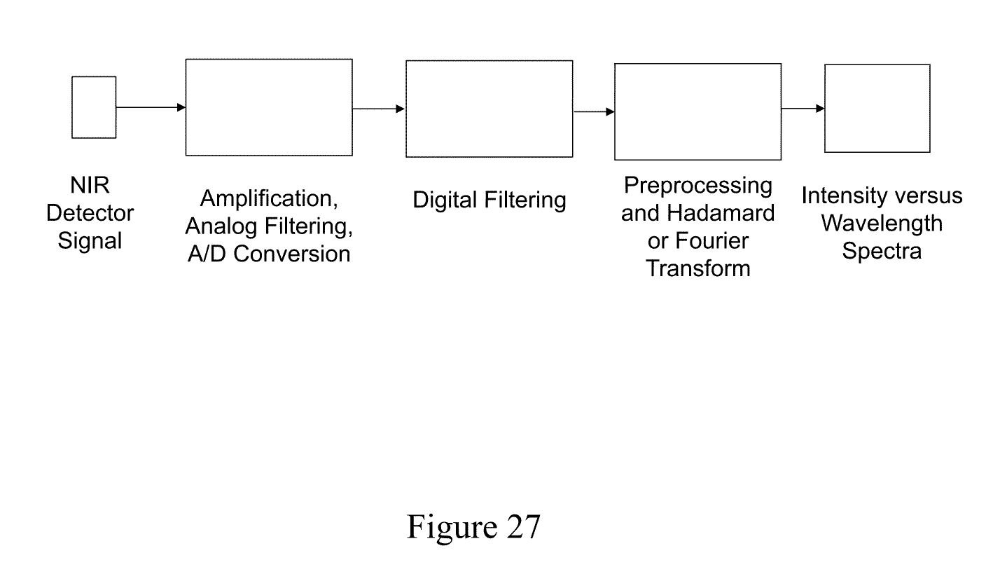

The data acquisition subsystem 400 converts the optical signal from the sampling subsystem 200 or spectrometer subsystem 300, depending on the systems orientation, into a digital representation. For example, FIG. 26 is a schematic representation of a data acquisition subsystem 400 that is applicable to the system diagram shown in FIG. 1. Alternatively, FIG. 27 shows a schematic representation of a data acquisition subsystem 400 that is applicable to the system diagram shown in FIG. 3. An important aspect of some embodiments of the present teachings that incorporate the combined illumination/modulation subsystem is that, similar to many interferometric spectrometers, only a single element detector is required to measure all desired wavelengths. This is advantageous as array detectors and their supporting electronics can be a significant drawback due to their expensive nature. One skilled in the art recognizes that different schematic representations of the data acquisition subsystem 400 are equally suitable for the system orientation shown in FIG. 3 and that the same or different data acquisition subsystem designs can be applicable to the orientations shown in FIGS. 1-2.

Regardless of the orientation of the noninvasive analyte measurement system, the purpose of the optical detector is to convert incident light into an electrical signal. Examples of detectors that are sensitive in the spectral range of 0.7 to 2.5 μm include InGaAs, InAs, InSb, Ge, PbS, Si, PtSi, and PbSe. An example embodiment of the present teachings can utilize a 1-mm, thermo-electrically cooled, extended range InGaAs detector that is sensitive to light in the 0.8 to 2.5 μm range. The extended range InGaAs detector has low Johnson noise and, as a result, allows Shot noise limited performance for the photon flux emanating from the sampling subsystem 200 or spectrometer subsystem 300. The extended InGaAs detector has peak sensitivity in the 2.0 to 2.5 μm spectral region. In comparison with the liquid nitrogen cooled InSb detector, InGaAs detectors can be more practical for a commercial product that uses these wavelengths. Also, InGaAs detectors can exhibit over 120 dbc of linearity in the 0.7 to 2.5 μm spectral region. Alternative detectors can be suitable if the measurement system utilizes alternative wavelength regions. For example, a silicon detector can be suitable if the wavelength range of interest were within the 300-1100 nm range.

Any photodetector can be used with the present teachings as long as the given photodetector satisfies basic sensitivity, noise, and speed requirements. The shunt resistance of the photodetector defines the Johnson or thermal noise of the detector. The Johnson noise of the detector should be low relative to the photon flux at the detector to ensure Shot noise limited performance. The terminal capacitance governs the cut-off frequency of the photodetector and may also be a factor in the high frequency noise gain of the photodetector amplifier. The photosensitivity is an important factor in the conversion of light to an electrical current and directly impacts the signal portion of the SNR equation.

The remainder of the data acquisition subsystem 400 amplifies and filters the electrical signal from the detector and then converts the resulting analog electrical signal to its digital representation with an analog to digital converter. The digital signal can then be filtered, re-sampling, or otherwise processed depending on the requirements of the specific embodiment under consideration. The analog electronics and ADC must support the high SNR and linearity inherent in the signal. To preserve the SNR and linearity of the signal, the data acquisition subsystem 400 can support at least 100 dbc of SNR plus distortion. The data acquisition subsystem 400 can produce a digitized representation of the signal. In some embodiments, a 24-bit delta-sigma ADC is used that can be operated at 96 or 192 kilohertz sampling rate. If system performance requirements permit, alternate analog to digital converters can be used in which the sample acquisition is synchronized with the light source modulation rather than captured at equal time intervals. The digitized signal can be passed to a computing subsystem 500 for further processing, as discussed below.

Further, the data acquisition subsystem 400 can utilize a constant time sampling, dual channel, delta-sigma analog-to-digital converter (ADC) to support the SNR and photometric accuracy requirements of the present non-invasive analyte measurement. In some embodiments, the delta-sigma ADC utilized supports sampling rates of over 100 kHz per channel, has a dynamic range in excess of 117 dbc and has total harmonic distortion less than −105 dbc. In a system that has only one channel of signal to digitize (instead of the two more common in delta-sigma ADC's), the signal can be passed into both inputs of the ADC and averaged following digitization. This operation can help to reduce any uncorrelated noise introduced by the ADC.

The constant time sampling data acquisition subsystem 400 has several distinct advantages over other methods of digitizing signals. These advantages include greater dynamic range, lower noise, reduced spectral artifacts; detector noise limited operation and simpler and less expensive analog electronics. In addition, the constant time sampling technique allows digital compensation for frequency response distortions introduced by the analog electronics prior to the ADC. This includes non-linear phase error in amplification and filtering circuits as well as the non-ideal frequency response of the optical detector. The uniformly sampled digital signal allows for the application of one or more digital filters whose cumulative frequency response is the inverse of the analog electronics' transfer function (see, e.g., U.S. Pat. No. 7,446,878, incorporated herein by reference).

The computing subsystem 500 performs multiple functions such as converting the digitized data obtained from the data acquisition subsystem 400 to spectra, performing spectral outlier checks on the spectra, spectral preprocessing in preparation for determination of the attribute of interest, determination of the attribute of interest, system status checks, all display and processing requirements associated with the user interface, and data transfer and storage, and internal and external communication (e.g. wireless, RS232, I2S, I2C, CAN, Ethernet, cell, satellite, USB, or other forms of communication). In some embodiments, the computing subsystem 500 is contained in a dedicated personal computer or laptop computer that is connected to the other subsystems of the teachings. In other embodiments, the computing subsystem is a dedicated, embedded computer within the noninvasive measurement device.

After converting the digitized data from the data acquisition subsystem 400 to spectra, the computer system can check the spectra for outliers or bad scans. An outlier or bad scan is one that violates the hypothesized relationship between the measured signal and the properties of interest. Examples of outlier conditions include conditions where the calibrated instrument is operated outside of the specified operating ranges for ambient temperature, ambient humidity, vibration tolerance, component tolerance, power levels, etc. In addition, an outlier can occur if the composition or concentration of the sample is different than the composition or concentration range of the samples used to calibrate the noninvasive analyte measurement system. The calibration model will be discussed as part of the calibration subsystem 600 later in this disclosure. Any outliers or bad scans can be deleted and the remaining good spectra can be averaged together to produce an average spectrum. In some embodiments, the average spectrum or individual spectra can be converted to absorbance by taking the negative base 10 logarithm (log 10) of the spectrum. The absorbance spectrum can be scaled in order to renormalize the noise. In other embodiments, such as those employing Raman spectroscopy, alternative spectral processing can be implemented such as the determination of an intrinsic Raman spectrum based upon the measured Raman spectrum. One skilled in the art recognizes that a variety of spectral processing and preprocessing steps can be performed on spectra prior to their use in determining the analyte concentration or property of interest. Some examples of such steps include, but are not limited to, determining one or more derivatives of the spectrum, noise scaling, variance scaling, background correction, background correction, scatter correction, and orthogonal signal correction.

The spectrum can be used to determine the attribute or property of interest in conjunction with a calibration model that is obtained from the calibration subsystem 600. After determination of the attribute of interest, the computing subsystem 500 can report the result 830, e.g., to the subject, to an operator or administrator, to a recording system, or to a remote location. The computing subsystem 500 can also report the level of confidence in the goodness of the result. If the confidence level is low, the computing subsystem 500 can withhold the result and ask for the measurement to be repeated. If required, additional information can be conveyed that directs the user to perform a corrective action. See, e.g., US Patent Application 2004/0204868, incorporated herein by reference. The results can be reported visually on a display, by audio and/or by printed means. Additionally, the results can be stored to form a historical record of the attribute. In other embodiments, the results can be stored and transferred to a remote monitoring or storage facility via wireless, cellular, internet, phone line, satellite, Ethernet, USB, blue tooth, I2S, I2C, CAN, RS232, cell phone service, or any other form of communication or communication protocol.

The computing subsystem 500 can include a central processing unit (CPU), memory, storage, a display and preferably a communication link. An example of a CPU is the Intel Pentium microprocessor. The memory can be, e.g., static random access memory (RAM) and/or dynamic random access memory. The storage can be accomplished with non-volatile RAM or a disk drive. A liquid crystal display can be suitable. The communication link can be, as examples, a high-speed serial link, wireless, cellular, internet, phone line, satellite, Ethernet, USB, blue tooth, I2S, I2C, CAN, RS232, cell phone service, or any other form of communication or communication protocol. The computer subsystem can, for example, produce attribute measurements from the received and processed spectra, perform calibration maintenance, perform calibration transfer, run instrument diagnostics, store a history of measured water concentrations and other pertinent information, and in some embodiments, communicate with remote hosts to send and receive data and new software updates.

The computing system 500 can also contain a communication link that allows transfer of a subject's measurement records and the corresponding spectra to an external database. In addition, the communication link can be used to download new software to the computer and update the multivariate calibration model. The computer system can be viewed as an information appliance. Examples of information appliances include personal digital assistants, web-enabled cellular phones and handheld computers.

The relationship between a spectrum and the property of interest (hydration state, TBW, and/or water concentration) may not be apparent upon visual inspection of the spectral data. Because this is the case, it is usually necessary that a multivariate mathematical relationship, or ‘model’, be constructed to determine the property of interest from spectra. The construction of such a model generally occurs in two phases: (i) collection of ‘calibration’ or ‘training’ data, and (ii) establishing a mathematical relationship between the training data and the attribute or reference concentrations represented in the training data.

In the case of measuring hydration, TBW, or water concentration in humans, during the collection of training data it can be desirable to collect spectra from many individuals that span a range of demographic conditions. Furthermore, these data should be collected over a variety of environmental conditions consistent with those expected in future use as well as over a range of concentrations for the property of interest (e.g. water concentration). It can be important to collect these data in a manner that minimizes the correlation between property of interest and other parameters that can result in spectral variation. The multivariate calibration model can empirically relate known values for the property of interest (e.g. concentrations) in a set of calibration samples to the measured spectra obtained from the calibration samples. This relationship can then be applied to subsequent measurements.

Partial Least Squares (PLS) regression is a well-established multivariate analysis method that has been applied to quantitative analysis of spectroscopic measurements and will be used for demonstrative purposes for the remainder of the disclosure. However, other multivariate analysis methods such as Principal Components Regression (PCR), Ridge Regression, Multiple Linear Regression (MLR) and Neural Networks are equally suitable for the present teachings. One skilled in the art will recognize that other methods of similar functionality are also applicable.

In PLS regression, a set of spectroscopic calibration measurements is acquired where each has a corresponding reference value for the property of interest (e.g. water concentration). The calibration spectral data are then decomposed into a series of factors (spectral shapes that are sometimes called loading vectors or latent variables) and scores (the magnitude of the projection of each spectrum onto a given factor) such that the squared covariance between the reference values and the scores on each successive PLS loading vector is maximized. The scores of the calibration spectra are then regressed onto the reference values in a multiple linear regression (MLR) step in order to calculate a set of spectral weights (one weight per wavenumber in the spectra) that minimizes the analyte measurement error of the calibration measurements in a least-squares sense. These spectral weights are called the regression vector of the calibration model. Once the calibration model is established, subsequent measurements are obtained by calculating the vector dot product of the regression vector and each measured spectrum.

The primary advantage of PLS and similar methods (commonly referred to as indirect methods) is that complete characterization of the sample and acquired spectra is not required (e.g. concentrations and identities of other constituents within the samples do not need to be known). Furthermore, inverse methods tend to be more robust at dealing with nonlinearities in the spectral measurement such as those caused by instrumental drift, light scattering, environmental noise, and chemical interactions.

Functionally, the goal of the multivariate calibration (PLS or otherwise) in the present teachings is to determine the part of the spectroscopic signal of the property of interest that is effectively orthogonal (contravariant) to the spectra of all interferents in the sample. This part of the signal is referred to as the net attribute signal (FIG. 4) and can be calculated using the regression vector (b) described above using equation 4. If there are no interfering species, the net attribute spectrum is equal to the pure spectrum of the property of interest. If interfering species with similar spectra to the attribute are present, the net attribute signal (NAS) will be reduced relative to the entire spectrum.

$$a . NAS = \frac{\hat{b}}{{‖ \hat{b} ‖}_{2}^{2}} \qquad Eq 4$$

Alternative calibration strategies can be used in place of, or in conjunction with, the above-described methods. For example, in some embodiments biometric enrollment information is acquired from each person that is measured on a device or network of devices. In such cases, the enrollment measurements can also be used to improve the accuracy and precision of the property of interest measurement. In this scenario, the calibration spectra can be mean-centered by subject (all spectra from a subject are located, the mean of those spectra is subtracted from each, and the “mean centered” spectra are returned to the spectral set). In this manner, the majority of inter-subject spectral differences caused by variations in physiology are removed from the calibration measurements and the range of spectral interferents correspondingly reduced. The centered spectra and associated analyte reference values are then presented to a multivariate analysis method such as partial least squares regression. This process is referred to as generating an “enrolled”, “generic”, or “tailored” calibration. Additional details on this approach is described in U.S. Pat. No. 6,157,041, entitled “Methods and Apparatus for Tailoring Spectroscopic Calibration Models,” incorporated by reference.

In practice, once a future, post calibration, subject is enrolled on a noninvasive device their enrollment spectrum can be subtracted from subsequent measurements prior to determining the property of interest using the generic calibration model. Similar to the mean-centering by subject operation of the calibration spectra, the subtraction of the enrollment spectrum removes the average spectroscopic signature of the subject while preserving the signal of the property of interest. In some embodiments, significant performance advantages can be realized relative to the use of a non-generic calibration method.

In other embodiments, the model for the performance athlete performs ongoing measurements to track multiple analytes through the curves represented at different points of TBW. These data points can further monitor trends and enhance recovery from dehydration.

In other embodiments, a hybrid calibration model can be used to determine the property of interest from spectra. In this case, the term hybrid model denotes that a multivariate calibration model was developed using a combination of in vitro and in vivo spectral data.

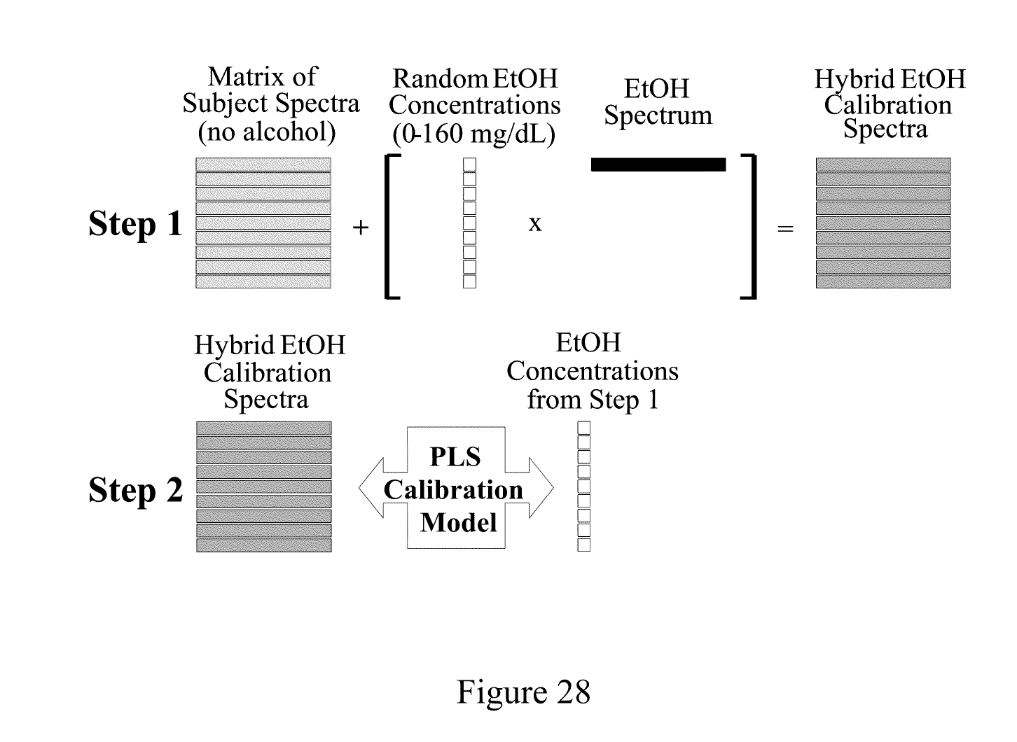

Light propagation through human tissue is a complex function of the sampling subsystem 200 design, physiological variables, the optical properties of the human tissue, and wavenumber. Consequently, the pathlength of light through human tissue has a wavenumber dependence that is not encountered in scatter-free transmission measurements. In order to account for the wavenumber dependence, the interaction between the sampling subsystem 200 and the scattering properties of human tissue can be modeled via Monte-Carlo simulation using a commercial optical ray-tracing software package (TracePro). The resulting model of the photon-tissue interactions can be used to generate an estimate of the effective pathlength of light through the tissue as a function of wavenumber. The effective pathlength (leff) is defined as:

$$a . {l}_{eff} ( v ) = \frac{{\sum }_{i = 1}^{N} {l}_{i} exp ( - {μ}_{a} ( v ) {l}_{i} )}{{\sum }_{i = 1}^{N} {l}_{i}} ,$$

Where v is wavenumber, li is the pathlength traversed by the ith ray in the Monte Carlo simulation [mm], N is the total number of rays in the simulation, and μₐ is the (wavenumber-dependent) absorption coefficient [mm⁻¹]. Due to its large absorption in vivo, water is the dominant analyte that affects the effective pathlength. Therefore, for the purposes of the effective pathlength calculation, the absorption coefficients used were those of water at physiological concentrations. For a hybrid model used to measure water concentration, the water absorbance spectrum (as measured in transmission) is scaled by the computed path function to form a corrected water spectrum representative of the wavenumber dependent pathlength measured by the diffuse reflectance optical sampler. This corrected spectrum forms the base spectrum for the mathematical addition of water variation to the calibration spectra. The in vivo data comprised noninvasive tissue spectra collected from multiple persons over multiple visits to a clinical facility, typically with no induced variations in the property of interest. A hybrid model is formed by adding the pathlength modified water pure component spectrum, weighted by various water “concentrations”, to the acquired in vivo data. The PLS calibration model was built by regressing the synthetic water concentrations on the hybrid spectral data. FIG. 28 is a schematic representation of a hybrid calibration formation process.

The use of hybrid calibration models, rather than calibration models built from spectra acquired from subjects who exhibited natural analyte variation, can provide significant advantages. The hybrid modeling process makes it possible to generate calibration spectra that contain larger variation in water concentration than would occur naturally. This can result in a stronger calibration with a wider range of analyte concentrations. This can be important because samples and people outside of a controlled clinical setting can exhibit water concentrations and/or hydration states outside the limits of safety in a clinical research setting. The hybrid calibration process also allows the prevention of correlations between the analyte of interest and the spectral interferents in tissue. Thus, the hybrid approach prevents the possibility that the measurement could spuriously track changes in other analytes in the body instead of water concentration.

Once formed, a calibration (generic or otherwise) should remain stable and produce accurate property of interest determinations over a desired period of time. This process is referred to as calibration maintenance and can comprise multiple methods that can be used individually or in conjunction. One method is to create the calibration in a manner that inherently makes it robust. Several different types of instrumental and environmental variation can affect the measurement capability of a calibration model. It is possible and desirable to reduce the magnitude of the effect of instrumental and environmental variation by incorporating this variation into the calibration model.

It is difficult, however, to span the entire possible range of instrument states during the calibration period. System perturbations can result in the instrument being operated outside the space of the calibration model. Examples of potentially problematic instrument and environmental variation include, but are not limited to, changes in the levels of environmental interferents such as water vapor or CO2 gas, changes in the alignment of the instrument's optical components, fluctuations in the output power of the instrument's illumination system, and changes in the spatial and angular distribution of the light output by the instrument's illumination system. Measurements made while the instrument is in an inadequately modeled state can exhibit measurement errors. In the case of in vivo optical measurements of analyte properties, these types of errors can result in erroneous measurements that degrade the utility of the system. Therefore it is often advantageous to use additional calibration maintenance techniques during the life of the instrument in order to continually verify and correct for the instrument's status.

Calibration maintenance techniques are discussed in U.S. patent application Ser. No. 09/832,608, “Optically Similar Reference Samples and Related Methods for Multivariate Calibration Models Used in Optical Spectroscopy,” and U.S. patent application Ser. No. 10/281,576, “Optically Similar Reference Samples,” and U.S. patent application Ser. No. 10/733,195, “Adaptive Compensation for Measurement Distortions in Spectroscopy,” each of which is incorporated herein by reference. These methods use an environmentally inert non-tissue sample, such as an integrating sphere, that optionally contains the property of interest, in order to monitor the instrument over time. The sample can be incorporated into the optical path of the instrument or interface with the sampling subsystem 200 in a manner similar to that of sample measurements. The sample can be used in transmission or in reflectance and can contain stable spectral features or contribute no spectral features of its own. The material can be a solid, liquid, or gel material as long as its spectrum is stable or predictable over time. Any unexplained change in the spectra acquired from the sample over time indicate that the instrument has undergone a perturbation or drift due to environmental effects. The spectral change can then be used to correct subsequent sample measurements from humans in order to ensure and accurate attribute measurement.

Another means for achieving successful calibration maintenance is to update the calibration using measurements acquired on the instrument over time. Usually, knowledge of the reference value of the analyte property of interest is required in order to perform such an update. However, in some applications, it is known that the reference value is usually, but not always, a specific value. In this case, this knowledge can be used to update the calibration even though the specific value of the analyte property is not known for each measurement. Thus, the calibration can be updated to include new information as it is acquired in the field. This approach can also be used to perform calibration transfer as measurements with presumed values can be used at the time of system manufacture or installation in order to remove any system-specific bias in the analyte property measurements of interest. The calibration maintenance update or calibration transfer implementation can be accomplished by a variety of means such as, but not limited to, orthogonal signal correction (OSV), orthogonal modeling techniques, neural networks, inverse regression methods (PLS, PCR, MLR), direct regression methods (CLS), classification schemes, simple median or moving windows, principal components analysis, or combinations thereof.

Once a calibration is formed, it is desirable to transfer the calibration to existing and future instruments. This process is commonly referred to as calibration transfer. While not required, calibration transfer prevents the need for a calibration to be built on each system that is manufactured. This represents a significant time and cost savings that could result in the difference between success and failure of a commercial product. Calibration transfer arises from the fact that optical and electronic components vary from unit to unit which, in aggregate, results in differences in the spectra obtained from multiple instruments. For example, the responsivity of two detectors can also differ significantly, which can result in spectral differences between instruments.

Similar to calibration maintenance, multiple methods can be used in order to effectively achieve calibration transfer. The first method is to build the calibration with multiple instruments. The presence of multiple instruments allows the spectral variation associated with instrument differences to be determined and made effectively orthogonal to the attribute signal during the calibration formation process. While this does approach reduces the net attribute signal, it can be an effective means of calibration transfer.

Additional calibration transfer methods involve explicitly determining the difference in the spectral signature of a system relative to those used to build the calibration. In this case, the spectral difference can then be used to correct a spectral measurement prior to attribute prediction on a system or it can be used to correct the predicted attribute value directly. The spectral signature specific to an instrument can be determined from the relative difference in spectra of a stable sample acquired from the system of interest and those used to build the calibration. Many suitable approaches and algorithms for effective calibration transfer are known in the art; some of which are summarized in “Standardization and Calibration Transfer for Near Infrared Instruments: a Review”, by Tom Fearn in the Journal of Near Infrared Spectroscopy, vol. 8, pp. 229-244 (2001). Note that these approaches and algorithms are equally suited to other spectroscopic techniques such as Raman measurements. The samples described in the calibration maintenance section can also be applicable to calibration transfer. See, e.g. U.S. Pat. No. 6,441,388, incorporated herein by reference.

Depending on the application of interest, the measurement of an analyte property can be considered in terms of two modalities. The first modality is “walk up” or “universal” and represents an analyte property determination wherein prior measurements of the sample (e.g. subject) are not used in determining the analyte property from the current measurement of interest. Thus, no prior knowledge of that person is available for use in the current determination of the analyte property.

The second modality is termed “enrolled” or “tailored” and represents situations where prior measurements from the sample or subject are available for use in determining the analyte property of the current measurement. Additional information regarding embodiments of enrolled and tailored applications can be found in U.S. Pat. Nos. 6,157,041 and 6,528,809, each of which is incorporated herein by reference. In enrolled applications, the combination of the analyte property measurement with a biometric measurement can be particularly advantageous.

Biometric identification describes the process of using one or more physical or behavioral features to identify a person or other biological entity. There are two common biometric modes: identification and verification. Biometric identification attempts to answer the question of, “do I know you?” The biometric measurement device collects a set of biometric data from a target individual. From this information alone it assesses whether the person was previously enrolled in the biometric system. Systems that perform the biometric identification task, such as the FBI's Automatic Fingerprint Identification System (AFIS), are generally very expensive (several million dollars or more) and require many minutes to detect a match between an unknown sample and a large database containing hundreds of thousands or millions of entries. In biometric verification the relevant question is, “are you who you say you are?” This mode is used in cases where an individual makes a claim of identity using a code, magnetic card, or other means, and the device uses the biometric data to confirm the identity of the person by comparing the target biometric data with the enrolled data that corresponds with the purported identity. The present apparatus and methods for monitoring the presence or concentration of water or hydration state can use either biometric mode.

There also exists at least one variant between these two modes that is also suitable for use in the present teachings. This variant occurs in the case where a small number of individuals are contained in the enrolled database and the biometric application requires the determination of only whether a target individual is among the enrolled set. In this case, the exact identity of the individual is not required and thus the task is somewhat different (and often easier) than the identification task described above. This variant might be useful in applications where the biometric system is used in methods where the tested individual must be both part of the authorized group and sober but their specific identity is not required. The term “identity characteristic” includes all of the above modes, variants, and combinations or variations thereof.

There are three major data elements associated with a biometric measurement: calibration, enrollment, and target spectral data. The calibration data are used to establish spectral features that are important for biometric determinations. This set of data consists of series of spectroscopic tissue measurements that are collected from an individual or individuals of known identity. Preferably, these data are collected over a period of time and a set of conditions such that multiple spectra are collected on each individual while they span nearly the full range of physiological states that a person is expected to go through. In addition, the instrument or instruments used for spectral collection generally should also span the full range of instrumental and environmental effects that it or sister instruments are likely to see in actual use. These calibration data are then analyzed in such a way as to establish spectral wavelengths or “factors” (i.e. linear combinations of wavelengths or spectral shapes) that are sensitive to between-person spectral differences while minimizing sensitivity to within-person, instrumental (both within- and between-instruments), and environmental effects. These wavelengths or factors are then used subsequently to perform the biometric determination tasks.

The second major set of spectral data used for biometric determinations is the enrollment spectral data. The purpose of the enrollment spectra for a given subject or individual is to generate a “representation” of that subject's unique spectroscopic characteristics. Enrollment spectra are collected from individuals who are authorized or otherwise required to be recognized by the biometric system. Each enrollment spectrum can be collected over a period of seconds or minutes. Two or more enrollment measurements can be collected from the individual to ensure similarity between the measurements and rule out one or more measurements if artifacts are detected. If one or more measurements are discarded, additional enrollment spectra can be collected. The enrollment measurements for a given subject can be averaged together, otherwise combined, or stored separately. In any case, the data are stored in an enrollment database. In some cases, each set of enrollment data are linked with an identifier (e.g. a password or key code) for the persons on whom the spectra were measured. In the case of an identification task, the identifier can be used for record keeping purposes of who accessed the biometric system at which times. For a verification task, the identifier is used to extract the proper set of enrollment data against which verification is performed.

The third and final major set of data used for the biometric system is the spectral data collected when a person attempts to use the biometric system for identification or verification. These data are referred to as target spectra. They are compared to the measurements stored in the enrollment database (or subset of the database in the case of identity verification) using the classification wavelengths or factors obtained from the calibration set. In the case of biometric identification, the system compares the target spectrum to all of the enrollment spectra and reports a match if one or more of the enrolled individual's data is sufficiently similar to the target spectrum. If more than one enrolled individual matches the target, then either all of the matching individuals can be reported, or the best match can be reported as the identified person. In the case of biometric verification, the target spectrum is accompanied by an asserted identity that is collected using a magnetic card, a typed user name or identifier, a transponder, a signal from another biometric system, or other means. The asserted identity is then used to retrieve the corresponding set of spectral data from the enrollment database, against which the biometric similarity determination is made and the identity verified or denied. If the similarity is inadequate, then the biometric determination is cancelled and a new target measurement may be attempted.

In one method of verification, principle component analysis is applied to the calibration data to generate spectral factors. These factors are then applied to the spectral difference taken between a target spectrum and an enrollment spectrum to generate Mahalanobis distance and spectral residual magnitude values as similarity metrics. Identify is verified only if the aforementioned distance and magnitude are less than a predetermined threshold set for each. Similarly, in an example method for biometric identification, the Mahalanobis distance and spectral residual magnitude are calculated for the target spectrum relative each of the database spectra. The identity of the person providing the test spectrum is established as the person or persons associated with the database measurement that gave the smallest Mahalanobis distance and spectral residual magnitude that is less than a predetermined threshold set for each.

In an example method, the identification or verification task is implemented when a person seeks to perform an operation for which there are a limited number of people authorized (e.g., perform a spectroscopic measurement, enter a controlled facility, pass through an immigration checkpoint, etc.). The person's spectral data is used for identification or verification of the person's identity. In this preferred method, the person initially enrolls in the system by collecting one or more representative tissue spectra. If two or more spectra are collected during the enrollment, then these spectra can be checked for consistency and recorded only if they are sufficiently similar, limiting the possibility of a sample artifact corrupting the enrollment data. For a verification implementation, an identifier such as a PIN code, magnetic card number, username, badge, voice pattern, other biometric, or some other identifier can also be collected and associated with the confirmed enrollment spectrum or spectra.

In subsequent use, biometric identification can take place by collecting a spectrum from a person attempting to gain authorization. This spectrum can then be compared to the spectra in the enrolled authorization database and an identification made if the match to an authorized database entry was better than a predetermined threshold. The verification task is similar, but can require that the person present the identifier in addition to a collected spectrum. The identifier can then be used to select a particular enrollment database spectrum and authorization can be granted if the current spectrum is sufficiently similar to the selected enrollment spectrum. If the biometric task is associated with an operation for which only a single person is authorized, then the verification task and identification task are the same and both simplify to an assurance that the sole authorized individual is attempting the operation without the need for a separate identifier.

The biometric measurement, regardless of mode, can be performed in a variety of ways including linear discriminant analysis, quadratic discriminant analysis, K-nearest neighbors, neural networks, and other multivariate analysis techniques or classification techniques. Some of these methods rely upon establishing the underlying spectral shapes (factors, loading vectors, eigenvectors, latent variables, etc.) in the intra-person calibration database, and then using standard outlier methodologies (spectral F ratios, Mahalanobis distances, Euclidean distances, etc.) to determine the consistency of an incoming measurement with the enrollment database. The underlying spectral shapes can be generated by multiple means as disclosed herein.

First, the underlying spectral shapes can be generated based upon simple spectral decompositions (eigen analysis, Fourier analysis, etc.) of the calibration data. The second method of generating underlying spectral shapes relates to the development of a generic model as described in U.S. Pat. No. 6,157,041, entitled “Methods and Apparatus for Tailoring Spectroscopic Calibration Models,” which is incorporated by reference. In this application, the underlying spectral shapes are generated through a calibration procedure performed on intra-person spectral features. The underlying spectral shapes can be generated by the development of a calibration based upon simulated constituent variation. The simulated constituent variation can model the variation introduced by real physiological or environmental or instrumental variation or can be simply be an artificial spectroscopic variation. It is recognized that other means of determining underlying shapes would be applicable to the identification and verification methods of the present teachings. These methods can be used either in conjunction with, or in lieu of the aforementioned techniques.

In addition to disposables to ensure subject safety, disposable calibration check samples can be used to verify that the instrument is in proper working condition. In many commercial applications of analyte measurements, the status of the instrument must be verified to ensure that subsequent measurements will provide accurate concentrations or attribute estimates. The instrument status is often checked immediately prior to a subject measurement. In some embodiments, the calibration check sample can include the analyte of interest. In other embodiments, the check sample can be an environmentally stable and spectrally inert sample, such as an integrating sphere. The check sample can be a gas or liquid that is injected or flowed through a spectroscopic sampling chamber. The check sample can also be a solid, such as a gel, that may contain the analyte of interest. The check sample can be constructed to interface with the sampling subsystem or it can be incorporated into another area of the optical path of the system. These examples are meant to be illustrative and are not limiting to the various possible calibration check samples.

The present teachings also comprise methods for measurement of the direction and magnitude of concentration changes of tissue constituents, such as water, using spectroscopy. The non-invasive measurement obtained from the current teachings is inherently semi-time resolved. This allows attributes, such as water concentration, to be determined as a function of time. The time resolved water concentrations could then be used to determine the rate and direction of change of the water concentration. In addition, the direction of change information can be used to partially compensate for any difference in blood and non-invasive water concentration that is caused by physiological kinetics. See U.S. Pat. No. 7,016,713, and US Application 20060167349, each of which is incorporated herein by reference. A variety of techniques for enhancing the rate and direction signal have been uncovered. Some of these techniques include heating elements, rubrifractants, and index-matching media. They should not be interpreted as limiting the present teachings to these particular forms of enhancement or equilibration. These enhancements are not required to practice the present teachings, but are included for illustrative purposes only.

Another aspect of non-invasive analyte measurements is the safety of the subjects during the measurements. In order to prevent measurement contamination or transfer of pathogens between subjects it is desirable, but not necessary, to use disposable cleaning agents and/or protective surfaces in order to protect each subject and prevent fluid or pathogen transfer between subjects. For example, in some embodiments an isopropyl wipe can be used to clean each subject's sampling site and/or the sampling subsystem surface prior to measurement. In other embodiments, a disposable thin film of material such as ACLAR could be placed between the sampling subsystem and the subject prior to each measurement in order to prevent physical contact between the subject and the instrument. In other embodiments, both cleaning and a film could be used simultaneously. As mentioned in the sampling subsystem portion of this disclosure, the film can also be attached to a positioning device and then applied to the subject's sampling site. In this embodiment, the positioning device can interface with the sampling subsystem and prevent the subject from moving during the measurement while the film serves its protective role.

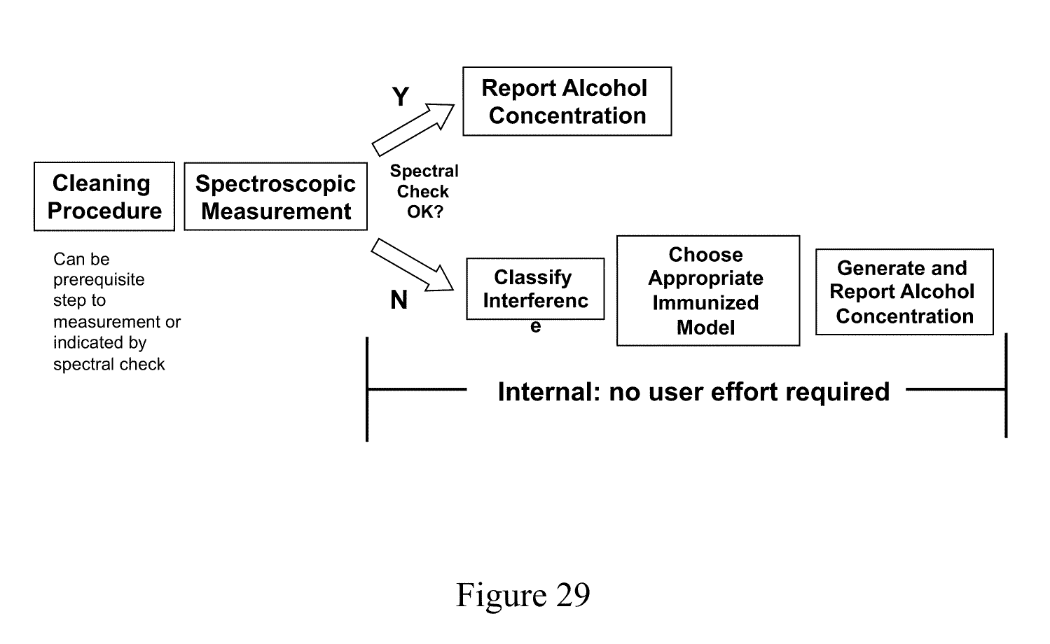

In subject measurements the presence of topical interferents on the sampling site is a significant concern. Many topical interferents have spectral signatures in the near infrared region and can therefore contribute significant measurement error when present. The present teachings deal with the potential for topical interferents in three ways that can be used individually or in conjunction. FIG. 29 shows a flow diagram that describes a method for combining the three topical interferent mitigation approaches into one combined process. First, a disposable cleaning agent similar to that described in the subject safety section can be used. The use of the cleaning agent can either be at the discretion of the system operator or a mandatory step in the measurement process. Multiple cleaning agents can also be used that individually target different types of topical interferents. For example, one cleaning agent can be used to remove grease and oils, while another could be used to remove consumer goods such as cologne or perfume. The purpose of the cleaning agents is to remove topical interferents prior to the attribute measurement in order to prevent them from influencing the accuracy of the system.

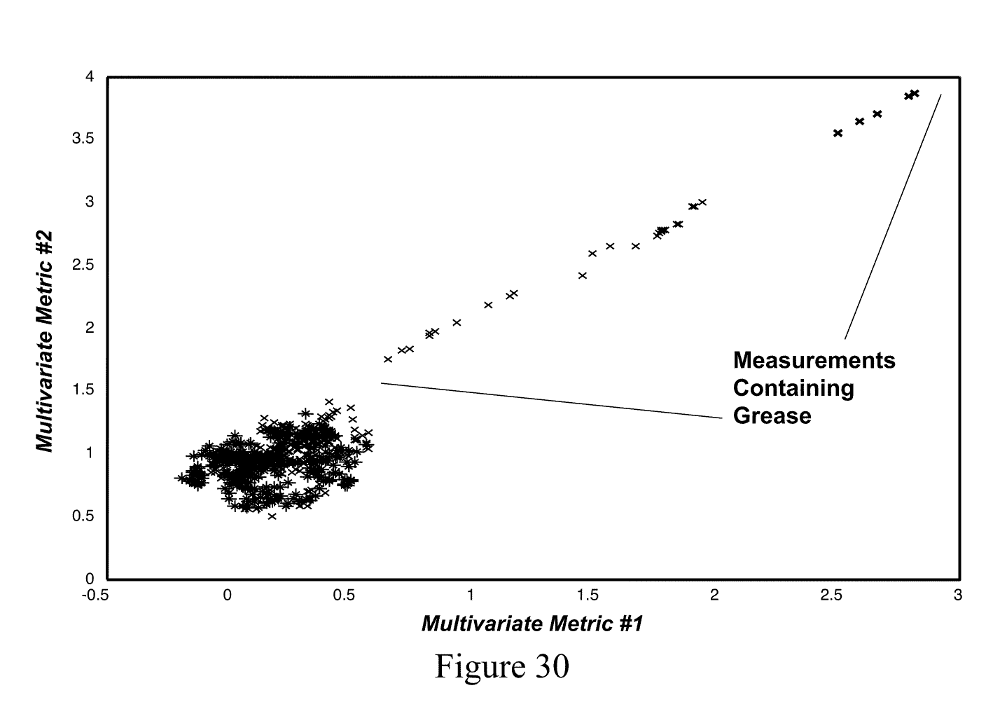

The second method for mitigating the presence of topical interferents is to determine if one or more interferents is present on the sampling site. The multivariate calibration models used in the calibration subsystem offer inherent outlier metrics that yield important information regarding the presence of un-modeled interferents (topical or otherwise). As a result, they provide insight into the trustworthiness of the attribute measurement. FIG. 30 shows example outlier metric values from noninvasive measurements using the present teachings acquired during the clinical studies. All of the large metric values (clearly separated from the majority of the points) correspond to measurements where grease had been intentionally applied to the subject's sampling site. These metrics do not specifically identify the cause of the outlier, but they do indicate that the associated attribute measurement is suspect. An inflated outlier metric value (a value beyond a fixed threshold, for example) can be used to trigger a fixed response such as a repeat of the measurement, application of an alternative calibration model, or a sampling site cleaning procedure. This is represented in FIG. 30 as the “Spectral Check OK” decision point.

The final topical interferent mitigation method involves adapting the calibration model to include the spectral signature of the topical interferent. The adapted calibration model can either be created on demand or selected from an existing library of calibration models. Each calibration in the library would be targeted at mitigating a different interferent or class of interferents such as oils. In some embodiments, the appropriate calibration model can be chosen based on the portion of an acquired spectrum that is unexplained by the original calibration model. This portion of the spectrum is referred to as the calibration model residual. Because each topical interferent or class of interferents has a unique near infrared spectrum, the calibration model residual can be used to identify the topical interferent.

The model residual or the pure spectrum (obtained from a stored library) of the interferents can then be incorporated into the spectra used to form the calibration. The multivariate calibration is then reformed with the new spectra such that the portion of the attribute signal that is orthogonal to the interferent can be determined. The new calibration model is then used to measure the attribute of interest and thereby reduce the effects of the topical interferent on attribute measurement accuracy. The resulting model will reduce the effect of the interferent on the analyte measurement at the expense of measurement precision when no interferents are present. This process is referred to as calibration immunization. The immunization process is similar to the hybrid calibration formation process shown in FIG. 28, but includes the additional step of the mathematical addition of the interferent's spectral variation. It should be noted that, due to the impact of the immunization process on measurement precision, it could be desirable to identify possible interferents for each measurement and immunize specifically against them rather than attempt to develop a calibration that is immunized against all possible interferents. Additional details can be found in US 20070142720, incorporated herein by reference.

It is important to note that the present teachings also envision several embodiments of analyte measurement systems incorporating broadband light sources rather than narrowband solid-state light sources. An example light source is a ceramic element such as those commonly used as igniters for furnaces and stoves. These light sources have a lower color temperature than standard filament lamps and are therefore more efficient in the near-infrared spectral region. These sources also have comparatively large emissive surfaces that are less sensitive to spatial effects that are encountered throughout the lifetime of the light source. An additional advantage of igniter-based light sources is a substantially longer lifetime when compared to filament lamps. In these embodiments, the broad blackbody source can be converted to multiple, narrow light sources using optical filters such as, but not limited to, linearly variable filters (LVF's), dielectric stacks, distributed Bragg gratings, photonic crystal lattice filters, polymer films, absorption filters, reflection filters, etalons, dispersive elements such as prisms and gratings, and quantum dot filters. The resulting multiple bands of wavelengths can be modulated by a Fourier scheme or Hadamard mask.

Some embodiments eliminate the drawbacks of filament-based light sources by replacing them with alternative sources of IR and NIR light. Ceramic-based blackbody light sources and semiconductor-based light sources offer several advantages including elimination of the glass envelope, higher efficiency (less light in unwanted spectral regions), and more stable spatial emission. Consequently, the ceramic and semiconductor light sources offer an improved foundation for subsequent spatial and angular homogenization. Furthermore, due to the improved optical efficiency, these light sources do not require undesired wavelengths to be optically filtered prior to sample illumination. The reduction of the illumination source as an instrument variance or interferent has been found to improve the ability to build an optical system and model that can accurately predict analyte concentrations in turbid media such as tissue. Some embodiments of the present teachings provide this illumination stability by collecting and modifying the output emitted by a light source prior to illuminating the sample under investigation.

Some embodiments relate to methods for minimizing spectroscopic variances due to radiation emitters of angular and/or spatial homogenization. Angular homogenization is any process that takes an arbitrary angular distribution, or intensity (W/sr), of emitted radiation, and creates a more uniform angular distribution. Spatial homogenization is the process of creating a more uniform distribution of irradiance (W/m2) across an output or exit face.

All practical light sources produce a non-uniform irradiance distribution due to their physical structure. Thus, radiation emitter differences (e.g., a different source) will result in different non-uniform irradiance distributions. These differences in irradiance distribution can translate into spectroscopic differences between light sources. Thus, an objective of the teachings is to take different irradiance distributions due to emitter differences and create similar or ideally the same irradiance distribution. A preferred method of creating similar irradiance distributions is to create a uniform irradiance distribution.

Differences in the radiation emitter will also result in differences in angular distribution. As above, an objective of this teaching is to create an illumination system where radiation emitter differences do not affect the angular distribution observed by the sample or at the input to the spectrometer. One mechanism is to create a uniform angular distribution. An ideal angular homogenizer would uniformly distribute the light over a sphere (4 pi sr) regardless of the angular distribution from the emitter. An ideal reflective angular homogenizer would uniformly distribute light over a hemisphere (2 pi sr). Due to the fact that other optical components in the system must collect light within a defined numerical aperture, ideal homogenizers are typically very inefficient. Thus, the instrument designer must weigh the benefits of angular homogenization with loss in optical efficiency. Regardless of the specific embodiment, angular homogenization is a critical component in the realization of an illumination system that has reduced sensitivity to emitter differences.

The present teachings provides a system for producing spatially and angularly homogenized light from an irregular emitter and using the homogenized light for spectral analysis. The resulting homogenized radiation illuminates the sample or sampler in a consistent and reproducible form, thus allowing for accurate and dependable spectroscopic measurements.

An additional benefit of the current teachings is spatial homogenization. The color temperature of filament and ceramic light sources is not spatially uniform across the entire emissive area of the source. Thus, color temperature variations across the filament will result in spectral differences across the filament length. These spectral differences due to color temperature variations or other filament differences can be different between emitters and can change over time. Thus, an additional objective of the teachings is to take the different spectral distribution due to spatial heterogeneity of the emitter and create a preferably uniform spectral distribution at all spatial locations at the output of the illumination system.

Benefits of the present teachings can be illustrated by the familiar occurrence of routine maintenance to a spectrometer. It is common for radiant light sources to burn out. Although application dependent, the replacement of the light source can result in analyte measurement errors and can necessitate recalibration of the spectrometer following the light source replacement. In systems intended for commercial use by unskilled operators, recalibration is not desired. With the present teachings, however, differences in the light source are irrelevant and proper performance of the optical measurement system is maintained. Regardless of the spatial and angular characteristics of the radiation emitted by the light source, the use of the illumination systems of the present teachings will result in radiation incident on the sample that remains substantially spatially and angularly homogenized. Thus, a light source change will not detract from the accuracy and dependability of molecular absorbance measurements using the present teachings.

The present teachings further provide a system for providing illumination to biological tissue samples. More specifically, the system is particularly suited for spectroscopic illumination of biological tissues for determining and quantifying the concentration of specific analytes within or other characteristics of the tissue. The present teachings enable a practitioner to construct and operate an illumination device that permits measurements with a high signal-to-noise ratio (SNR) while minimizing thermal damage to biological tissue. With a high SNR, chemometric models may be developed for differentiating between a particular analyte and interferents similar to that analyte. The present teachings allows for spectroscopic analysis of turbid media by satisfying the following conditions:

(1) The radiation emitted by the present teachings contains wavelengths useful for measuring the analyte of interest. The radiation may be continuous versus wavelength, in locally continuous bands, or selected to particular wavelengths. The result is radiation that encompasses the wavelength regions that contain the NIR or IR spectral “fingerprint” for the analyte of interest. For the noninvasive measurement of ethanol using NIR spectroscopy, this wavelength region spans approximately from 0.8 to 2.5 μm.

(2) The radiation emitted by the present teachings is of sufficiently high spectral radiance to provide a high signal-to-noise ratio in the spectral region of interest. In the measurement of ethanol using NIR spectroscopy, for example, the radiation from a ceramic light source or one or more semiconductor light sources concentrated with one or more optical elements, such as lenses and or mirrors, will provide a spectral radiance that satisfies this condition.

(3) The spectral radiance is generally invariant when subjected to changes in the spectral exitance of the emitter. Reasonably expected changes in the spectral exitance are those due to rotation and/or small translation of the emitter, or replacement of the emitter with another emitter of the same general construction.

By satisfying the above conditions, the ceramic-based light sources of the present teachings eliminate the need for recalibration due to illumination variability (light source changes, source aging, source rotation or movement) or, in some embodiments, development of a chemometric model that compensates for such changes. Simple maintenance such as replacing the light source would not necessitate recalibration or the development of chemometric models sensitive to light source changes. Furthermore, rotations and translations of the light source caused by jolts, bumps, and other similar vibrations would have minimal effects on the accuracy of a test.

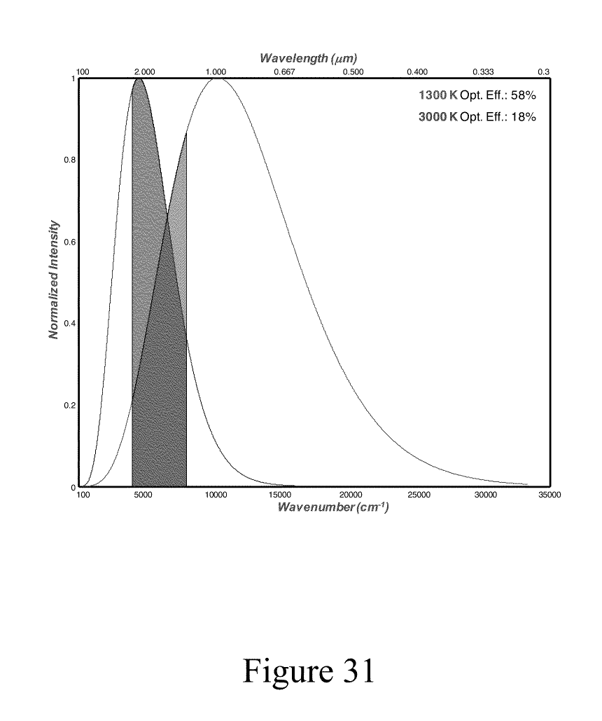

Most light sources used in spectroscopy are blackbody radiators. The light emitted by a blackbody radiator is governed by Plank's law which indicates that the intensity of the light emitted is a function of wavelength and the temperature of the blackbody. FIG. 31 shows normalized NIR spectra of 1300 and 3000 K blackbody radiators over the 100-33000 cm (100-0.3 μm) range with the 4000-8000 cm (2.5-1.25 μm) range used by the device shaded. 1300 K is a reasonable temperature for the ceramic-based blackbody light source the technology currently employs and 3000 K is a reasonable temperature for Quartz Tungsten Halogen (QTH) lamps that are often employed in spectroscopic applications. FIG. 31 indicates that the optical efficiency of both blackbody light sources is not ideal in that a significant amount of light is emitted at wavelengths outside the region of interest with the optical efficiency of the ceramic light source being 58% and the QTH only 18%.

In addition to optical efficiency, blackbody light sources can have poor electrical efficiency. Practical blackbody light sources require a significant amount of electrical power, not all of which is converted to emitted light. Electrical and optical power measurements on hundreds of ceramic blackbody light sources that show an average of 1.1 W of optical power at an average of 24 W of electrical power (4.4% electrical efficiency). When combined with the optical efficiency of 58%, the overall efficiency of the ceramic blackbody is approximately 2.5%. In other words, at 24 W of electrical power, approximately 0.6 W of optical power is emitted in the 4000 to 8000 cm region of interest. Further losses are incurred as not all light emitted by the source is collected by the remainder of the optical system.

As indicated by the low electrical efficiency, most of the applied electrical power is converted to heat that has a detrimental beyond the higher than desired power requirement. The heat generated by the blackbody light source can have an impact on the thermal state and stability of the spectroscopic measurement device. Consequently, in some situations the device must be powered on and allowed to reach thermal equilibrium prior to performing measurements. The equilibration time associated with the blackbody light source can range from minutes to hours that can be disadvantageous in some situations.

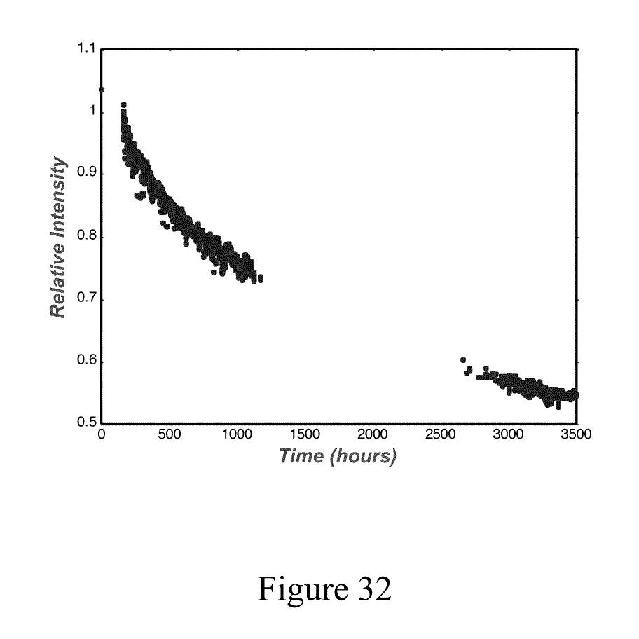

Blackbody light sources exhibit an aging effect as the material resistance changes. From an optical perspective, there are to significant implications associated with the light source aging. First, as the resistance increases the amount of optical power emitted decreases. FIG. 32 shows the measured intensity over time observed for a demonstrative ceramic blackbody light source that exhibits a 50% reduction in power over 3500 hours. The intensity degradation over time tends to be exponential in nature and can necessitate replacement of the light source at regular intervals that can be disadvantageous in some deployment environments. Second, the temperature of the light source changes which alters the distribution of the light as a function of wavelength. Depending on the severity of the color temperature change, the stability of the spectroscopic device over time can be impacted.

LEDs and other solid-state light sources, in contrast, are narrower in their emission profiles, which allow the ability to concentrate the emitted light in the region of interest. The range of available LED's allows the investigation of their combination to form a light source system that spans the region of interest while minimizing light output at lower and higher wavenumber that are not employed by the noninvasive measurement. Thus, the resulting system will exhibit an improved optical efficiency. It is important to note, that in contrast to other embodiments involving modulation schemes previously discussed, the objective of these embodiments of solid state light sources is to use multiple solid state light sources to collectively mimic the optical properties of a blackbody light source in a more efficient package.

In some embodiments, no single LED can viably replace a blackbody light source, as the spectral emission profiles do not span the entire region of interest. Consequently, multiple LED's could need to be optically combined in order to generate a suitable light source subsystem. The number of LED's that can be incorporated into the light source subsystem is ultimately determined by the area and angular acceptance of the optical system and the size and angular divergence of the individual LEDs. The determination of the optimal combination of LED's involves optical and mechanical design and spectroscopic analysis. In some embodiments, the magnitude of each LED's emission can be influenced by changing the input power to the respective LED, adding more LED's of that type, or a combination thereof. Furthermore, within a given spectral region of interest, some wavelengths could be more important than others to a given application such as hydration measurements in tissue. The narrow profiles exhibited by the LED's could allow better fine tuning of the relative intensities of the wavelengths as compared to blackbody light sources.

LED's do not critically fail in any manner similar to filament lamps. Instead they exhibit intensity degradation over time. As a result, the lifetimes of LED's are measured in terms of the time in hours required for the average LED of a given type to reach 50% of its original intensity (T50). The lifetimes of LED's, for example, range from 50,000 to 100,000 hours. As a result, LED's offer the potential for a 10× improvement in light source life and a corresponding reduction in the need for routine maintenance relative to blackbody light sources.

LEDs and semiconductor lasers such as VCSEL's can have small emissive areas when compared to their blackbody counterparts that is driven by the size of the semiconductor die itself. The photon emission cannot occur outside of the area of the die as it is generated within the semiconductor structure. The small size (a common emissive area is a 0.3 mm×0.3 mm square or 0.09 mm2) can be advantageous in that any heterogeneity within that area will be insignificant relative to size of the output of the illumination system (which can be several mm2 or larger depending on the application). Thus, as long as the die (or dies if multiple semiconductors are employed) does not physically move, the spatial output will be very stable. The objective of subsequent spatial homogenizers is then to uniformly distribute the light emitted by the die across the entire area of the illumination system output.

Another advantage of semiconductor light sources such as LED's is the ability to incorporate more than one die into the same physical package. As the output of an LED is typically spectrally narrower than a blackbody light source, multiple LED's of different types (e.g. peak wavelength of emission) can be combined to increase the spectral range of the illumination system. Furthermore, additional LED's of the same type can be included in order to increase the optical power at the corresponding wavelengths. Such approaches allow an unprecedented level of control over both the specific wavelengths and relative intensities emitted by an illumination system. This could be used to accentuate wavelengths important to a given analyte of interest such as water, while reducing the output at less-important wavelengths. Whether the set of LED's is all of the same type or a mixture, up to several hundred LED's could be incorporated into the same package while retaining an integrated optical area consistent with use in noninvasive analyte measurements such as water.

Another advantage of semiconductor light sources is the ability to select which light sources is on at a given time as well as tune their output via voltage or current and temperature. Consequently, a single illumination system could be optimized for measurements of multiple analytes. For example, when measuring water in tissue a given set of LEDs could be activated. Likewise, a different set could be activated when measuring a different analyte such as cholesterol or glucose.

The peak emission wavelength of solid-state light sources, particularly lasers, can be influenced by changing the thermal state or electrical properties (e.g. drive current or voltage) of the light source. In the case of semi conductor lasers, changing the thermal state and electrical properties alters the optical properties or physical dimensions of the lattice structure of the semiconductor. The result is a change in the cavity spacing within the device, which alters the peak wavelength emitted. Since solid-state light sources exhibit these effects, when they are used in spectroscopic measurement systems the stability of the peak wavelength of emission and its associated intensity can be important parameters.

Consequently, during a measurement control of both the thermal state and electrical properties of each light source can be advantageous in terms of overall system robustness and performance. Furthermore, the change in optical properties caused by thermal state and electrical conditions can be leveraged to allow a single light source to be tuned to multiple peak wavelength locations. This can result in analyte property measurement systems that can measure more wavelength locations than the number of discrete light sources that can reduce system cost and complexity.

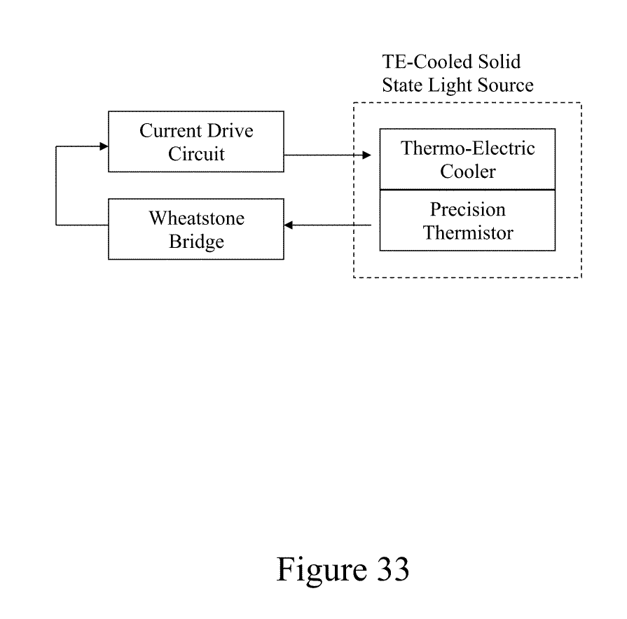

Temperature stabilization can be achieved using multiple approaches. In some embodiments, a light source or light sources can be stabilized by raising the temperature above (or cooling below) ambient conditions with no additional control of the temperature. In other embodiments, the light source or light sources can be actively controlled to a set temperature (either cooled or heated) using a control loop. A diagram of a temperature control loop circuit suitable for the present teachings is shown in FIG. 33.

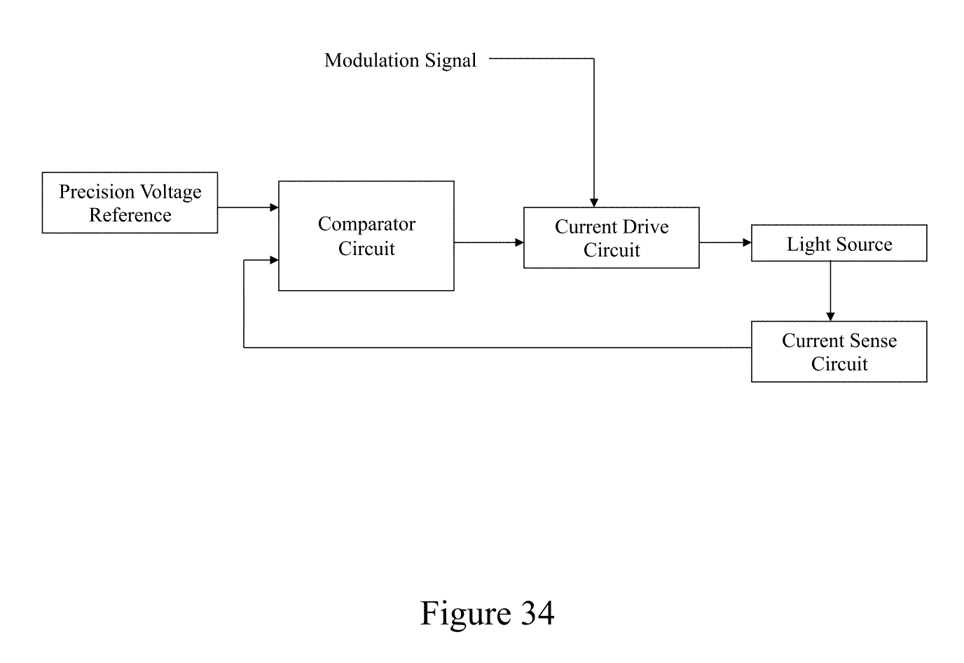

The electrical properties of light sources also influence the emission profile (e.g. wavelength locations of emission) of solid-state light sources. It can be advantageous to stabilize the current and/or voltage supplied to the light source or light sources. For example, the peak emission of diode lasers depends on drive current. For embodiments where the stability of the peak wavelength is important, the stability of the drive current becomes an important Fig. of merit. In such cases, an electronic circuit can be designed to supply a stable drive current to the diode lasers. The complexity and cost of the circuit can depend on the required stability of the drive current. FIG. 34 shows a current drive circuit suitable for use with the present teachings. One skilled in the art recognizes that alternative embodiments of current control circuits are known in the art and can also be suitable for the present teachings. Furthermore, some solid-state light sources require control of the drive voltage, rather than drive current; one skilled in the art recognizes that electronics circuits designed to control voltage rather than current are readily available.

In some embodiments, a single solid-state light source, such as a diode laser, is tuned to multiple wavelengths during the course of a measurement. In order to achieve the tuning of the light sources, the circuits shown in FIGS. 33 and 34 can be modified to include the control of the temperature set point and current, respectively. In some embodiments, either tuning temperature or drive current/voltage can be sufficient to realize the desired tuning of the peak emission wavelength. In other embodiments, control of both the temperature and drive current/voltage can be required to achieve the desired tuning range.

Furthermore, optical means for measuring and stabilizing the peak emission wavelength can also be incorporated into the systems described in connection with the present teachings. A Fabry-Perot etalon can be used to provide a relative wavelength standard. The free spectral range and finesse of the etalon can be specified to provide an optical passband that allows active measurement and control of the diode laser peak wavelength. An example embodiment of this etalon uses a thermally stabilized, flat fused-silica plate with partially mirrored surfaces. For systems where each diode laser is required to provide multiple wavelengths, the free spectral range of the etalon can be chosen such that its transmission peaks coincide with the desired wavelength spacing for tuning. One skilled in the art will recognize that there are many optical configurations and electronic control circuits that are viable for this application. An alternate wavelength-encoding scheme uses a dispersive grating and a secondary array detector to encode the diode laser wavelength into a spatial location on the array. For either the dispersive or the etalon-based schemes, a secondary optical detector that has less stringent performance requirements than the main optical detector can be used. Active control can reduce the stability requirements of the diode laser temperature and current control circuits by allowing real time correction for any drift.

In a dispersive spectrometer the effective resolution of a spectroscopic measurement is often determined by the width of an aperture in the system. The resolution-limiting aperture is often the width of the entrance slit. At the focal plane where light within the spectrometer is detected, multiple images of the slit are formed, with different wavelengths located at different spatial locations on the focal plane. Thus, the ability to detect one wavelength independent of its neighbors is dependent on the width of the slit. Narrower widths allow better resolution between wavelengths at the expense of the amount of light that can be passed through the spectrometer. Consequently, resolution and signal to noise ratio generally trade against each other. Interferometric spectrometers have a similar trade between resolution and signal to noise ratio. In the case of a Michelson interferometer the resolution of the spectrum is in part determined by the distance over which a moving mirror is translated with longer distances resulting in greater resolution. The consequence is that the greater the distance, the more time is required to complete a scan.

In the case of the measurement systems of the present teachings, the resolution of the spectrum is determined by the spectral width of each of the discrete light sources (whether a different light source, one tuned to multiple wavelengths, or a combination thereof). For measurements of analyte properties requiring high resolution, a diode laser or other suitable solid-state laser can be used. The widths of the laser's emission can be very narrow, which translates into high resolution. In measurement applications where moderate to low resolution are required, LED's can be suitable as they typically have wider emission profiles (the output intensity is distributed across a wider range of wavelengths) than solid state laser alternatives.

The effective resolution of light sources can be enhanced through the use, or combination of, different types of optical filters. The spectral width of a light source can be narrowed or attenuated using one or more optical filters in order to achieve higher resolution (e.g. a tighter range of emitted wavelengths). Examples of optical filters that are contemplated in embodiments of the present teachings include, but are not limited to: linearly variable filters (LVF's), dielectric stacks, distributed Bragg gratings, photonic crystal lattice filters, polymer films, absorption filters, reflection filters, etelons, dispersive elements such as prisms and gratings, and quantum dot filters.

Another means for improving the resolution of measurements obtained from embodiments of the present teachings is deconvolution. Deconvolution, and other similar approaches, can be used to isolate the signal difference that is present between two or more overlapping broad light sources. For example, two light sources with partially overlapping emission profiles can be incorporated into a measurement system. A measurement can be acquired from a sample and a spectrum generated (via a Hadamard, Fourier transform, or other suitable transform). With knowledge of the emission profiles of the light sources, the profiles can be deconvolved from the spectrum in order to enhance the resolution of the spectrum.

Light homogenizers such as optical diffusers, light pipes, and other scramblers can be incorporated into some embodiments of the illumination/modulation subsystem 100 in order to provide reproducible and, preferably, uniform radiance at the input of the tissue sampling subsystem 200. Uniform radiance can ensure good photometric accuracy and even illumination of the tissue. Uniform radiance can also reduce errors associated with manufacturing differences between light sources. Uniform radiance can be utilized in the present teachings for achieving accurate and precise measurements. See, e.g., U.S. Pat. No. 6,684,099, incorporated herein by reference.

A ground glass plate is an example of an optical diffuser. The ground surface of the plate effectively scrambles the angle of the radiation emanating from the light source and its transfer optics. A light pipe can be used to homogenize the intensity of the radiation such that it is spatially uniform at the output of the light pipe. In addition, light pipes with a double bend will scramble the angles of the radiation. For creation of uniform spatial intensity and angular distribution, the cross section of the light pipe should not be circular. Square, hexagonal and octagonal cross sections are effective scrambling geometries. The output of the light pipe can directly couple to the input of the tissue sampler or can be used in conjunction with additional transfer optics before the light is sent to the tissue sampler. See, e.g., U.S. patent application Ser. No. 09/832,586, incorporated herein by reference.

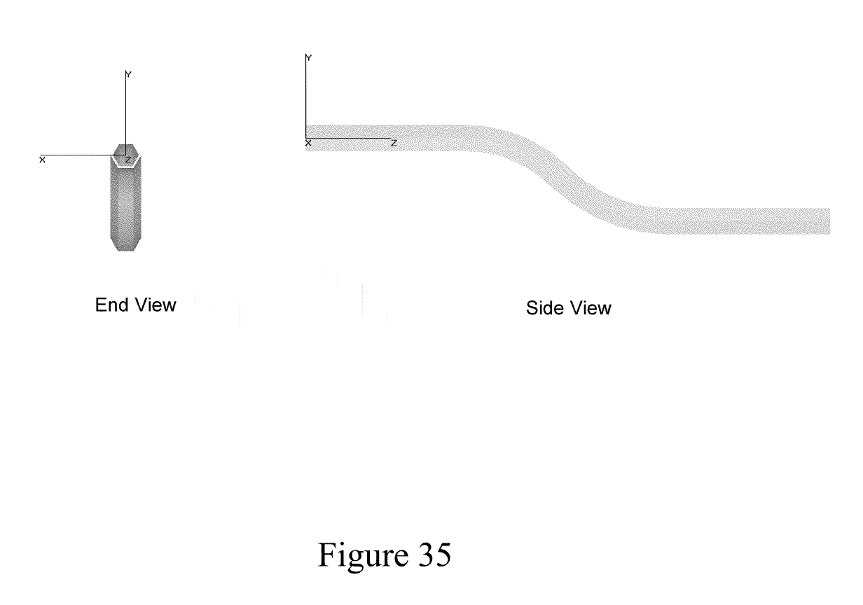

In a preferred embodiment, the radiation homogenizer is a light pipe. FIG. 35 shows a perspective end view and a detail plan view of a light pipe 91 of the present teachings. The light pipe is generally fabricated from a metallic, glass (amorphous), crystalline, polymeric, or other similar material, or any combination thereof. Physically, the light pipe comprises a proximal end, a distal end, and a length there between. The length of a light pipe, for this application, is measured by drawing a straight line from the proximal end to the distal end of the light pipe. Thus, the same segment of light pipe may have varying lengths depending upon the shape the segment forms. The length of the segment readily varies with the light pipe's intended application.

In an example embodiment as illustrated in FIG. 35, the segment forms an S-shaped light pipe. The S-shaped bend in the light pipe provides angular homogenization of the light as it passes through the light pipe. It is, however, recognized that angular homogenization can be achieved in other ways. A plurality of bends or a non-S-shaped bend could be used. Further, a straight light pipe could be used provided the interior surface of the light pipe included a diffusely reflective coating over at least a portion of the length. The coating provides angular homogenization as the light travels through the pipe. Alternatively, the interior surface of the light pipe can be modified to include dimples or “microstructures” such as micro-optical diffusers or lenses to accomplish angular homogenization. Finally, a ground glass diffuser could be used to provide some angular homogenization.

The cross-section of the light pipe may also form various shapes. In particular, the cross-section of the light pipe is preferably polygonal in shape to provide spatial homogenization. Polygonal cross-sections include all polygonal forms having three to many sides. Certain polygonal cross-sections are proven to improve spatial homogenization of channeled radiation. For example, a light pipe possessing a hexagonal cross-section the entire length thereof provided improved spatial homogenization when compared to a light pipe with a cylindrical cross-section of the same length.

Additionally, cross-sections throughout the length of the light pipe may vary. As such, the shape and diameter of any cross-section at one point along the length of the light pipe may vary with a second cross-section taken at a second point along the same segment of pipe.

In certain embodiments, the light pipe is of a hollow construction between the two ends. In these embodiments, at least one lumen or conduit may run the length of the light pipe. The lumens of hollow light pipes generally possess a reflective characteristic. This reflective characteristic aids in channeling radiation through the length of the light pipe so that the radiation may be emitted at the pipe's distal end. The inner diameter of the lumen may further possess either a smooth, diffuse or a textured surface. The surface characteristics of the reflective lumen or conduit aid in spatially and angularly homogenizing radiation as it passes through the length of the light pipe.

In additional embodiments, the light pipe is of solid construction. The solid core could be cover plated, coated, or clad. Again, a solid construction light pipe generally provides for internal reflection. This internal reflection allows radiation entering the proximal end of the solid light pipe to be channeled through the length of the pipe. The channeled radiation may then be emitted out of the distal end of the pipe without significant loss of radiation intensity.

The faceted elliptical reflector is an example of an embodiment of the present teachings that produces only part of the desired characteristics in the output radiation. In the case of the faceted reflector 140, spatial homogenization is achieved but not angular homogenization. In other cases, such as passing the output of the standard system through ground glass, angular homogenization is achieved but not spatial homogenization. In embodiments such as these, where only angular or spatial homogenization is produced (but not both) some improvement in the performance of the spectroscopic system may be expected. However, the degree of improvement would not be expected to be as great as for systems where spatial and angular homogenization of the radiation are simultaneously achieved.

Another method for creating both angular and spatial homogenization is to use an integrating sphere in the illumination system. Although common to use an integrating sphere for detection of light, especially from samples that scatter light, integrating spheres have not been used as part of the illumination system when seeking to measure analytes noninvasively. In practice, radiation output from the emitter could be coupled into the integrating sphere with subsequent illumination of the tissue through an exit port. The emitter can also be located in the integrating sphere. An integrating sphere will result in exceptional angular and spatial homogenization but the efficiency of this system is significantly less than other embodiments previously specified.

Other modifications can be made to the present disclosed system to accomplish desired homogenization of light. For example, the light source can be placed inside the light pipe in a sealed arrangement that would eliminate the need for the reflector. Further, the light pipe can be replaced by an integrator, wherein the source is placed within the integrator. Further, the present system can be used in non-infrared applications to achieve similar results in different wavelength regions depending upon the type of analysis to be conducted.

In some embodiments of the present teachings, the determination of hydration state or total body water can be comprised of the measurement of more than one analyte. For example, collagen and water are the primary components of skin tissue and quantitative values of both would allow determination of water to collagen ratio. The ratio could exhibit a more robust relationship to the hydration state of water, as it could be less susceptible to differences in optical properties within a person over time and between people.

In some embodiments of the present teachings, there noninvasive analyte measurement system can measure additional parameters beyond water concentration, as they are indicators of dehydration, and overall hydration state in the body. Examples of useful analytes include, but are not limited to, lactic acid, lactose, lactate, or combinations thereof. These, and other similar analytes, can indicate the current status of the body in a manner beyond what water concentration can provide alone. For example, determination of lactic acid or lactate could indicate if strenuous activity had occurred as well as how strenuous the activity was. Such information could be of significant utility to athletes and the military, as it would aid in the optimization of training regimens.

In some embodiments, the noninvasive measurement device can measure additional analytes of interest in diagnosing the overall well being of a patient. For example, cholesterol, protein concentrations, and other analytes can be useful in monitoring the health of individuals over time and prevent maladies. Such sensors would provide a health “panel” of information to the user and/or medical treatment professionals.

A challenge in the noninvasive measurement of hydration, TBW, or water concentration is that there is no single level or range of hydration that is correct, in an absolute sense, for all people as a person's ideal hydration state depends on their demographics and lifestyle. As a result, the manner in which embodiments of the present teachings report the results to the users is an important consideration. In some embodiments, the noninvasive device reports an absolute TBW, water concentration, or hydration state. In these embodiments, the user would then interpret the results based on their needs and experience with the sensor. In other embodiments, the sensor would report results that are relative to a given person's normal hydration state. As a result, the user would immediately know that he/she was higher or lower than their normal hydration level and to what degree. One skilled in the art recognizes that both approaches are equally suitable for the present teachings and that a sensor can provide both types of information or allows the user to select which approach is preferred.

The present teachings envision multiple different form factors that enable use in a variety of environments and for a variety of purposes. For example, a tabletop device that measures the forearm, finger, other part of the body, or a combination thereof is suitable for home, office, or medical facility use. These devices can be wall powered, battery powered, or both. In situations where more than one device is in use, the devices can be networked to each other or to a local or remote facility such that data can be shared and/or backed up between the devices.

In other embodiments, the spectroscopic hydration sensor is packaged as a small, wearable, battery operated device that could be worn by athletes, soldiers, the elderly, or any other group with a reason to be concerned about their hydration levels. The sensor can also be incorporated into suitable clothing or equipment. Whether worn, or included in clothing, the sensor can be integrated into the activities of the user and provide constant or semi-constant measurements of hydration state and well being to the user and/or remote monitoring facility or station.

In some embodiments of the present teachings, the noninvasive hydration device can communicate with other systems. These systems can be within the same physical packaging or in one or more separate packages. The packages can be collocated co-located with the noninvasive hydration device or separate. The systems that the noninvasive device can communicate with can also include other noninvasive hydration devices. Communication between the noninvasive hydration device and other systems can be wired, wireless, or a combination thereof. Communication between the hydration device and one or more other systems can be accomplished by, as examples, a high speed serial link, wireless, cellular, internet, phone line, satellite, Ethernet, USB, blue tooth, I2S, I2C, CAN, RS232, cell phone service, or any other form of communication or communication protocol, or combinations thereof.

In some embodiments of the present teachings, information obtained from a noninvasive hydration device is communicated to a phone or to an application on a smart phone. In other embodiments, the noninvasive hydration sensor is integrated within a phone, tablet, music player, or similar consumer device. In some embodiments, one or more noninvasive hydration devices can be integrated into clothing or other work equipment. Information obtained from the integrated devices can be communicated to other systems including those that are also integrated into the same or different clothing and equipment. In some embodiments the hydration sensor can communicate information to a remote or “cloud” storage location. The data can then be accessed by multiple types of devices such as phones, computers, and tablets.

The information obtained from one or more noninvasive hydration devices can be communicated via a variety of means into electronic medical systems, medical records, or a combination thereof. The communication of information obtained from a noninvasive hydration sensor allows the contemplation of many business models including data services such as monthly or similar subscription fees, pay per use, payment for measurement “credits”, payment for data analysis and infometrics, or a combination thereof. Integrated into clothing, central or “cloud” storage server, data service, medical system or medical records. The noninvasive hydration device can communicate many types of information including, but not limited to, hydration results, device status, warnings, errors, databases, quality control metrics, outlier metrics, need for service, or a combination thereof.

Several hydrations studies on humans have been performed using embodiments of the present teachings. The data collected from the human studies was analyzed for hydration state based on the knowledge that human skin tissue is primarily comprised of water and collagen. As a result, to a first order, if water concentration in a given volume of skin were to increase, the corresponding collagen volume would correspondingly decrease. Thus the water to collagen ratio can provide a means for assessing the hydration state of the skin. The spectra of pure water and collagen were collected independently from the spectra measured from humans and used in several ways to determine the water/collagen ratio in the measurements acquired in the human studies.

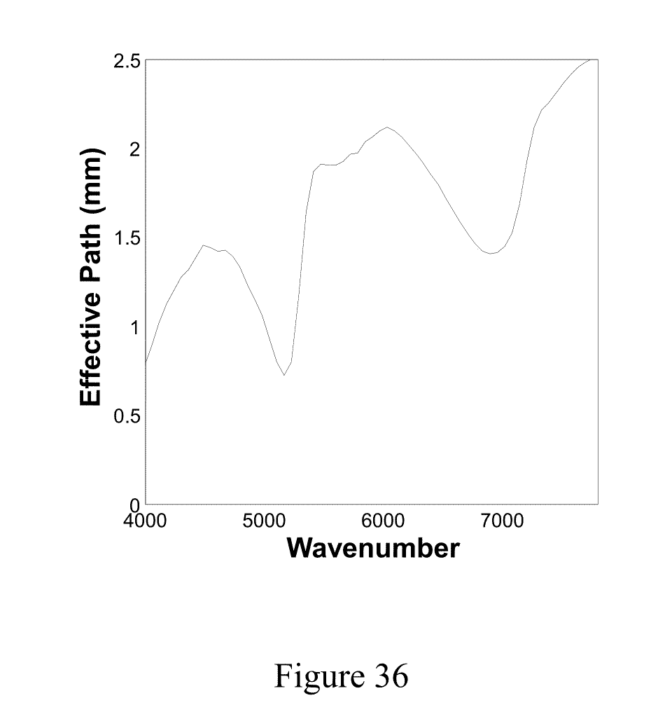

The human studies used an embodiment of the present teachings that was designed to measure human skin tissue. The noninvasive hydration device was optimized for spectral measurements in the 1,200 nm to 2500 nm wavelength range and used a ceramic blackbody light source in the illumination subsystem 100. A gold-coated hexagonal cross-section light pipe collected and homogenized the light from the ceramic blackbody light source and introduced it to the input of the sampling subsystem 200. The sampling subsystem 200 was comprised of the design shown in FIG. 18. The design of the tissue interface of the sampling subsystem 200 was such that the measured spectra had an average effective pathlength (FIG. 36) through skin sufficient to interrogate the dermis of the skin. In general, the depth of penetration for this embodiment of the teachings was approximately 0.7-1 mm, which indicates that the water interrogated by the system predominantly resides in the dermis of the skin tissue. The light collected from the skin was homogenized by the sampling subsystem using a gold-coated hexagonal cross-sectioned light pipe. The output of the light pipe delivered the homogenized light to the input of the spectrometer subsystem 300. The spectrometer subsystem 300 is incorporated a Michelson geometry Fourier Transform interferometers (as shown in FIG. 24). The output of the spectrometer subsystem 300 directed the light to an extended InGaAs photodetector (part of the data acquisition subsystem 400). The data acquisition subsystem then filtered and digitized the collected light and generated interferograms, which were then converted to intensity versus wavelength spectra for further use by the computing subsystem 500 and hydration determinations.

Hydration measurements from the human studies were obtained using 3 different methods. First, PCA was used to find patterns in the collected human spectra. The multi-dimensional (multiple measurements at multiple wavelengths) spectral data is decomposed into scores and loadings for a relatively small number of orthogonal factors; the scores give information about correlations and trends in the sample space while the factors provide insight into the correlations and trends in the spectra/wavelength space. Since the study was focused on trends in the water/collagen ratio and hydration, the PCA factors were examined in order to identify principle components with inversely related water and collagen features. The principle component with peaks from both the water and collagen pure component spectra were retained and the corresponding scores were used as indicators of water trends in the body.

Second, the pure spectra of water and collagen (called pure components) that were collected independently from the human studies were used to fit each acquired human spectrum via nonlinear least squares regression. In contrast to the PCA approach, fitting the spectra using the known pure component spectra can provide an estimate of water and collagen concentrations that has more physical meaning. The nonlinear regression was performed on each measured tissue spectrum independently and used the pure component spectra of water and collagen to estimate their associated concentrations as well as the effective path length the measured light took through the skin. A ratio of the water and collagen estimates was calculated from the spectral fits and this measure was evaluated for possible trend information.

Third, synthetic models were used to provide evidence towards the presence of a water/collagen signal in subject spectra. Two partial least squares (PLS) regression models are built using synthetically derived human spectra with known quantities of water and collagen together with a wide range of scattering characteristics. One PLS model was used to determine water concentration, while the second determined collagen concentration. The ratio of water to collagen was then calculated and evaluated as a marker for hydration changes.

The synthetic data set was generated using proprietary tools that incorporated tissue scattering and absorbance properties and structural information, instrument effects, and noise. The spectral scattering characteristics were created using path length distributions derived from a Monte Carlo simulation of human tissue and used literature values for the scattering and absorption coefficients and a Henyey-Greenstein phase function with anisotropies ranging from 0.8 to 0.95. Water and collagen were the only analytes whose concentration varied in the synthetic spectra, and a full factorial design was used to ensure that those concentrations would cover the entire space of the actual subject data.

Once the estimates from each method were derived from the spectra acquired in the human studies, the estimates were used to investigate hydration trends in the data and compare them to reference hydration measurements (in these studies, the subject's weight over time was used as the hydration reference). In some cases, a moving average was calculated for each subject for each of the different estimates in order to simplify visualization of the trends. Given that it is an approach that could be employed during deployment of a noninvasive hydration monitor, it is a valid approach to take. A 21 point window was used.

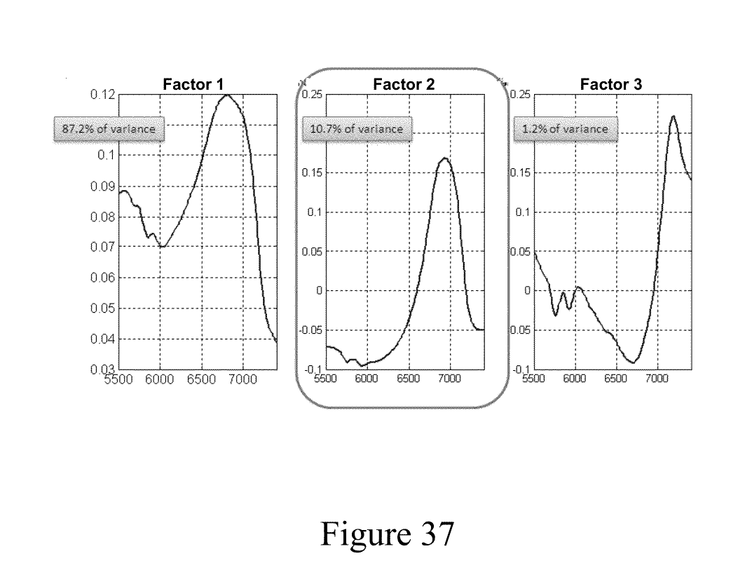

In one human study, data was collected on approximately 30 individuals over an 18-month period using one noninvasive hydration device. PCA of the study spectra produced the first three principle components whose factors are displayed in FIG. 37. The second principle component in particular shows a water/collagen signal. This factor accounts for 10.7% of the variance in the spectral data.

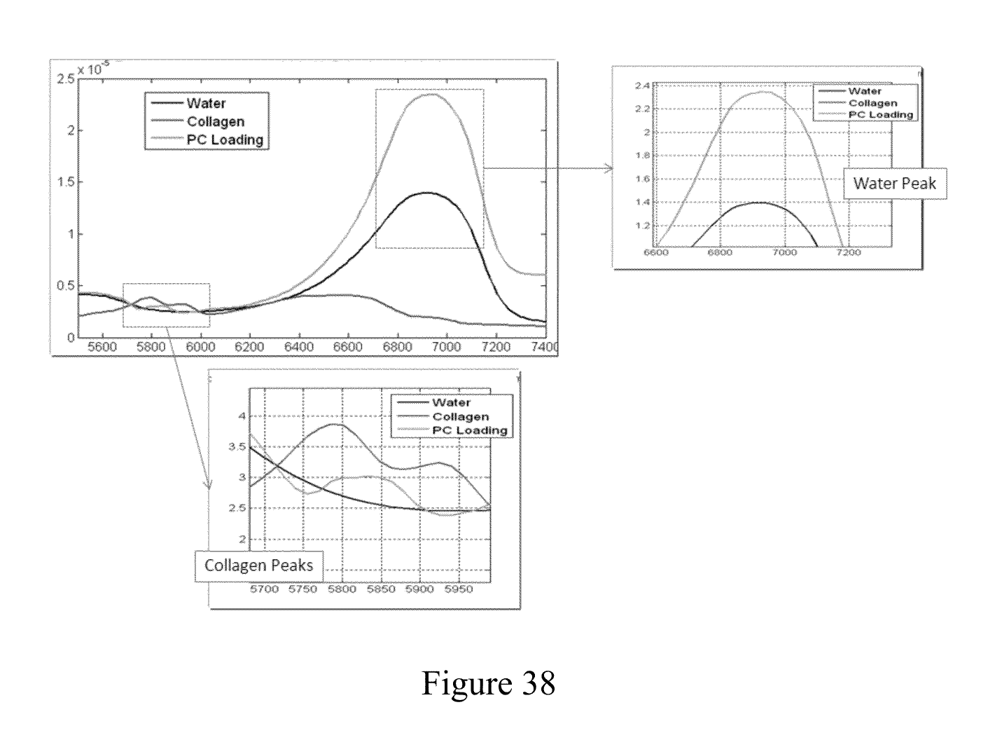

In comparing the peaks of the second principle component with those of the pure component spectra for water and collagen (FIG. 38), it can be seen that the peak at 6900 cm⁻¹ corresponds closely to the water peak in the pure component spectrum. Furthermore, the negative peaks in the 5800 cm⁻¹ region closely match those of collagen in its pure component spectrum. This suggests that this principle component relates to trends in the data when there is an increase in water with a corresponding decrease in collagen, which in turn relates to hydration trends in the skin.

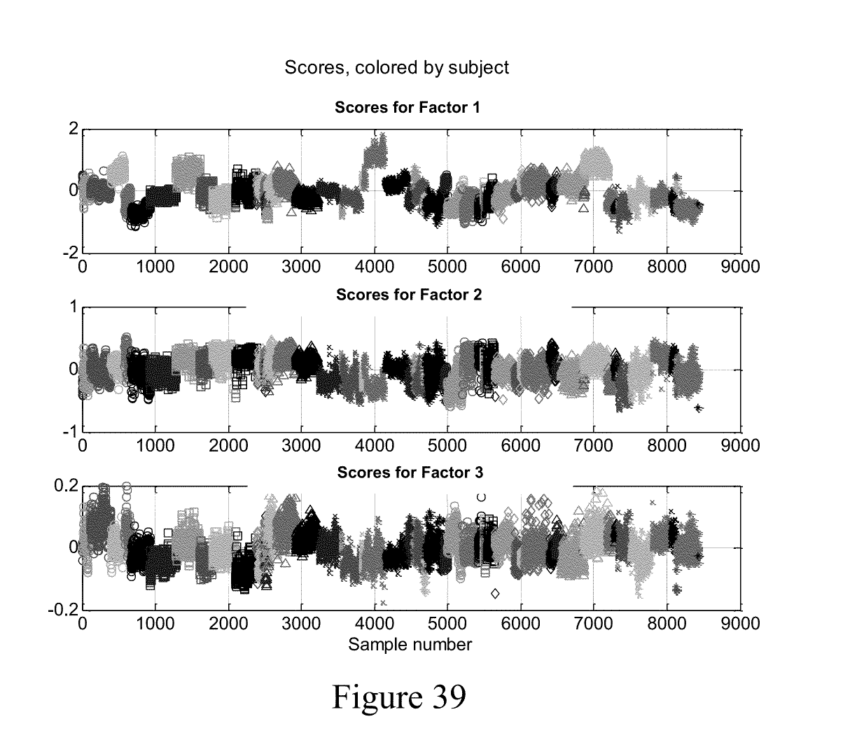

The scores corresponding to factor 2 therefore display how hydration changes for each subject over the course of the study. FIG. 39 shows the scores for all study participants (each color/symbol combination represents a different subject) for the first three principle components. A general interpretation of FIG. 39 is that average differences in scores between subjects indicate inter-subject differences in water/collagen ratio while the systematic variation within each subject's scores is indicative of changes in hydration over time.

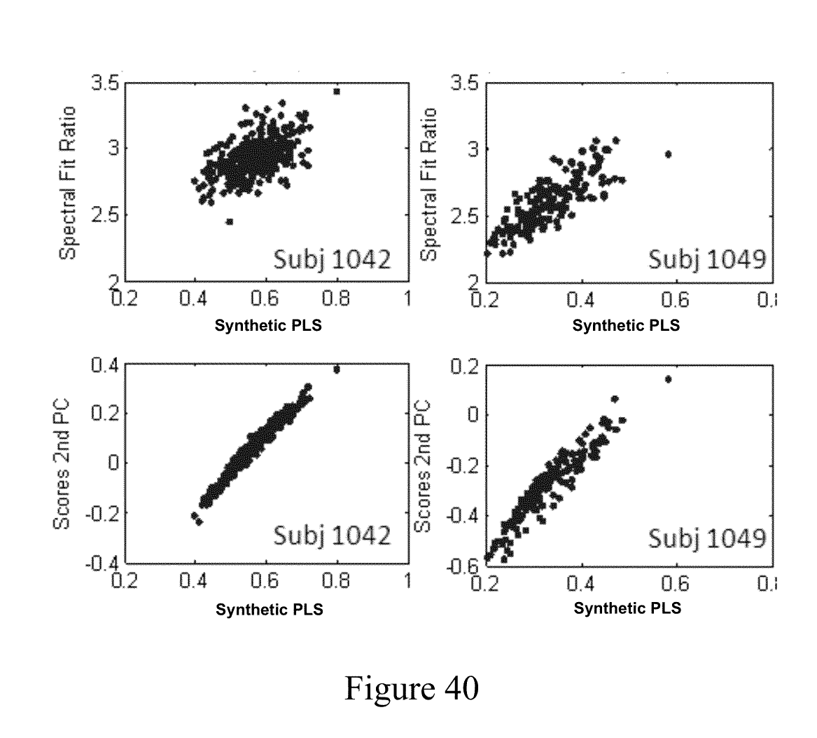

Determinations of water/collagen ratio were also obtained from the human study data using the nonlinear regression and synthetic PLS model approaches mentioned above. The synthetic spectra were created with the intention of covering a large spectroscopic space that encompassed the scattering and concentration characteristics encountered in the in vivo study spectra. In FIG. 40, the correlation between the PCA and synthetic PLS hydration measurements is shown for two different subjects. Examination of FIG. 40 shows that the results from each method are correlated which is indicative that they are all leveraging similar spectroscopic information when determining their respective water to collagen. This is reassuring that the methods are indeed measuring a signal related to hydration and not random noise present in the data.

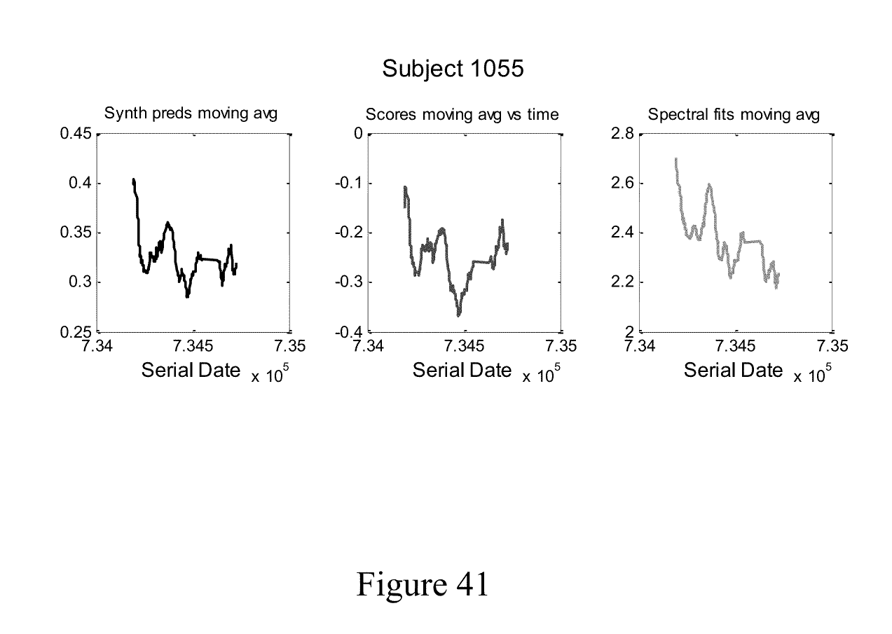

FIG. 41 displays the trends versus serial date (e.g. time) that were obtained from each of the approaches for a single study participant. A couple of observations can be made; the first is that this signal does indeed vary in a regular manner over the course of data collection. The second is that the three methods for determining hydration from the spectra display similar, though not identical, trends for the subject. This supports the hypothesis that all three approaches are measuring the same physical phenomenon.

Two exercise studies were executed in order to further examine the performance of the embodiment of the noninvasive hydration device of the present teachings. Changes in body weight are known to be an accurate means of quantifying total body hydration over a short time frame, particularly during exercise studies. Weight loss therefore corresponds to fluid loss, and it was used as the reference for the exercise studies reported herein. Body weight was used as a surrogate measurement for total body water (TBW). Subjects were weighed and measured using the embodiment of the present teachings prior to an intensive running session, during which they were again weighed and noninvasively measured using the present teachings. Weights and noninvasive measurements were again taken after the exercise was completed, followed by fluid intake and additional measurements. Two separate studies took place; the first was a dry run and involved a single individual. In the second experiment there were three subjects, two of whom followed the same format as the dry run and one of whom did not consume fluid for an hour after completing the run. The three previously discussed methods for obtaining water to collagen ratios were applied to the acquired exercise study data.

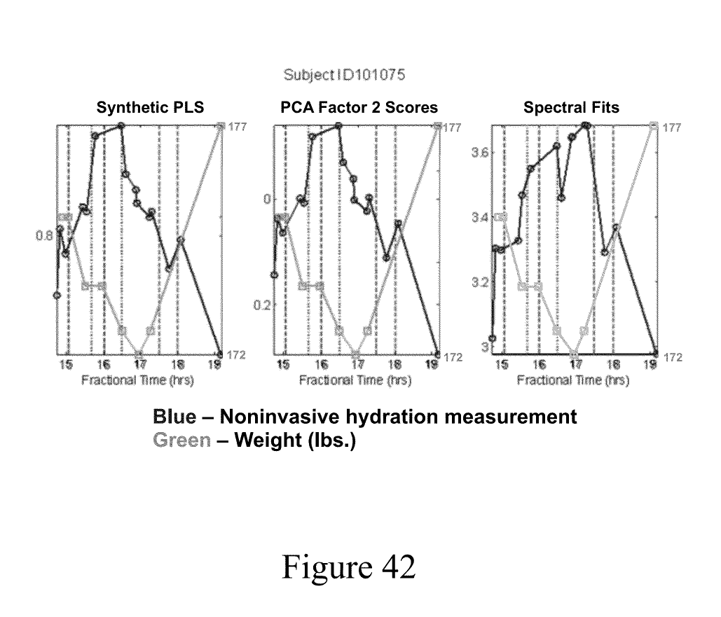

FIG. 42 shows both the reference weights and the water/collagen ratio estimates versus time for one subject. Body weight decreases from the start of exercise to exercise completion: this is expected as fluid is excreted and lost from the body as sweat. Fluid is consumed and weight begins to increase immediately (although the water certainly resides in the stomach for an unknown period of time). The same trend in weight was also seen for the other two subjects. The hydration measurements of this embodiment of the present teachings exhibited the opposite pattern: the water collagen ratio increased throughout the exercise run while the body was dehydrating, and subsequently decreased once fluid was consumed. This is because the skin becomes more perfused with fluid during exercise in order to facilitate sweating and in turn control of body temperature during exercise.

There are important applications where skin hydration is the parameter of interest. For example, cardiovascular disease results in endothelial dysfunction, which is an imbalance between vasodilating and vasoconstricting substances in the endothelium. Research has shown that impaired endothelial vascular signaling leading to endothelial dysfunction is one of the earliest vascular changes in the pathogenesis of cardiovascular disease. Existing methods for examining skin as a surrogate measurement for cardiovascular disease can be complicated as the skin-specific methodologies induce vasodilation and/or vasoconstriction via multiple, and often time redundant, mechanisms. A noninvasive technique such as that of the present teachings could provide a test for endothelial function that would eliminate many of these confounding effects and be a valuable predictor of cardiovascular risk.

In cases where total body water or overall hydration state is more important than skin hydration, alternative embodiments of the present teachings would be used that are designed to measure deeper tissues within the body that reflect TBW and the body's hydration state. The primary means for accomplishing deeper depth of penetration are changing the wavelength of light used by the device (shorter wavelengths are less attenuated by absorbance) and through the design of the sampling subsystem 200 (e.g. increased separation between illumination and collection optical fibers). Furthermore, some embodiments of the present teachings interrogate deeper tissues by using a different technique than absorbance/reflectance spectroscopy such as Raman spectroscopy where the excitation wavelength can be chosen to achieve the desired depth.

In some embodiments of the present teachings, the Fourier Transform interferometer in the preceding embodiment can be replaced with a dispersive spectrometer. An example of a suitable dispersive spectrometer is a Czerny-Turner design with a reflective grating. The output (e.g. focal plane) of the spectrometer subsystem can be aligned with an array detector such as a 256 element InGaAs array. The resulting system has fixed alignment and no moving parts that is advantageous in some applications. Alternatively a single element detector can be used (as with the Fourier Transform interferometer embodiment) and the grating can be rotated during a measurement in order to measure the intensity at the desired wavelengths.

Referring again to the embodiment described in the Noninvasive Hydration Sensor Measurements in Humans section, the ceramic blackbody light source can be replaced in some embodiments by one or more light emitting diodes (LED's). The advantage of the LED's is the ability to eliminate light at undesirable wavelengths or wavelength ranges as well as improve the electrical efficiency of the system and increase its useful life. In these embodiments, the remainder of the system is equivalent to that previously described.

In an example embodiment of the present teachings, a noninvasive water measurement system is comprised of 13 diode lasers that are used to measure 22 discrete wavelengths. In this embodiment, the illumination subsystem and spectrometer subsystem 300 are combined into an illumination/modulation subsystem. In this embodiment, each diode laser is stabilized to a constant temperature. The peak wavelength of each diode laser is controlled based on the circuit shown in FIG. 34 (each diode laser having its own circuit), which also enables the diode laser to be turned On and Off. The specific state (On/Off) of each diode laser at a given time during a measurement is determined by a predetermined encoding (Hadamard, Grey, similar schemes, or a combination thereof) scheme. In example embodiments incorporating solid state light sources a Hadamard matrix is a pattern of On/Off states versus time for each diode laser that is stored in software rather than a physical mask or chopper. This allows the On/Off states stored in software to be conveyed to the electronic control circuits of each diode laser during the measurement.

As several of the diode lasers are responsible for more than one wavelength, a single encoding scheme that incorporates all wavelengths can be difficult to achieve. In this case, a combination of scanning and encoding can allow all target wavelengths to be measured. In the present embodiment, all diode lasers are tuned to their 1st target wavelength (for those with more than 1 target wavelength) and an encoding scheme is used in order to achieve the associated multiplex benefit. The relevant diode lasers can then be tuned to their subsequent target wavelengths and additional encoding schemes used. Diode lasers with only 1 target wavelength can be measured in either or both groups or divided among the groups.

Furthermore, the groups can be interleaved in time. For example, for a 2 second measurement, the first group can be measured for the 1st second and the 2nd group for the 2nd second. Alternatively, the measurement can alternate at 0.5-second intervals for 2 seconds. The measurement times do not need to be symmetric across the groups. For example, it can be desirable to optimize signal to noise ratio by weighting the measurement time towards one or the other group. One skilled in the art recognizes that many permutations of measurement time, balancing the number of groups, balancing the ratio of scanning to encoding, and interleaving are possible and contemplated in the embodiments of the present teachings. Furthermore, one skilled in the art recognizes that a variety of embodiments exist with differing numbers of solid-state light sources and target wavelengths and that all are suitable for the purposes of the present teachings.

In some embodiments the output of the diode lasers are combined and homogenized using a hexagonal cross-sectioned light pipe. In some embodiments, the light pipe can contain one or more bends in order to provide angular homogenization in addition to spatial homogenization. Regardless, at the output of the light pipe, the emission of all diode lasers preferably spatially and angularly homogenized such that all wavelengths have substantially equivalent spatial and angular content upon introduction to the input of the sampling subsystem 200.

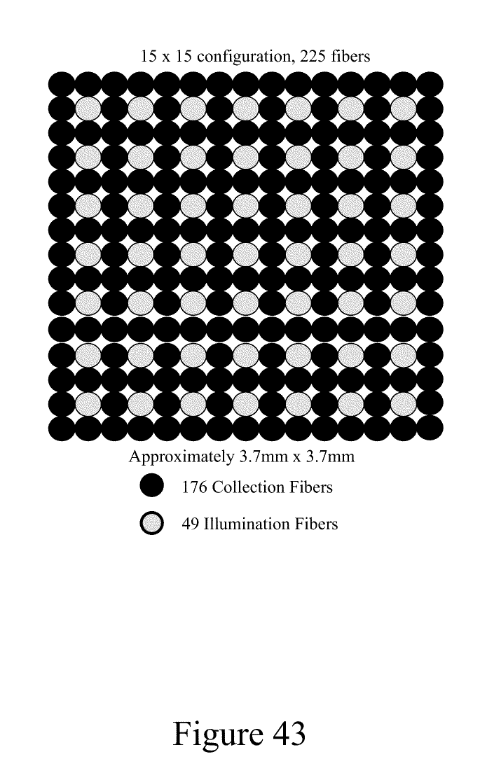

The homogenized light is introduced to the input of an optical probe. In the example embodiment, the input is comprised of 225, 0.37NA silica-silica optical fibers (referred to as illumination fibers) arranged in a geometry consistent with the cross section of the light homogenizer. The light is then transferred to the sample interface. The light exits the optical probe and enters the sample, a portion of that light interacts with the sample and is collected. In the present preferred embodiment, the collection fibers are 0.37 NA silica-silica fibers. FIG. 43 shows the spatial relationship between the illumination and collection fibers at the sample interface.

The optical probe output arranges the collection fibers into geometry consistent with the introduction to a homogenizer. For the example embodiment, the homogenizer is a hexagonal light pipe. The homogenizer ensures that the content of each collection fiber contributes substantially equally to the measured optical signal. This can be important for samples, such as human tissue, that can be heterogeneous in nature. The output of the homogenizer is then focused onto an optical detector. In the present preferred embodiment, the optical detector is an extended InGaAs diode whose output current varies based upon the amount of incident light.

The processing subsystem then filters and processes the current and then converts it to a digital signal using a 2-channel delta-sigma ADC. In the example embodiment, the processed analog detector signal is divided and introduced to both ADC channels. As the example embodiment involves laser diodes with multiple wavelength groups (e.g. some lasers have more than one target wavelength), a Hadamard transform is applied to the spectroscopic signal obtained from each group and the subsequent transforms combined to form an intensity spectrum. The intensity spectrum is then base 10 log transformed prior to subsequent water concentration determination.

The example embodiment is suitable for either “enrolled” or “walk-up/universal” modalities as well as applications combining water with other analyte properties such as lactose or lactate. Furthermore, any of the discussed modalities or combinations can be considered independently or combined with the measurement of a biometric property.

In another example embodiment, 50 wavelengths are measured using 24 diode lasers. As some of the laser diodes are responsible multiple target wavelengths, there are multiple wavelength groups, each with its own Hadamard encoding scheme. The remainder of the system parameters, including the optical probe design, light homogenizers, detector, and processing is identical to the earlier described preferred embodiment.

In another example embodiment, the illumination subsystem and spectrometer subsystem are combined into an illumination/modulation subsystem. The illumination/modulation subsystem is then combined with the sampling subsystem to form an integrated sampling subsystem in order to provide a compact noninvasive hydration device suitable for integration into clothing, wearable equipment, or electronics such as cell phones, tablets, computers, or any other device that is used by humans. As the emission areas of solid-state light sources such as diode laser can be on the order of several microns in diameter, they can be arranged at the sample interface where the human interacts in geometries and orientations similar to those of optical fibers. In this example, 4 laser diodes of different wavelengths are arranged around a single element InGaAs detector. An optically transparent material such as a fused silica window, microlens array, or other suitable material is then placed over the laser diodes and detector. This material serves as the sample interface and prevents damage to the laser diodes and detector. The laser diodes can be measured sequentially, an encoding scheme, or a combination thereof. One skilled in the art recognizes the significant reduction in instrument size and complexity offered by the integrated sampling subsystem offers significant commercial advantages. Furthermore, one skilled in the art recognizes that a variety of integrated sampling subsystem embodiments exist with differing numbers of solid state light sources and target wavelengths and that all are suitable for the purposes of the present teachings.

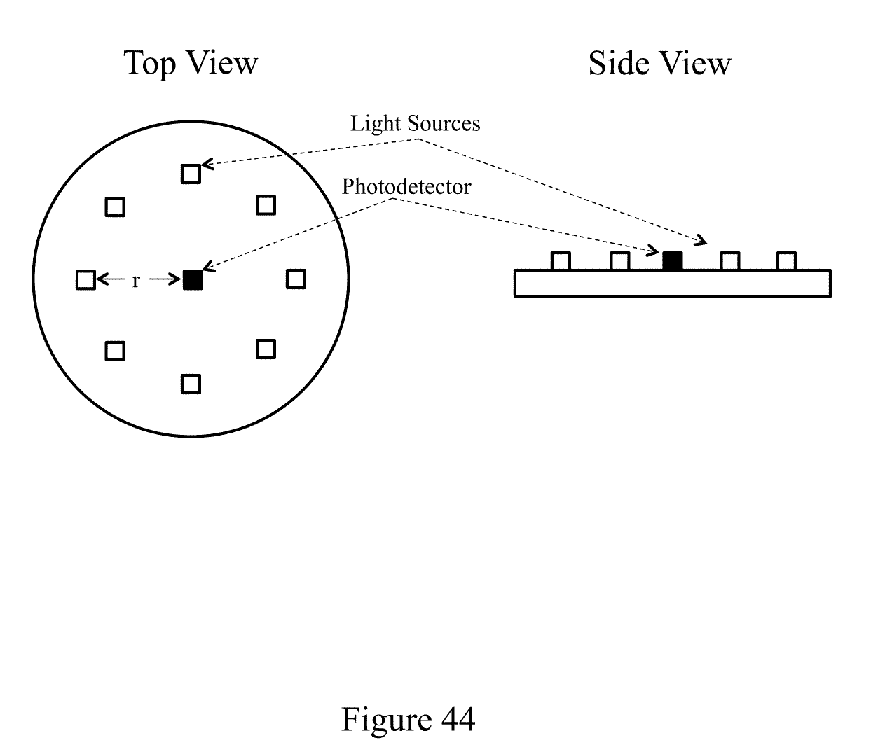

FIGS. 44-46 show embodiments of the present teachings that exploit the special case “Illumination/Modulation Subsystem (100)”. An advantage of these embodiments is that the illumination/modulation subsystem eliminates the need for a spectrometer. Another advantage of these embodiments is that the arrangement of the light sources and detector eliminate the need for a distinct optical receiver subsystem. In other words, the relative locations and configurations of the light source and photodetector to each other serve the same function as a distinct optical receiver subsystem. As a result, these embodiments combine the functions of all of the subsystems of the present teachings into a simple, compact package.

FIG. 44 shows an embodiment of the present teachings that contains a photodetector surrounded by light sources at a radius “r” from the photodetector. The light sources can be light emitting diodes, diode lasers, or any other suitable light source. Each light source can emit the same wavelength, range of wavelengths, or different wavelengths and can define a first illuminator optical axis. For example, the 8 light sources shown in FIG. 44 could be comprised of 4 diode lasers emitting light at substantially one wavelength and 4 diode lasers emitting light at substantially one wavelength, but distinct from the wavelength emitted by the first 4 diode lasers. Alternatively, each light source could emit a different wavelength of near-infrared light. Furthermore, the radius “r” between each light source and the photodetector do not need to be the same. This allows the arrangement of the light sources relative to the detector to alter the propagation of light through the tissue on a wavelength by wavelength basis. Furthermore, the number of light sources can range from one to as many will fit within the physical confines of the sensor. The Optical receiver can additionally define a second optical receiver axis. Optionally, the receiver axis can be a non-parallel angle with respect to the light source first optical axis.

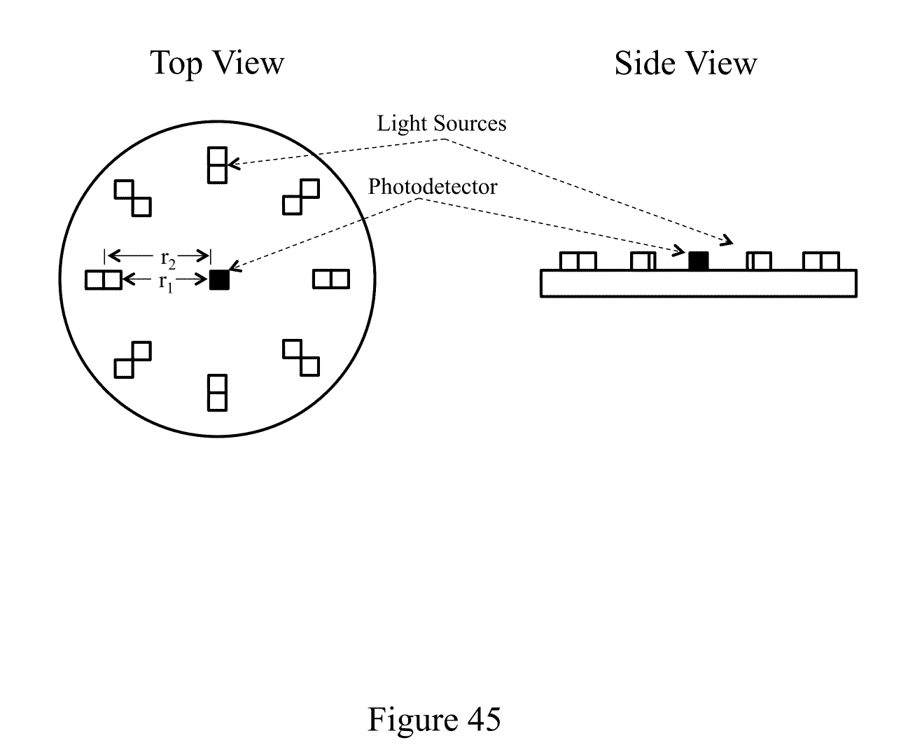

FIG. 45 shows a similar embodiment to that shown in FIG. 44 where a 2^{nd} ring of light sources has been added. The second ring can be measured simultaneously with those of the first ring, or treated as a distinct “channel” as mentioned earlier in this disclosure. Such arrangements can be used to compensate for tissue surface effects such as topical interferences or compensate for unwanted light that has travelled through undesirable shallow tissues. Additional rings can also be used to increase the signal to noise ratio of the system due to the additional light emitted by the larger number of light sources.

It is recognized by one skilled in the art that the range of angles emitted by the light sources can be controlled within the light sources or by the use of additional optical components such as lenses, coatings, waveguides or homogenizers. The range of angles accepted by the photodetector can be also be similarly controlled. In some embodiments of the present teachings, such control of the angles emitted and collected is used to preferentially interrogate tissues that represent the hydration state of the individual. The angle of the light sources and the photodetector relative to the tissue surface can also be used to control the trajectory of light as it propagates through the tissue and can therefore also impact the part of the tissue that is interrogated.

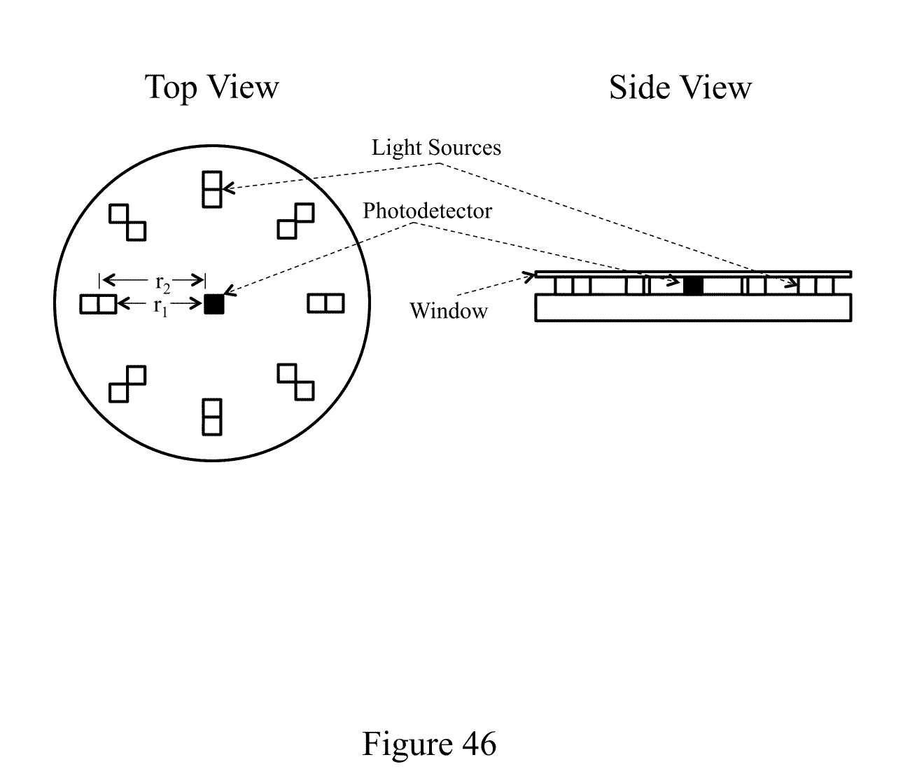

FIG. 46 shows an embodiment that incorporates an optical window between the tissue and the optical components (light sources and photodetector). In some embodiments, the window provides a protective layer that prevents contamination or failure of the light sources or photodetector due to the presence of materials such as interferences or sweat on the tissue surface. The window can be any material (glass, quartz, fused silica, sapphire, plastic, etc.) that sufficiently transmits the wavelengths of light used by the embodiment.

### Embodiments of Disposables for Noninvasive Hydration Sensors

In some embodiments of the present invention one or more disposable elements of the system are contemplated. The “disposable” or limited use elements can relate to ensuring patient/user safety, system calibration or system verification of the noninvasive hydration sensor, and cosmetic enhancement of the sensor. Furthermore, it is recognized that disposables can be an advantageous source of revenue.

An aspect of non-invasive analyte measurements, such as hydration, is the safety of the subjects during the use of the device. In multi-user environments it can be necessary to provide methods of mitigating device contamination or transfer of pathogens between subjects. It can be desirable to use disposable cleaning agents and/or protective surfaces in order to protect each subject and prevent fluid or pathogen transfer between subjects. For example, in some embodiments an isopropyl wipe can be used to clean each subject's sampling site and/or the sampling subsystem surface prior to measurement. In some embodiments, a disposable thin film of material such as ACLAR can be placed between the sampling subsystem and the subject prior to each measurement in order to prevent physical contact between the subject and the instrument. In some embodiments, both cleaning and a film could be used simultaneously. As mentioned in the sampling subsystem portion of this disclosure, the film can also be attached to a positioning device and then applied to the subject's sampling site. In this embodiment, the positioning device can interface with the sampling subsystem and prevent the subject from moving during the measurement while the film serves its protective role.

In some embodiments, such as emergency medicine, it can be advantageous to have an optical probe that is disposable. An optical probe can be used for a given patient and then discarded/destroyed upon discharge (or when hydration measurements are no longer necessary for the patient). Subsequent patients can then get their own optical probe. This improves the hygiene of the measurement as well as offering recurring revenue from the disposable.

In addition to disposables to ensure subject safety, disposable calibration check samples can be used to verify that the instrument is in proper working condition. In many commercial applications of analyte measurements, the status of the instrument must be verified to ensure that subsequent measurements will provide accurate concentrations or attribute estimates. The instrument status is often checked immediately prior to a subject measurement. In some embodiments, the calibration check sample can include the analyte of interest. In other embodiments, the check sample can be an environmentally stable and spectrally inert sample, such as an integrating sphere or other diffuse reflective surface. The check sample can be a gas or liquid that is injected or flowed through a spectroscopic sampling chamber. The check sample can also be a solid, such as a gel, that may contain the analyte of interest. The check sample can be constructed to interface with the sampling subsystem or it can be incorporated into another area of the optical path of the system. In some embodiments, the calibration check sample may be physically integrated into a single disposable element which also includes a protective barrier for hygiene purposes as described above.

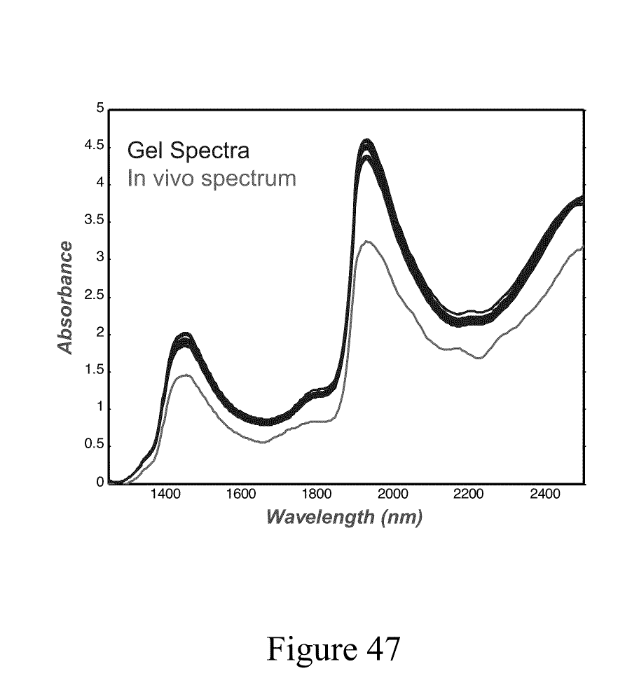

FIG. 47 shows NIR spectra in the 1300 to 2400 nm region obtained from several gels as well as a NIR spectrum obtained from a person.

These examples are meant to be illustrative and are not limiting to the various possible calibration check samples. The calibration check sample can also contain a physical or chemical marker that is included in the sample in order to prevent counterfeit or unauthorized check samples from being used in conjunction with the noninvasive hydration monitor.

Remote and Local Databases, Cloud Data Storage, and Electronic Medical Records

Noninvasive Hydration Device Networking and Communication

Several existing hardware and data transfer mechanisms are useful for the present invention including, but not limited to, Ethernet, USB, wireless (802.11, etc), Zigbee, and Bluetooth. In general, these are all variants of serial communication protocols and can be divided into the general categories of wired and wireless, both are suitable for the present invention. Wired, wireless, or a combination thereof approaches can be used to connect noninvasive measurement devices to local or remote (e.g. potentially long distance) networks. Such connections can be point-to-point, spoke-hub (router), mesh (system to system/peer to peer), or a combination thereof depending on the needs of a given customer, facility, group of facilities, etc.

In some embodiments, wireless protocols can be used. Some example wireless protocols that are useful for connecting one or more systems to a central server include, but are not limited to: wifi, wimax, wireless, and wireless USB. Long range serial FM or AM transmitters can also be used for wireless remote installations requiring data transmissions on the order of a few miles or more. For ease of distributed installation/reconfigurable installations at an industrial facility, a mesh network topology can be implemented where each measurement system of the present invention acts as a message router and provides an automatic self-configuring/self-repairing setup. Commercially available examples of mesh network protocols include Zigbee radios. Other mesh network protocols are known to those skilled in the art and can be suitable for the present invention. In some embodiments of the present invention that require remote installations, cellular communication hardware/protocols can be viable for data transmission. Addition examples include CDMA, satellite, etc.

Regardless of the hardware/connection architecture, it can be advantageous for some embodiments of the present invention to use standard industry communication protocols that include, but are not limited to, RS-232, CAN, RS-422, RS-485, SPI, I2C, and I2S. Depending on the embodiment, more than one such communication protocol can be used. In comparison to many high-speed data transfer applications, the analyte measurement systems of the present invention have comparatively low bandwidth requirements. Due to the low bandwidth requirements, low bandwidth serial protocols can be suitable, and can be especially useful in noisy industrial environments.

In some embodiments, the analyte measurement device or devices can be configured with a hardware interface such as Ethernet, yet require remote connection to a network. In such cases, dedicated adapters can be used to interface the Ethernet port of the analyte measurement device with the desired remote network connection architecture. Some examples of such adapters include, but are not limited to, cell to Ethernet, satellite to Ethernet, cell to USB, satellite to USB, Ethernet to Bluetooth, Ethernet to USB, USB to Bluetooth, and various combinations thereof.

In order to manage multiple configuration options potentially required by various customers, a base communication protocol can be specified (e.g. Ethernet). Off-the-shelf OEM modules can be used to provide conversion of the Ethernet data stream to any of the desired communication protocols mentioned above. In the case of wireless protocols, the adapter hardware can include the requisite radio transmission hardware.

Data Transfer and Centralization

In order to support data privacy and protection regulations as well as customer requirements, some embodiments of the present invention can incorporate data security and encryption that can be implemented at several levels. Secure network protocols, such as SSH, HTTPS, etc., can be implemented which provide for encrypted network communication. Data access rights can be authenticated at the user, server, facility, or other appropriate level via encrypted authentication. Several commercially available schemes exist including, but are not limited to, RSA and AES methods.

For potentially sensitive data (company or user information, billing information, etc.) the local data storage on the analyte measurement device can also be encrypted. For transmission/distribution of this data, the encrypted data can be sent directly. By not locally decrypting prior to transmission, this data retains some level of protection even if the secure network transmission protocol were to become compromised. This type of layered security approach can be important when contemplating the use of third party communication hardware/software as part of the communication pipeline (e.g. reliance on cellular phone carriers for data transmission).

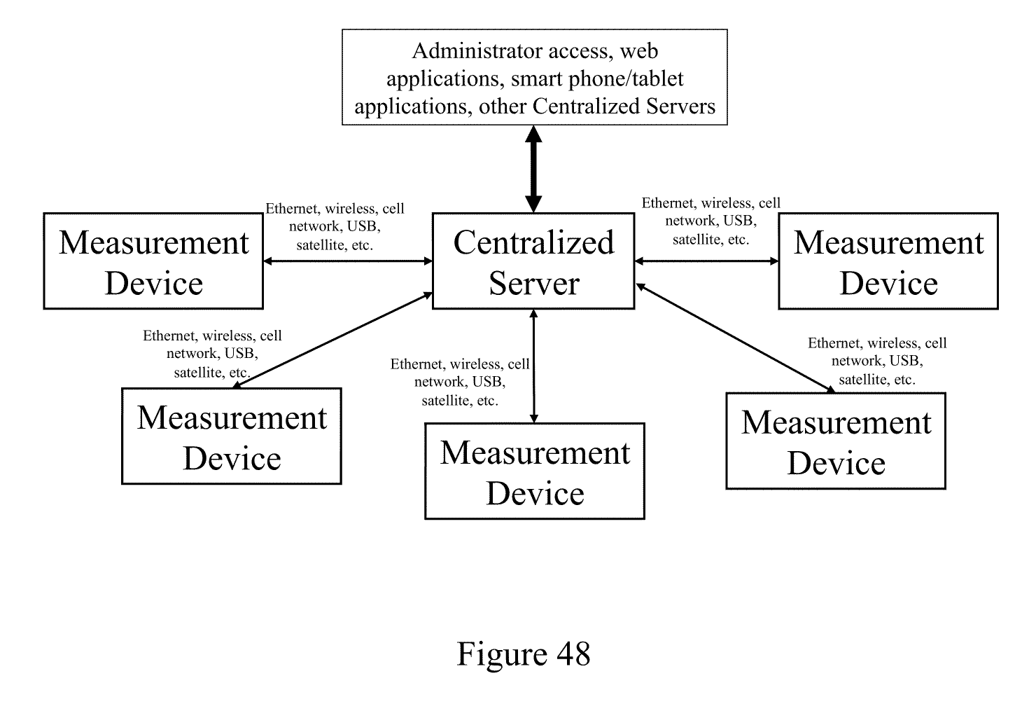

FIG. 48 shows a diagram of several devices connected to a centralized server where each device can connect with the same, or different, approach such as, but not limited to, Ethernet, USB, wireless, cell network, RS 232, satellite. The centralized server is also connected to administrators, whether local or remote, through computer applications, web applications, phone/tablet applications, or other means of interfacing such as keyboard, mice, etc. In order to prevent momentary network connectivity problems from resulting in loss of critical operations, the analyte measurement devices of the present invention can be able to operate in stand-alone mode for reasonable periods of time. Within a given location, each analyte measurement device can maintain a local copy of any requisite information. The data replication and updating can make use of modern database designs which are built to allow data storage and retrieval to be distributed across multiple data servers. Under this type of design, information can be “pushed” to the central data server(s) at the time a measurement is performed. At pre-defined, reasonable intervals, any or all of the analyte measurement devices can query the central data server for updates and new information. Momentary loss of network connectivity for any sensor does not result in as a given analyte measurement device has all od the information and data it needs based on the most recent query to the central database. Once network access is restored, any updated test results and information and data can be uploaded to the central data repository.

There are several types of data that can, depending on the embodiment, indicate transfer between devices or between a device and a centralized database. In some embodiments, the noninvasive measurement devices can be administered and monitored by a specific individual or group of individuals (e.g. nurses or physicians). In such cases it can be desirable to transfer test results to a centralized location (either onsite or offsite). The transferred results and associated information is then available for examination by the designated staff. For example, in medical facilities the results can be transferred directly to electronic patient records in addition to or in lieu of onscreen results. These examinations can range from individual actions based on the measurement result to the generation of reports and statistics that summarize the outcomes of the testing on a routine (hourly, daily, monthly, etc.) basis. Some examples of information that can be important to transfer are the hydration or other analyte measurement results, the associated time and date, the user/patient ID of the person tested, and instrument status. These types of information are intended to be demonstrative for the purposes of the present invention. Those skilled in the art recognize that other types of information acquired or recorded by a noninvasive measurement device could also be important or desirable to transfer.

In some embodiments spectroscopic data from any networked device can also be transferred. Such transfers can be automatic or at the request of a local or remote administrator. In some embodiments the spectroscopic data can be useful for diagnostic and troubleshooting purposes as well as to establish a historical record of spectroscopic measurements acquired. The transferred data can be raw (e.g. digitized photodetector values), processed (raw signals converted to intensity versus wavelength spectra) as well as time resolved (most spectroscopic systems can yield multiple intensity versus wavelength spectra for a given measurement time) or collapsed (the average, mean, median, or other statistic derived from the time resolved data). In some embodiments, the time, date, and user/patient ID associated with each measurement can also be a part of the information transferred.

In some embodiments, the data from one device can be made available to other devices, for example by transfer to one or more separate computing or storage devices, or storage on a measurement device and accessible by other measurement devices. For convenience of reference, the mechanism that allows data to be transferred among devices is referred to herein as a “centralized server” or a “centralized database”, and encompasses physical computing or storage devices separate from the measurement devices, data storage on one or more measurement devices that can be accessed by other devices, and distributed or shared management of such information among devices. The transfer of data to a centralized server offers several potential benefits including additional security and data analysis and mining of the aggregated data. Furthermore, in some embodiments the measurement device transfers the spectroscopic data to the centralized server and the determination/computation of the hydration or other analyte measurement or measurements is performed on the centralized server. The assay result is then transferred back to one or more noninvasive measurement devices and/or other locations and applications such as cell phones, computers, electronic medical records, tablets, and web applications.

In some embodiments, in addition to or in place of the data discussed above, summary reports and statistics generated locally on a given measurement device can be transferred to a centralized database. Such statistics can include aggregate usage history, error logs, maintenance records, etc. These reports and statistics can be combined with those obtained from one or more devices connected to the centralized database and form a “complete” report or statistics representative of the entire testing program. Such an approach can be advantageous in situations where data transfer rates limit the amount of data that can be transferred or if the volume of data acquired across all devices renders full centralization impractical. It should be noted that the present invention contemplates a range of variants that span the transfer and centralization of one, all, or any combination of the types of data described above.

Centralization Schemes

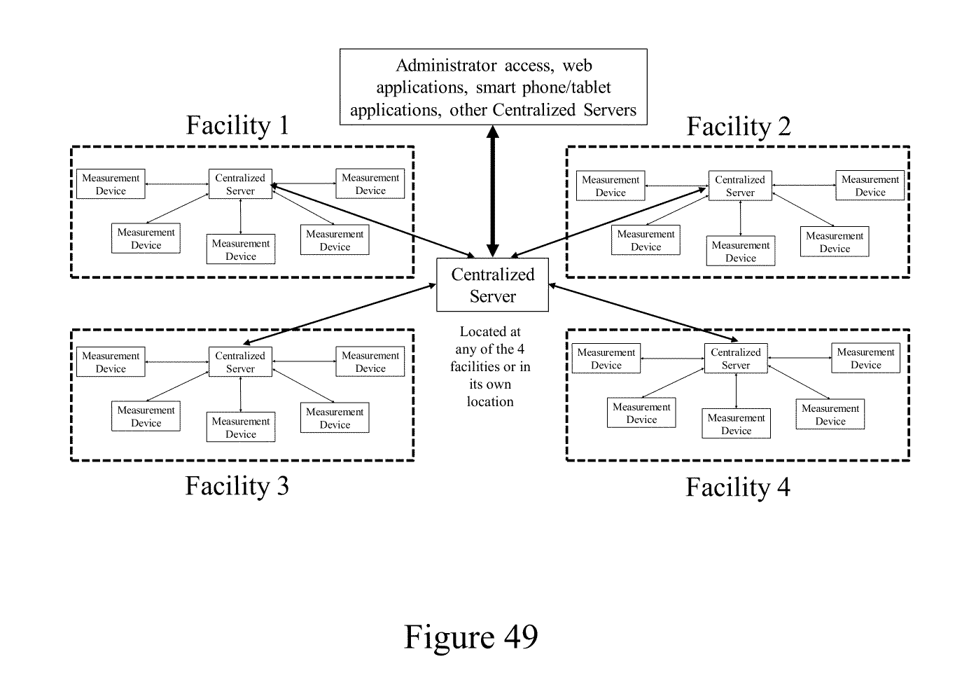

There are multiple embodiments of a centralized database that are suitable for use with the present invention. The following example embodiments are intended to be demonstrative of the range of approaches to centralizing data and are not intended to be limiting. One skilled in the art recognizes that other variants or combinations of the disclosed examples are envisioned by the present invention. In some embodiments, all measurement devices and the centralized database are located at a single site or facility (see FIG. 49). Each device transfers the desired data to the centralized database on the server on demand, at regular intervals, or in real time as data is acquired on a measurement device. The centralized server can also include means for load balancing, prioritization, and scheduling of data transfers to and from measurement devices. In this context, the term “site” can also refer to a facility or group of facilities. Thus, the one or more measurement devices can be collocated in a given part of the site or they can be located in multiple places within the facility such as entry points, triage stations, patient visit rooms, emergency departments, intensive care units, elderly care facilities, etc.

The centralized server/database can be located in a variety of locations such as where one or more of the analyte measurement devices is located, in its own dedicated location, or another secure location within the facility such as a server room. In other embodiments, the centralized server can be one of the analyte measurement devices present on the network or its own dedicated hardware (e.g. a server or servers, computer or computers, located in a secure location). The server can also be located offsite (“remote”) such as a cloud server application and managed by either the customer or a service company (see FIGS. 48 and 49). In some embodiments, multiple centralized databases can be used to ensure data redundancy in case of a security breach or hardware failure (see FIG. 49).

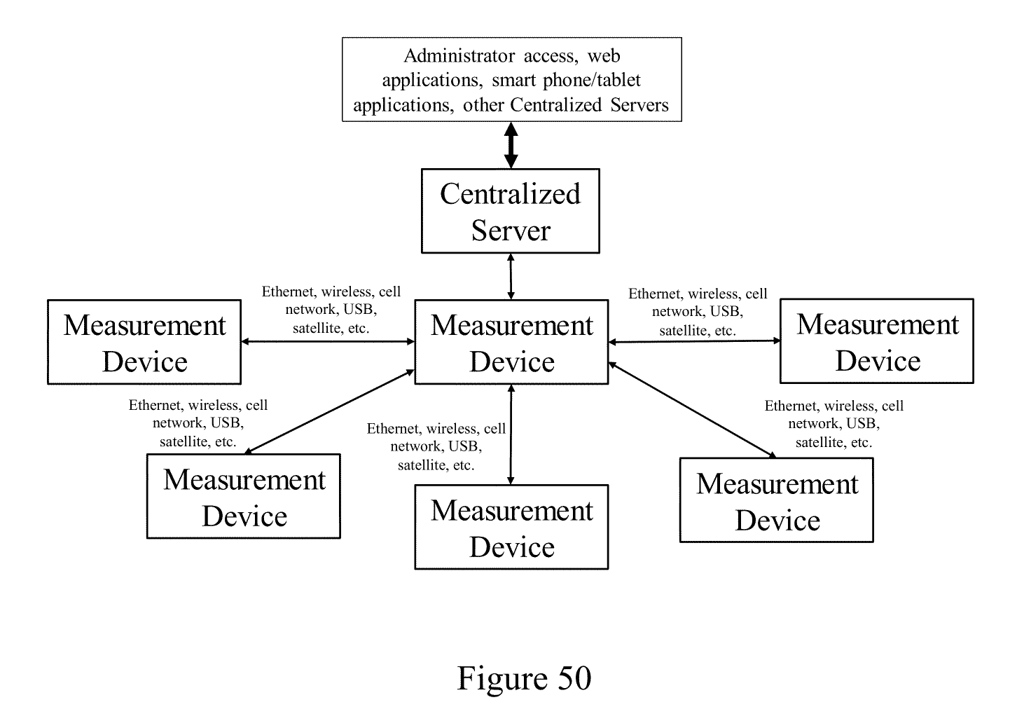

In some embodiments, individually networking all devices to the centralized database (e.g. a hub and spoke approach) might be impractical or expensive (for example in locations where wired Ethernet connection to the centralized database is not available) and equipping all measurement devices with a wireless adapter or similar approach might also be impractical. In such cases, one or more measurement devices can be wired together and a single measurement device configured/equipped to communicate with the centralized server/database (see FIG. 50). In this type of embodiment, one measurement device can effectively serve as a centralized server access point for the other measurement devices. In other embodiments, a customer with multiple facilities can have a centralized database within each facility (see FIG. 49). Those centralized databases can operate independently of each other or communicate and synchronize with each other. In other embodiments, “super” centralized database/servers can integrate the data from the one or more centralized databases/servers (see FIG. 49). These approaches allow customers with multiple sites to share a common centralized server/database and thus integrate all information from their testing program. Alternatively, a remote or “cloud” server can allow a 3^{rd} party to maintain and monitor the centralized data for customers that prefer that type of service.

In some embodiments, the data centralization is “on demand.” For example, a device requests information from the centralized server when it is needed. If the server has the information, it transfers the data to the requesting measurement device. If the server does not have the requested information the server in-turn queries all devices on the network for the desired information and pulls it to the centralized server and then sends the data to the requesting device. In other embodiments, data is pushed to the centralized server immediately upon its acquisition.

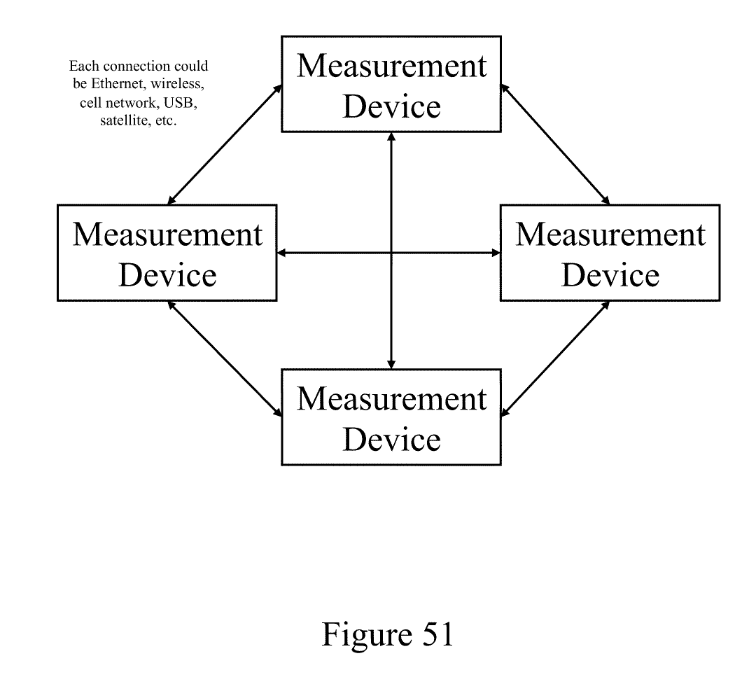

In some embodiments, the centralized server is not implemented as a dedicated piece of hardware, but rather a group of measurement devices. These devices are interconnected (see FIG. 51) in a manner that allows a given device to query information from the other measurement devices on an as needed (e.g. as a person is performing a measurement), scheduled, or real-time basis. Several data storage schemes are viable with such an approach including, but not limited to, each measurement device maintaining a mirrored copy of the same data (e.g. each device transmits its data to the other devices such that they all have full sets of data) or distributed storage where each measurement stores its own data and only transmits information to one or more other measurement devices upon query.

Transfer of Data from a Centralized Database to Devices

An aspect of some embodiments of the present invention is the transfer of data from a centralized server to one or more measurement devices. For example, if a patient switches rooms in a care facility if can be advantageous to transfer past analyte measurement results to the patients new location. In some embodiments this is accomplished by transferring the data from the first device (at the first location) to a centralized database/server which can then transfer that desired information to the device at the new location. Such a process could be initiated at either end by an authorized user, or automatically upon the entry of the patient ID into the device at the new location. Likewise, spectroscopic data and reports can also be transferred in such a manner which allows test administrators and care providers to use measurement devices as access terminals to the contents of the centralized database.

Transfer of Data to and from Additional Software Packages

In some embodiments of the present invention data is transferred from one or more devices, one or more centralized databases, or a combination thereof, to additional software packages within a given facility or customer installation. For example, the measurement results obtained from a patient can be transferred from the measurement device to the patient's electronic medical records with no additional effort or data entry needing to be performed by the medical staff. Alternatively, the measurement results can be transferred to a centralized database which in turn transfers the information to the electronic medical records. One skilled in the art recognizes that other advantageous embodiments of transferring data are readily apparent and that the examples provided are not intended to be limiting. The automated transfer of data to additional software packages offers several advantages including, but not limited to, improved consistency of records, reduced rates of error in records, and reduced labor and overhead costs associated with the hydration test and maintenance or records.

Local and Remote Reporting

While each noninvasive device has internal data reporting capability that can summarize the measurements performed on it, it can be advantageous to combine the databases (results) of multiple devices into a single, centralized database. This can provide an ability to generate unified reports and statistics that combine all tests performed at a given facility or facilities. In some embodiments, the centralized database retains information on which device each measurement came from which can allow the administrator to obtain reports on a single device or group of devices if desired. Furthermore, the administrator can be located in a completely different facility from the measurement devices and/or the centralized database (e.g. any place with internet access) and monitor test results (for example adverse hydration events) in real, or near real, time.

Data Analytics

In some embodiments, application software and/or web applications can be used to allow local and remote access to the centralized database/server and provide advanced data analysis of database contents. The application software can perform advanced analyses such as trend analysis on the data within the centralized database. Such analysis can be significantly more powerful when performed across date acquired from multiple devices (more data generally provides higher confidence in analysis outcomes), which makes a centralized database an attractive approach. Trend analysis can show the therapeutic impact of routine hydration testing over time and across different patient demographics.

In some embodiments of the present invention, application software can be included in order to allow the care givers, hospital staff, testing program administrators, or other authorized parties access to view the contents of the centralized database. These applications can be web based, installed programs, mobile applications such as those used on mobile phones, PDA's, tablets, etc. Application software can also include software installed on one or more measurement devices that enables the administrator or authorized party to access the database at directly at the measurement device.

Methods for Notification of Hydration Measurements Requiring Immediate Action

In some embodiments, a method for notifying the medical staff, care givers, or other entity of a hydration result requiring immediate action is included. There are several approaches to performing this notification including, but not limited to, text messaging, email, mobile applications (e.g. on cell phones, tablets, laptops), programs operating actively or in the background on computers, workstations, or servers, and web-based applications.

Methods for Performing Actions Based on Noninvasive Hydration Results

If a hydration result requires immediate action, the signal indicating that an action is required can be wireless or wired as deemed appropriate for the application. Furthermore, in some embodiments, wireless communication can also be used to report the measurement results or the operational status of the monitoring device to a central monitoring. Furthermore, the action could be taken without involving a person in the process. For example, a hydration assay indicating a patient is dehydrated could result in IV fluid being administered. In this case, the signal from the noninvasive hydration monitor would trigger the opening of a valve or turn on a pump. One skilled in the art will appreciate the wide variety of environments and systems that could can incorporate or function as the analyte monitoring device or in conjunction with the analyte monitoring device of the present invention.

Remote Storage of Enrollment Data/Smart Keys

Additional security, convenience, and policy control may be achieved by storing an individual's data on an external communication device rather than internally in a given measurement device. A “smart key” which can comprise an electronic storage and communication device can store, encrypt, and securely communicate data to the measurement device. For example, in some embodiments the noninvasive measurement data from one or more patients and results can be stored on the smart key. The smart key can then be brought to subsequent devices or data servers. This type of system has several advantages. The system can use existing technology which has been developed for secure encryption, data transmission, and authentication transactions using smart cards/tokens. Further, in applications where an individual might be required to frequently move between multiple locations the use of a smart key allows the stored data to follow the individual.

Some embodiments for remotely storing enrollment data are comprised of a means for storing data, a means for providing power, a means for communication and data transfer. The means for storing data can be any electronic or magnetic storage media consistent with the form factor desired. Some example embodiments of the means for providing power include, but are not limited to, a battery or external RF power via the communication uplink. Some example embodiments of the means for communication and data transfer include, but are not limited to, serial or other secure data protocol via RF, optical, sonic, or direct electrical connections. These components can be combined in many possible form factors. Some examples include car key, plastic transponder, credit card style smart card, employee id badge, lapel pin, etc.

Novel Revenue Models for Noninvasive Hydration Sensors

Another aspect of transferring data to and from a centralized database is that it enables several revenue strategies for the hydration/analyte measurement devices. In some embodiments, the customer can purchase digital measurement “credits” from an appropriate vendor (e.g., the company or entity that placed or sold the test devices to the customer). These credits can then be made available on the centralized database and each measurement performed on a networked device decrements the number of credits available. When the available credits reach zero, additional credits must be purchased or authorized to perform additional measurements. A similar approach is applicable to sites with a single device. In this scenario, the device is effectively also the centralized database and would manage the measurement credits accordingly. Furthermore, in some embodiments the measurement credits have an expiration date that necessitates the purchase of new credits upon expiration.

In installations involving multiple measurement devices, some embodiments of the present invention involve measurement credits that are purchased independently for each measurement device. Other embodiments allow measurement credits to be purchased and managed by one or more centralized servers and/or one or more measurement devices. In such embodiments, for example, high and low usage measurement devices can each draw from the same “centralized” pool of measurement credits.

In some embodiments, the measurement device can be installed based upon a lease program where the lease rate (monthly or otherwise) includes a fixed number of measurement credits. Installations whose usage exceeds the fixed number of measurement credits can purchase additional credits as required. In other embodiments, under-use of the fixed number of measurement credits can result in the un-used credits being “rolled over” into subsequent lease periods.

In some embodiments, the noninvasive measurement device does not need to be networked. In these embodiments, measurement credits can be pre-purchased that allow hydration measurements until the credits are used. The credits can be purchased via a variety of means including obtaining a code from a “gift card” like transaction at a store or an internet purchase on a computer, phone, or other device with internet connectivity. The code is then entered into the device to unlock the corresponding number of measurement credits. Similarly a physical card or key can be can be scanned by the device to enable credits. In some embodiments the code can be ordered and or received by mail.

In some embodiments of the present invention the measurement device tracks the number of measurements performed on it during a given period and transfers that information to a local or remote billing department. The billing department then issues an invoice to the customer. In some embodiments where a customer has multiple devices, the individual device usage numbers can be combined into a single invoice. In some embodiments, the noninvasive device generates and sends the invoice itself and transmits it electronically with instructions for payment. In such cases there is no need to transmit the statistics to the billing department. An example where tracking instrument usage is important is in medical treatment where individual procedures are charged and reimbursed by the patient, insurance companies, Medicaid, Medicare, or a combination thereof. In some embodiments, invoices can be generated on a measurement or patient specific basis in order to facilitate appropriate reimbursement.

Whether a noninvasive measurement device is purchased, leased, or employs the pay per use model as described above the transfer of data to a centralized server allows additional products and services to be offered to the customer. For example, advanced data analysis, trend analysis, and reporting can be implemented on the centralized data. The customer can then pay for access to the analysis results, reports, and remote or local database tools. Such payment can be via subscription fees, one time licenses, or other approaches acceptable to the customer.

Measurement of Intravascular Hydration, Extravascular Hydration, their Combination, And Combination with Other Analytes of Interest

While the total body water (e.g. total hydration state) is of interest in a variety of applications, it is important to note that water is not uniformly distributed in the body at any given time. Depending on a person's specific circumstances (e.g. cause of dehydration or other medical condition or activity), the changes in water concentration in different compartments of the body can vary. For example, blood volume can increase/decrease (hypervolemia/hypovolemia). Extravascular tissues can also increase or decrease in water concentration (hydration, dehydration). However, the two compartments (intravascular and extravascular) do not necessarily exhibit the same behavior under the same conditions. In addition, the blood volume can increase while the extravascular tissues lose water. Each of the possible conditions (both increasing, both decreasing, one up the other down, one down the other up) can have different medical consequences and treatment options. In other words, the difference in the hydration state and behavior of the two compartments is important diagnostic information for many medical conditions as well as the overall hydration state of the body.

In general, the intravascular volume can be used to indicate if a patient is suffering from hypervolemia or hypovolemia. The extravascular compartment can be used to determine the hydration state. Furthermore, knowledge of both the extravascular and intravascular volumes and/or water concentrations can provide not only knowledge of volemic state and the hydration state of the non-blood bearing parts of the body, but it can also provide information regarding how water is being transferred between the intravascular and extravascular compartments.

As a result, clinicians can prefer to have both the intravascular and extravascular hydration state. The NIR light used in the embodiments of the present invention interrogates extravascular tissues as well as the intravascular space. As a result, the spectroscopic signal inherently contains the hydration information for both the intravascular and extravascular compartments. Thus, the present invention satisfies a desire for information that is not currently available in a single test. This information can be used in combination with or in lieu of a noninvasive measurement of total body water. Furthermore, it is recognized that in some applications only a subset of the measurements (intravascular, extravascular, total body hydration) is of interest.

In some embodiments, the intravascular hydration signal is isolated from the extravascular hydration signal through the use of pulse locking. Light penetration depths are sufficient in the NIR wavelengths used in the present invention to interrogate blood compartment (veins, arteries). Since intravascular hydration is related to blood volume, signals that vary with heart rate are indicative of light interacting with blood. Analysis of that part of the spectroscopic signal in turn is indicative of intravascular hydration. The portion of the signal varying with heart rate can be extracted using frequency filtering techniques.

In some embodiments of the present invention, the spectroscopic data acquisition rate exceeds the pulse rate. As a result, the spectroscopic signals acquired over time can be filtered to pass only signals that vary in time consistent with the heart rate. The resulting signal is indicative of the intravascular space and is analyzed for hydration state in order to obtain a measurement of intravascular hydration. The other portion of the signal (not related to the pulse or heart rate) is indicative of the extravascular space and therefore provides a measurement of extravascular hydration.

As the intravascular space is largely comprised of blood, the composition of blood is an important consideration. Blood is made up of plasma (liquid) and blood cells (hematocrit). Commercial optical sensors are used to determine hematocrit levels today. In some embodiments of the present invention, a more accurate determination of intravascular water is obtained by combining a pulse locked measurement method with a hematocrit measurement. For example, for a given person, an increase in hematocrit concentration over time can indicate a decrease in plasma volume. In other words, decreased plasma volume corresponds to dehydration.

In some embodiments of the present invention the noninvasive hydration measurement or measurements (intravascular, extravascular, both) can be combined with additional analyte measurements including, but not limited to, oxygen saturation, hemoglobin, hematocrit, bilirubin, ethanol, glucose, pH, and blood gases. The additional analyte measurements can be obtained from the addition of additional sensors, the noninvasive device that generates the hydration measurement, or a combination thereof. The result is a device that yields a panel of assay results that can help guide health and medical decisions and treatment.

Advantages of Increased Pathlength and Multiple Pathlength Measurements

The propagation of light through tissue depends on both scattering and absorption, both of which vary with wavelength depending on the structural and chemical properties of the tissue being interrogated. Relative to longer wavelengths such as the mid-IR (>2500 nm), short wave NIR contains overtones of water fundamentals that are much weaker in terms of light absorption per unit concentration and path length. As a result, light introduced to the body at these wavelengths can travel significantly further through tissue. Path lengths of several centimeters can be achieved which enables significant penetration through the skin layers and into tissues such as muscles, veins, and arteries. The result is that the light that returns to the skin surface has hydration information of the skin layers, deeper tissues (muscles), and the vascular network within the part of the body interrogated.

Multivariate analysis and knowledge of the optical probe design used by the noninvasive measurement system can be used to extract the hydration signal for each type of tissue measured. The result is the ability to quantify the hydration state in each type of tissue and provide significantly more information than simply the total body water. The difference in hydration between the compartments can be elucidated as well as their relative changes over time. This can be crucial information to medical practitioners, athletes, and persons with medical conditions or medications that can impact the transfer of fluid throughout the body.

Longer path lengths with deeper penetration can be achieved by increasing the separation between illumination and collection fibers in a reflectance optical probe. Reducing the numerical aperture of the optical fibers or other means for introducing and collecting light from the skin. In transmission measurements (illumination and collection are not on the same side of the skin surface), a thicker body part can be used to increase the path length.

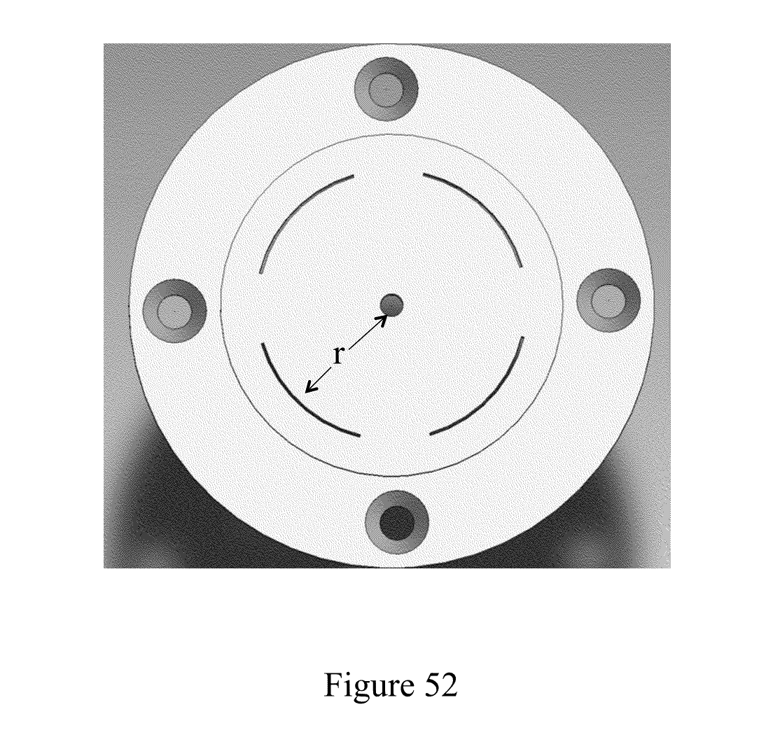

FIG. 52 shows an embodiment of the tissue interface of a noninvasive hydration optical probe that has an increased S-R separation consistent with achieving effective optical path lengths of 1-3 cm through tissue. The central circle is the location where light is collected from the tissue. This circle can be comprised of a single cylinder fabricated from fused silica or other material with acceptable light transmission characteristics. Alternatively, the circle can be filled with a bundle of optical fibers. One skilled in the art recognizes that other mechanisms for collecting the light exiting the tissue within the central circle. The outer ring is the portion of the optical probe surface where light is introduced to the sample. The length and thickness of the outer ring can be varied to increase the total amount of illumination area and therefore alter the throughput of the system. In the embodiment shown in FIG. 52, the outer ring is not continuous. The breaks in the ring allow the entire surface of the sample interface to be fabricated from a single piece of material. The material can be metal, including but not limited to stainless steel or aluminum, plastic, glass, or other suitable material. The primary objectives of the sample interface are to hold the illumination and collection portions of the optical probe in place. The sample interface also preferably allows easy cleaning and user comfort. The radius, r, defines the edge to edge separation between the illumination and collection portions of the optical probe. In some embodiments the radius r is 10 mm, which yields path lengths of light through the tissue of one or more centimeter.

One skilled in the art recognizes that increasing or decreasing the radius, r, will correspondingly increase or decrease the effective path length and depth of penetration through tissue. Likewise, the amount of light decreases with increasing radius. Thus, different embodiments with different values of r achieve a different balance of these effects. For example, if dermal hydration within the skin is the desired measurement, a small r (for example 100 microns) would limit path lengths through tissue to the skin.

In addition to r, the numerical aperture of the illumination and collection optics (be they cylinders, optical fibers, or other suitable optics) can also influence path length and penetration depth. Furthermore, in the embodiment shown in FIG. 52, optical fibers can be arranged in the outer ring such that they launch light into the sample (skin) at an angle. That angle can be inclined towards or away from the center circle. The illumination angle can strongly influence path length and depth of penetration. One skilled in the art recognizes that many alternative geometries of illumination and collection areas can be contemplated, each of which is suitable for the purposes of the present invention.

The concentration of water within the body is not uniform. As a result, light interacting with tissue can encounter differing levels of water in different parts of the tissue. These differences can be important in cases where the concentration in one part of the body is of interest to the exclusion of others. Likewise, some hydration measurements can benefit from the measurement of hydration in more than one part of the body or type of tissue. Monitoring the changes in hydration over time in the various compartments can also be important in some embodiments. As a result, some embodiments of the present invention have optical probes designed to accomplish two objectives: 1) collect light from the tissue that has interacted with one or more types of tissue or compartments (such as intravascular and extravascular), and 2) provide a means for separating the hydration signals of one or more compartments from the signals of other compartments.

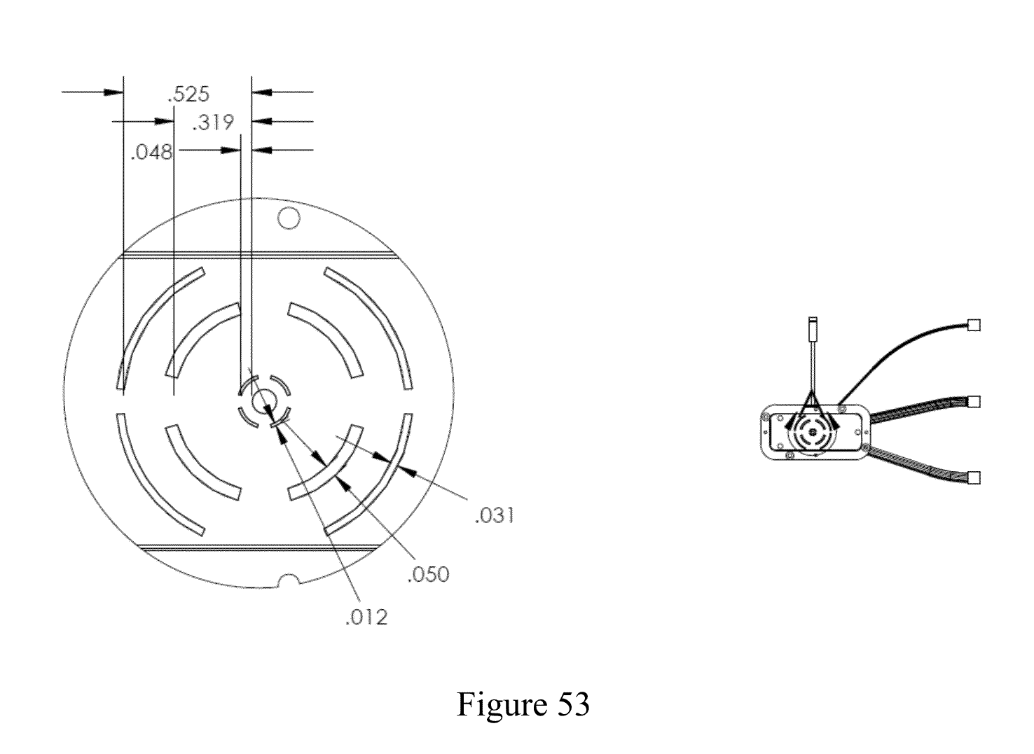

In order to accomplish this, in some embodiments, the optical probe can have multiple channels. Each channel is optimized to interrogate a different depth in the body. FIG. 53 shows a 3 channel optical probe. The first (inner) channel intentionally interrogates the shallow, skin, layers of the body. The next two channels interrogate deeper tissues (muscle, vascular system). As light must pass through the skin to get to the deeper tissues, the first channel can be used to estimate the water signal in the skin and remove it from the signal of the other channels. The net result is improved isolation of the hydration state of the deeper tissues without the influence of the more shallow skin layers.

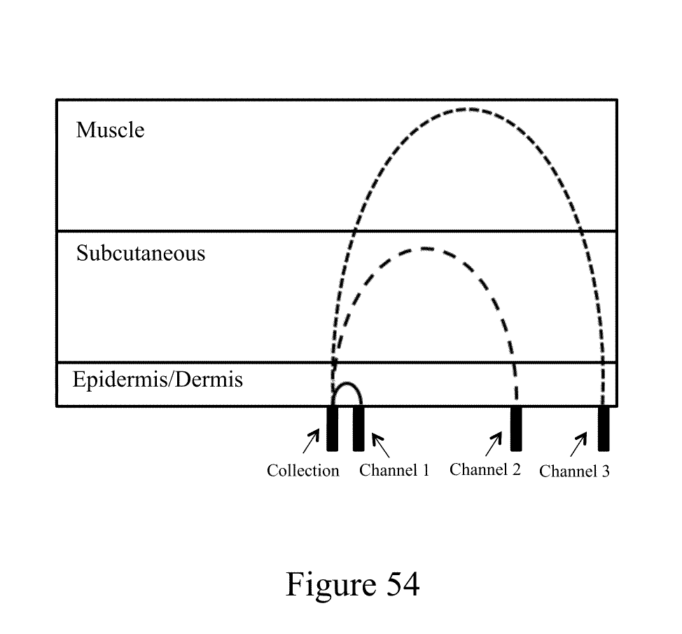

In some embodiments, the signal of each compartment is estimated as well as the corresponding path length through the compartment that gave rise to the signal. The result is that the hydration signal of a given compartment can be normalized to a given path length and then used to remove that signal from the measurements obtained from other channels. FIG. 54 shows a schematic of the utility of multiple-channel optical probes which shows three illumination channels and a single collection channel. Each channel is at a different separation from the collection channel, but are otherwise identical in this embodiment. The average light propagation for a given channel is simplistically represented by an arc. This schematic depiction is for demonstration purposes only as photon propagation through tissue is recognized by one skilled in the art to be significantly more complex than indicated by the arcs.

Each channel can be measured sequentially in order to obtain a spectroscopic signal related to that channel. Alternatively, the channels can be measured according to an encoding scheme such as a Hadamard encoding approach. The spectroscopic signal for each channel is obtained by applying a Hadamard transform to the measured signals obtained from the encoding scheme. One skilled in the art recognizes that there are a variety of encoding and decoding schemes that are applicable to the present invention. In all cases the objective is to obtain a spectroscopic signal for each illumination/collection combination (three in the case of the embodiment shown in FIG. 54).

In the embodiment shown in FIG. 54, the illumination-collection separation for the first channel is designed to constrain optical path lengths and depth of penetration to the epidermis and dermis. It should be noted that, in reality, the dermis and epidermis are distinct layers. However, for simplicity the depiction in FIG. 54 combines the two into a single layer in order to describe the invention. Thus, the spectroscopic signal contains information on the analytes present in the dermis and epidermis and would be analyzed to determine hydration state.

The second channel in FIG. 54 is chosen such that light propagates significantly further through the tissue. However, said light must pass through the 1^{st} layer on the way into the tissue and back through it on the way out of the tissue. Thus, the spectroscopic signal for channel two will be comprised of information from both the dermal/epidermal layer as well as the subcutaneous layer. As a result, in order to isolate the signal or analyte measurement (e.g. hydration state) of the subcutaneous, the contribution of the dermis/epidermis must be removed or compensated. Analysis of the signal differences between channels 1 and 2 allows such removal and compensation. Upon the completion of the compensation, analyte (hydration) measurements are available for both the epidermal/dermal layer as well as the subcutaneous layer.

Similarly, the analyte (hydration) estimates from the first two layers or channels can be used to isolate the signal or analyte measurement from the third tissue layer using the third illumination channel. Consequently, while simplistic, FIG. 54 demonstrates how multiple channels in an optical probe can be used to elucidate analyte (hydration) information in different layers or compartments within the tissue. A similar approach can be used to determine extravascular and intravascular hydration.

While longer path lengths and deeper depths of penetration can be advantageous for interrogating deeper biological tissues, there is a penalty in achievable signal-to-noise ratio due to absorbance and scattering losses. For some embodiments, it is advantageous to selectively adjust the path through tissue by wavelength or wavelength region such that an optimization of path length vs. SNR can be made for each spectral region of interest. In one example embodiment, multiple illumination channels, where each channel is designed to measure a different wavelength region, are arranged such that their physical separation (or numerical aperture, or angle, or combination thereof) from the collection is spaced according to the relative absorbance of their individual spectral regions or individual wavelengths (for laser sources). Alternatively, a single illumination channel can be used in conjunction with multiple collection channels where each collection channel is designed to optimize SNR and path length for a given wavelength region. The illumination channel can introduce light to the sample obtained from any type of suitable light source such as broad-band light sources (blackbody, SLED, etc) or narrower light sources such as LED's or lasers.

One specific embodiment using multiple collection channels would arrange the light collected from the channels such that it is passed through a variable optical transmission filter. The optical transmission filter passes different wavelengths of light at different locations on the filter. An array of photodetectors is placed at the output of the optical filter in order to detect the transmitted light. The collection channels at the sample (tissue) surface are arranged such that the wavelength regions least absorbed by the skin would be furthest away from the illumination channel or channels while those elements corresponding to heavily absorbed wavelength regions would be closest to the illumination channel or channels.

Additional Considerations

Hibernation Mode

Another aspect of the present invention is hibernation mode. In some embodiments, the consumption of electrical power by a measurement device during periods of inactivity can be disadvantageous. In such cases, a device can enter a hibernation mode where power consumption is reduced. The device can enter hibernation mode at predefined times (e.g. overnight), or at some predetermined time after a measurement (e.g. 15 minutes after the completion of a measurement unless another measurement is performed first). The device would “awake” from hibernation upon any user interaction such as contact with the optical touch pad of a device, keypad/keyboard, swipe card reader, touch screen, or proximity detection. An additional advantage of hibernation mode is the increased lifetime of components such as light sources, whose useful life is related to the amount of time they are in use. Thus, hibernation allows those components to be turned off, or operated at a reduced power level, while users are not interacting with the measurement device. The result is that these components can remain in use a longer period of calendar time prior to needing replacement.

In some embodiments, the aggregated data for a ‘team’ could be utilized to ensure group performance/team health; this may prove useful in a military application or severe industrial environment such as desert based mining or oil/gas operations.

Fast Scan and Screening

In some applications of the present invention the throughput requirements (for example, the number of measurements performed per hour) can be accommodated through the use of a “fast scan” or “screening” measurement. In some embodiments, the “fast scan” can be implemented by altering the question posed to the device. For example, the question is often “what is the hydration state of this person?” In a fast scan embodiment, the question could instead be “is this person below a certain level of hydration?” In this approach, the actual concentration isn't important; but rather that it is less than some defined threshold. As a result, less measurement precision can be required and the result can therefore be obtained in a shorter measurement time. For measurements where the person cannot be deemed below the threshold an additional action can be performed. The additional action can take several forms including one or more of the following: a quantitative hydration or other analyte measurement on the same or different measurement device, a repeat “fast scan” on the same or different measurement device, notification of test administrators, measurement of the analyte using a different approach (e.g. blood). In some embodiments, the use of the additional action without a prior “fast scan” measurement is contemplated by the present invention.

Combination with Additional Sensor Techniques

The addition of another technology could possibly enhance the precision of the measurement in the described IR hydration sensor. The use of a microfluidic device such as a fluid channel that creates a path for sweat or other fluid to move across a detector or multiple detectors. The analytes detected could be used to cross reference or corroborate any change noticed from the IR detection system. Another possible technology is the use of an ultrasonic detection device. This device, an ultrasonic emitter and receiver, can be tuned to measure the water content in the deeper tissue.

Those skilled in the art will recognize that the present teachings can be manifested in a variety of forms other than the specific embodiments described and contemplated herein. Accordingly, departures in form and detail can be made without departing from the scope and spirit of the present teachings as described in the appended claims.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. As used herein, the phrase at least one of A, B, and C should be construed to mean a logical (A or B or C), using a non-exclusive logical OR. It should be understood that one or more steps within a method might be executed in different order (or concurrently) without altering the principles of the present disclosure.

In this application, including the definitions below, the term module may be replaced with the term circuit. The term module may refer to, be part of, or include an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor (shared, dedicated, or group) that executes code; memory (shared, dedicated, or group) that stores code executed by a processor; other suitable hardware components that provide the described functionality; or a combination of some or all of the above, such as in a system-on-chip.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, device structures, and technologies are not described in detail.

The terminology used herein is for the purpose of describing particular example embodiments only and is not intended to be limiting. As used herein, the singular forms “a,” “an,” and the may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The term code, as used above, may include software, firmware, and/or microcode, and may refer to programs, routines, functions, classes, and/or objects. The term shared processor encompasses a single processor that executes some or all code from multiple modules. The term group processor encompasses a processor that, in combination with additional processors, executes some or all code from one or more modules. The term shared memory encompasses a single memory that stores some or all code from multiple modules. The term group memory encompasses a memory that, in combination with additional memories, stores some or all code from one or more modules. The term memory may be a subset of the term computer-readable medium. The term computer-readable medium does not encompass transitory electrical and electromagnetic signals propagating through a medium, and may therefore be considered tangible and non-transitory. Non-limiting examples of a non-transitory tangible computer readable medium include nonvolatile memory, volatile memory, magnetic storage, and optical storage.

The apparatuses and methods described in this application may be partially or fully implemented by one or more computer programs executed by one or more processors. The computer programs include processor-executable instructions that are stored on at least one non-transitory tangible computer readable medium. The computer programs may also include and/or rely on stored data.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

We claim:

1. A system to determine a hydration state of a subject, comprising:
(a) a spectroscopic system configured to determine a spectroscopic signal representative of the response of the subject's tissue to incident light;
(b) an analysis system configured to determine intravascular hydration, extravascular hydration, or both, from the spectroscopic signal.

2. A system as in claim 1, wherein the spectroscopic system is configured to determine a spectroscopic signal at a plurality of times spanning at least one heartbeat of the subject, and wherein the analysis system is configured to determine intravascular hydration from one or more components of the spectroscopic signal that varies with the heartbeat of the subject.

3. A system as in claim 2, wherein the spectroscopic system is configured to determine a spectroscopic signal at a plurality of times spanning at least two heartbeats of the subject.

4. A system as in claim 2, wherein the analysis system is configured to determine a frequency corresponding to the heart rate of the subject, and to determine intravascular hydration from components of the spectroscopic signal that vary at the determined frequency.

5. A system as in claim 2, wherein the analysis system is configured to determine extravascular hydration from one or more components of the spectroscopic signal that does not vary with the heartbeat of the subject.

6. A system as in claim 2, wherein the analysis system is configured to determine a frequency corresponding to the heart rate of the subject, and to determine extravascular hydration from the spectroscopic signal after filtering out the determined frequency from the spectroscopic.

7. A system as in claim 1, wherein the analysis system is configured to determine intravascular hydration from the spectroscopic signal according to a first method, and to determine extravascular hydration from the spectroscopic signal according to a second method, wherein the first method is distinct from the second method.

8. A system as in claim 1, wherein the spectroscopic system is configured to determine a plurality of spectroscopic signals representative of the response of the subject's tissue to incident light at a plurality of distinct times, and wherein the analysis system is further configured to determine a time-dependent change in intravascular hydration, extravascular hydration, or both, responsive to the plurality of spectroscopic signals.

9. A system as in claim 1, wherein the analysis system is configured to distinguish hydration from hypervolemia or hypovolemia.

10. A system as in claim 1, wherein the analysis system is further configured to determine an analyte property, other than hydration, from the spectroscopic signal.

11. A system as in claim 1, wherein the analysis system is configured to determine hydration from a model relating spectroscopic signals to hydration.

12. A system as in claim 1, further comprising a hematocrit sensor, and wherein the analysis system is configured hydration from the spectroscopic signal and from the hematocrit sensor output.

13. A system as in claim 1, wherein the incident light is near-infrared light.

14. A method of determining hydration of a subject, comprising:
(a) determining a signal representative of the response of the subject's tissue to incident light at a plurality of wavelengths;
(b) determining intravascular hydration and extravascular hydration from the signal.

15. A method as in claim 14, wherein step (a) comprises determining the response of the subject's tissue to near-infrared light at a plurality of distinct times.

16. A method as in claim 15, wherein the plurality of distinct times span one or more heartbeats of the subject.

17. A method as in claim 16, wherein step (b) comprises determining a frequency corresponding to the heart rate of the subject, and determining intravascular hydration from components of the spectroscopic signal that vary at the determined frequency.

18. A method as in claim 16, wherein step (b) comprises determining a frequency corresponding to the heart rate of the subject, and determining extravascular hydration from components of the spectroscopic signal that do not vary at the determined frequency.

19. A system as in claim 1, wherein the analysis system is configured to determination intravascular hydration from the spectroscopic signal.

20. A system as in claim 1, wherein the analysis system is configured to determination extravascular hydration from the spectroscopic signal.
